(19) United States
(12) Patent Application Publication
(10) Pub. No.: US 2026/0049073 A1
(43) Pub. Date: Feb. 19, 2026

(54) SPECTACLE LENS COMPRISING AN ORGANIC DYE

(71) Applicant: Oakley, Inc., Foothill Ranch, CA (US)

(72) Inventor: Drew HEITZ, Lockbourne, OH (US)

(73) Assignee: Oakley, Inc., Foothill Ranch, CA (US)

(21) Appl. No.: 19/369,606

(22) Filed: Oct. 27, 2025

Related U.S. Application Data
(63) Continuation of application No. PCT/US2024/026164, filed on Apr. 25, 2024.
(60) Provisional application No. 63/462,791, filed on Apr. 28, 2023.

(51) Int. Cl. C07D 413/14; C07D 413/06; C07D 495/04; G02C 7/10

(52) U.S. Cl. CPC C07D 413/14; C07D 413/06; C07D 495/04; G02C 7/10

(57) ABSTRACT

Chroma-enhanced eyewear lenses (eyewear including sunglasses, goggles, visors, etc.) are comprised of absorbing species, including dyes, that selectively attenuate visible light transmission at one or more wavelength bands, and thereby influence the perception of color and impart a chroma enhancement effect. Chroma-enhanced lenses can include optical filters that attenuate light in one or more wavelength bands. The optical filters include the dye or dye mixtures used in chroma-enhanced lenses. The present disclosure provides chroma enhancing aminothiophene-isoxazolone dyes according to Formula I.

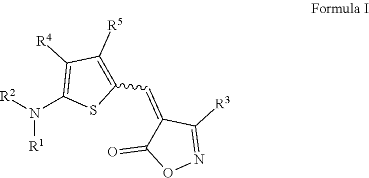

### CROSS REFERENCE TO RELATED APPLICATIONS

This application is a continuation of PCT/US2024/026164 filed Apr. 25, 2024, which claims the benefit of U.S. Provisional Patent Application No. 63/462,791, filed Apr. 28, 2023, which are hereby incorporated by reference in their entireties.

### FIELD

The present disclosure provides organic absorber dyes for chroma-enhanced lenses that selectively reduces light transmission in about the 475 nm to 510 nm region.

### BACKGROUND

Chroma-enhanced eyewear lenses (eyewear including sunglasses, goggles, visors, etc.) are comprised of absorbing species, including dyes, that selectively attenuate visible light transmission at one or more wavelength bands, and thereby influence the perception of color and impart a chroma enhancement effect. Chroma-enhanced lenses can include optical filters that attenuate light in one or more wavelength bands. The optical filters include the dye or dye mixtures used in chroma-enhanced lenses. The optical filter is often optimized for specific applications, including, for example, driving and various sports, e.g., track, golf, and cycling. Dyes that provide the chroma enhancement effect typically absorb selectively in narrow bands, which include the range of about 460 to 510 nm (in the blue-green) and in the range of about 575 to 605 nm (in the yellow-orange). In addition, there can be coatings, polarizing filters, blue cut dyes, deep red absorbing dyes, and broadly absorbing dyes employed in the eyewear to adjust overall transmission, performance, and appearance.

### BRIEF SUMMARY

In one aspect, the present disclosure is directed to a compound of Formula I

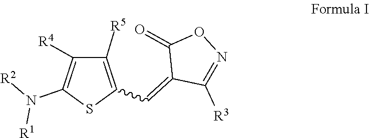

wherein
R¹ and R² are each, independently, C₁-C₆ alkyl, C₁-C₆ alkylene-OH, alkylene C(O)OR⁶, alkylene nitrile, C₁-C₆ haloalkyl, SO₂-haloalkyl, C₁-C₆ alkylene-O-alkyl, alkylene OC(O)NHR⁷, or C₃-C₆ cycloalkyl; or R¹ and R² are taken together with the nitrogen to which they are bound to form an optionally substituted 4- to 7-membered heterocycle;
R³ is C₁-C₆alkyl, C₁-C₆ haloalkyl, C₁-C₆ alkenyl, C₁-C₆ alkynyl, C₁-C₆ alkoxy, SO₂-alkyl, SO₂-haloalkyl, SO₂-aryl, C₁-C₆ cycloalkyl, C₁-C₆ alkylene-O-alkyl, nitrile, C(O)OR⁶, optionally substituted C₆-C₁₀ aryl, or optionally substituted C₃-C₉ heteroaryl;
R⁴ and R⁵ are each, independently, H, D, halo, C₁-C₆ alkyl, C₁-C₆ alkoxy, C₁-C₆ alkenyl, C₁-C₆ alkynyl, C₁-C₆ cycloalkyl, or C₆-C₁₀ aryl; or R⁴ and R⁵ can be taken together with the ring to which they are bound form an optionally substituted C₆-C₁₀ cycloalkyl optionally containing 1-3 heteroatoms;
R⁶ is C₁-C₆ alkyl; and
R⁷ can be H, alkyl, haloalkyl, optionally substituted phenyl, benzyl or phenylalkyl, provided that when R¹ and R² are both ethyl, R³ is not phenyl.

In another aspect, the compound of the disclosure is a compound having Formula I, wherein
R¹ and R² are each, independently, methyl, ethyl, n-propyl, n-butyl, (CH₂)ₘCO₂R⁶, (CH₂)ₘCN, (CH₂)ₘCF₃, or C₃-C₆ cycloalkyl;
or R¹ and R² are taken together with the nitrogen to which they are bound to form a 4- to 7-membered heterocycle, wherein the 4- to 7-membered heterocycle is

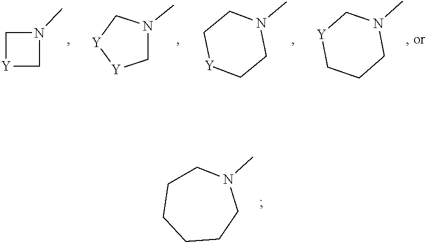

R³ is methyl, ethyl, t-butyl, methoxy, ethoxy, CF₃, CF₂H, CH₂F, CF₂CF₃, CH₂Cl, CCl₃, CF₂Cl, CFCl₂, phenyl, pyridin-4-yl, pyridin-3-yl, pyridine-2-yl, or 4-methoxyphenyl;
R⁴ and R⁵ are each, independently, selected from H, D, methyl, ethyl, i-propyl, t-butyl, n-butyl, halo, methoxy, ethoxy, cycloalkyl, or phenyl;
R⁶ is C₁-C₆ alkyl;
each m is, independently, 1, 2, 3, or 4; and
Y is, independently, CH₂, O, S, SO, SO₂, CF₂, NR⁷, N—C(O)R⁷, N—SO₂—R⁷, N—C(O)OR⁷, N—C(O)N(R⁷)₂, CO, C(H)OH, C(H)OR⁷, C(H)CN, C(H)OC(O)NHR⁷, C(H)CO₂R⁷, C(H)CF₃, C(H)-alkyl, or C(H)-aryl; and
R⁷ can be H, alkyl, haloalkyl, phenyl, benzyl or phenylalkyl.

In another aspect, the compound of the disclosure is a compound having Formula I, wherein
R¹ and R² are each, independently, methyl, ethyl, or n-butyl;
R³ is methyl, ethyl, CF₃, CF₂H, CF₂CF₃, or phenyl; and
R⁴ and R⁵ are each, independently, H, methyl, halo, or phenyl or R⁴ and R⁵ can be taken together with the ring to which they are bound form an optionally substituted C₆-C₁₀ cycloalkyl optionally containing 1-3 heteroatoms.

In another aspect, the compound of the disclosure is a compound having Formula I, wherein
R¹ and R² are taken together with the nitrogen to which they are bound to form the 6-member heterocycle Y or,

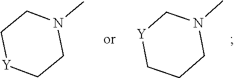

R³ is CF₃ or phenyl;
R⁴ and R⁵ are H; and
Y is C(H)OH or CO.

In another aspect, the compound of the disclosure is a compound having Formula I, wherein
R¹ and R² are taken together with the nitrogen to which they are bound to form the 6-member heterocycle

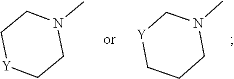

R³ is CF₃ or phenyl;
R⁴ and R⁵ are H;
Y is N—C(O)R⁷, N—C(O)OR⁷ or N—SO₂—R⁷; and
R⁷ is methyl or t-butyl.

In another aspect, the compound of the disclosure is a compound having Formula I, wherein
R¹ and R² are taken together with the nitrogen to which they are bound to form the 6-member heterocycle

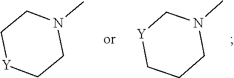

R³ is CF₃ or phenyl;
R⁴ and R⁵ are H; and
Y is O, SO, or SO₂.

In another aspect, the compound of the disclosure is a compound having Formula I, wherein
R¹ and R² are taken together with the nitrogen to which they are attached to form the 5-member or 6-member heterocycle

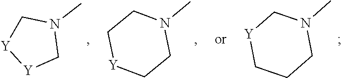

R³ is methyl, CF₃ or phenyl; and
R⁴ and R⁵ are H;
Y is CH₂.

In another aspect, the compound of the disclosure is a compound having Formula I, wherein
R¹ and R² are each, independently, methyl, ethyl, or n-butyl;
or R¹ and R² are taken together with the nitrogen to which they are bound to form the 4- to 7-membered heterocycle

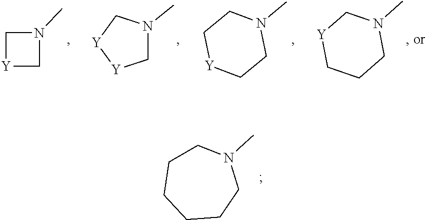

R³ is methyl, CF₃, CF₂H, CF₂CF₃, or phenyl; and
R⁴ and R⁵ are each, independently, H, methyl, bromo, or phenyl;
Y in each instance is, independently, CH₂, O, S, SO, SO₂, C(H)OH, CO, N—C(O)CH₃, N—C(O)O-t-butyl, N—C(O)OCH₃ or N—SO₂—CH₃.

In another aspect, the compound of the disclosure is
4-((5-(dimethylamino)thiophen-2-yl)methylene)-3-(trifluoromethyl)isoxazol-5(4H)-one;
4-((5-(dimethylamino)thiophen-2-yl)methylene)-3-phenylisoxazol-5(4H)-one;
4-((5-(dimethylamino)thiophen-2-yl)methylene)-3-(perfluoroethyl)isoxazol-5(4H)-one;
3-(difluoromethyl)-4-((5-(dimethylamino)thiophen-2-yl)methylene)isoxazol-5(4H)-one;
4-((5-(dimethylamino)thiophen-2-yl)methylene)-3-methylisoxazol-5(4H)-one;
4-((5-(diethylamino)thiophen-2-yl)methylene)-3-(trifluoromethyl)isoxazol-5(4H)-one;
4-((5-(diethylamino)thiophen-2-yl)methylene)-3-phenylisoxazol-5(4H)-one;
4-((5-(dibutylamino)thiophen-2-yl)methylene)-3-phenylisoxazol-5(4H)-one;
4-((5-(dimethylamino)-3-methylthiophen-2-yl)methylene)-3-(trifluoromethyl)isoxazol-5(4H)-one;
4-((5-(dimethylamino)-3-methylthiophen-2-yl)methylene)-3-phenylisoxazol-5(4H)-one;
4-((5-(piperidin-1-yl)thiophen-2-yl)methylene)-3-(trifluoromethyl)isoxazol-5(4H)-one;
3-phenyl-4-((5-(piperidin-1-yl)thiophen-2-yl)methylene)isoxazol-5(4H)-one;
3-(perfluoroethyl)-4-((5-(piperidin-1-yl)thiophen-2-yl)methylene)isoxazol-5(4H)-one;
3-(difluoromethyl)-4-((5-(piperidin-1-yl)thiophen-2-yl)methylene)isoxazol-5(4H)-one;
4-((5-(pyrrolidin-1-yl)thiophen-2-yl)methylene)-3-(trifluoromethyl)isoxazol-5(4H)-one;
3-phenyl-4-((5-(pyrrolidin-1-yl)thiophen-2-yl)methylene)isoxazol-5(4H)-one;
4-((5-morpholinothiophen-2-yl)methylene)-3-(trifluoromethyl)isoxazol-5(4H)-one;
4-((5-morpholinothiophen-2-yl)methylene)-3-phenylisoxazol-5(4H)-one;
tert-butyl-4-(5-((5-oxo-3-(trifluoromethyl)isoxazol-4(5H)-ylidene)methyl)thiophen-2-yl)piperazine-1-carboxylate;
tert-butyl-4-(5-((5-oxo-3-phenylisoxazol-4(5H)-ylidene)methyl)thiophen-2-yl)piperazine-1-carboxylate;
3-phenyl-4-((5-(piperazin-1-yl)thiophen-2-yl)methylene)isoxazol-5(4H)-one;
4-((5-(4-acetylpiperazin-1-yl)thiophen-2-yl)methylene)-3-phenylisoxazol-5(4H)-one;
4-((5-(4-(methylsulfonyl)piperazin-1-yl)thiophen-2-yl)methylene)-3-phenylisoxazol-5(4H)-one;
4-((5-(4-hydroxypiperidin-1-yl)thiophen-2-yl)methylene)-3-(trifluoromethyl)isoxazol-5(4H)-one;
4-((5-(4-hydroxypiperidin-1-yl)thiophen-2-yl)methylene)-3-phenylisoxazol-5(4H)-one;
4-((5-(4-oxopiperidin-1-yl)thiophen-2-yl)methylene)-3-(trifluoromethyl)isoxazol-5(4H)-one;
4-((5-(4-oxopiperidin-1-yl)thiophen-2-yl)methylene)-3-phenylisoxazol-5(4H)-one;
4-((5-thiomorpholinothiophen-2-yl)methylene)-3-(trifluoromethyl)isoxazol-5(4H)-one;
3-phenyl-4-((5-thiomorpholinothiophen-2-yl)methylene)isoxazol-5(4H)-one;
4-((5-(1,1-dioxidothiomorpholino)thiophen-2-yl)methylene)-3-phenylisoxazol-5(4H)-one;
4-((5-(1-oxidothiomorpholino)thiophen-2-yl)methylene)-3-phenylisoxazol-5(4H)-one;
4-((4-bromo-5-(dimethylamino)thiophen-2-yl)methylene)-3-(trifluoromethyl)isoxazol-5(4H)-one;
4-((4-bromo-5-(dimethylamino)thiophen-2-yl)methylene)-3-phenylisoxazol-5(4H)-one;
4-((5-(dimethylamino)-4-phenylthiophen-2-yl)methylene)-3-(trifluoromethyl)isoxazol-5(4H)-one;
3-methyl-4-((5-(piperidin-1-yl)thiophen-2-yl)methylene)isoxazol-5(4H)-one;
4-((5-(1,1-dioxidothiomorpholino)thiophen-2-yl)methylene)-3-(perfluoroethyl)isoxazol-5(4H)-one;
4-((5-(1-oxidothiomorpholino)thiophen-2-yl)methylene)-3-(perfluoroethyl)isoxazol-5(4H)-one;
4-((5-(bis(2-methoxyethyl)amino)thiophen-2-yl)methylene)-3-(trifluoromethyl)isoxazol-5(4H)-one;
4-((5-(azepan-1-yl)thiophen-2-yl)methylene)-3-(trifluoromethyl)isoxazol-5(4H)-one;
4-((5-(azetidin-1-yl)thiophen-2-yl)methylene)-3-(trifluoromethyl)isoxazol-5(4H)-one;
4-((5-(3-hydroxypyrrolidin-1-yl)thiophen-2-yl)methylene)-3-(trifluoromethyl)isoxazol-5(4H)-one;
1-(5-((5-oxo-3-(trifluoromethyl)isoxazol-4(5H)-ylidene)methyl)thiophen-2-yl)pyrrolidin-3-yl phenylcarbamate;
4-((7-(dimethylamino)-2,3-dihydrothieno[3,4-b][1,4]dioxin-5-yl)methylene)-3-(trifluoromethyl)isoxazol-5(4H)-one;
4-((5-(dimethylamino)thiophen-2-yl)methylene)-3-ethylisoxazol-5(4H)-one;
3-(tert-butyl)-4-((5-(dimethylamino)thiophen-2-yl)methylene)isoxazol-5(4H)-one;
3-(tert-butyl)-4-((5-(piperidin-1-yl)thiophen-2-yl)methylene)isoxazol-5(4H)-one;
3-cyclopropyl-4-((5-(dimethylamino)thiophen-2-yl)methylene)isoxazol-5(4H)-one;
4-((5-(dimethylamino)thiophen-2-yl)methylene)-3-(methoxymethyl)isoxazol-5(4H)-one;
3-(butoxymethyl)-4-((5-(dimethylamino)thiophen-2-yl)methylene)isoxazol-5(4H)-one;
methyl 2-(4-((5-(dimethylamino)thiophen-2-yl)methylene)-5-oxo-4,5-dihydroisoxazol-3-yl)acetate;
butyl 2-(4-((5-(dimethylamino)thiophen-2-yl)methylene)-5-oxo-4,5-dihydroisoxazol-3-yl)acetate;
benzyl 2-(4-((5-(dimethylamino)thiophen-2-yl)methylene)-5-oxo-4,5-dihydroisoxazol-3-yl)acetate;
phenethyl 2-(4-((5-(dimethylamino)thiophen-2-yl)methylene)-5-oxo-4,5-dihydroisoxazol-3-yl)acetate;
ethyl 4-((5-(dimethylamino)thiophen-2-yl)methylene)-5-oxo-4,5-dihydroisoxazole-3-carboxylate;
butyl 4-((5-(dimethylamino)thiophen-2-yl)methylene)-5-oxo-4,5-dihydroisoxazole-3-carboxylate;
4-(4-((5-(dimethylamino)thiophen-2-yl)methylene)-5-oxo-4,5-dihydroisoxazol-3-yl)benzonitrile;
4-((5-(dimethylamino)thiophen-2-yl)methylene)-3-(4-(trifluoromethyl)phenyl)isoxazol-5(4H)-one;
4-((5-(dimethylamino)thiophen-2-yl)methylene)-3-(4-methoxyphenyl)isoxazol-5(4H)-one;
4-((5-(dimethylamino)thiophen-2-yl)methylene)-3-(pyridin-4-yl)isoxazol-5(4H)-one; or
4-((4-chloro-5-(dimethylamino)thiophen-2-yl)methylene)-3-(trifluoromethyl)isoxazol-5(4H)-one.

In another aspect, the compound of the disclosure is
(Z)-4-((5-(dimethylamino)thiophen-2-yl)methylene)-3-(trifluoromethyl)isoxazol-5(4H)-one;
(Z)-4-((5-(dimethylamino)thiophen-2-yl)methylene)-3-phenylisoxazol-5(4H)-one;
(Z)-4-((5-(dimethylamino)thiophen-2-yl)methylene)-3-(perfluoroethyl)isoxazol-5(4H)-one;
(Z)-3-(difluoromethyl)-4-((5-(dimethylamino)thiophen-2-yl)methylene)isoxazol-5(4H)-one;
(Z)-4-((5-(dimethylamino)thiophen-2-yl)methylene)-3-methylisoxazol-5(4H)-one;
(Z)-4-((5-(diethylamino)thiophen-2-yl)methylene)-3-(trifluoromethyl)isoxazol-5(4H)-one;
(Z)-4-((5-(diethylamino)thiophen-2-yl)methylene)-3-phenylisoxazol-5(4H)-one;
(Z)-4-((5-(dibutylamino)thiophen-2-yl)methylene)-3-phenylisoxazol-5(4H)-one;
(Z)-4-((5-(dimethylamino)-3-methylthiophen-2-yl)methylene)-3-(trifluoromethyl)isoxazol-5(4H)-one;
(Z)-4-((5-(dimethylamino)-3-methylthiophen-2-yl)methylene)-3-phenylisoxazol-5(4H)-one;
(Z)-4-((5-(piperidin-1-yl)thiophen-2-yl)methylene)-3-(trifluoromethyl)isoxazol-5(4H)-one;
(Z)-3-phenyl-4-((5-(piperidin-1-yl)thiophen-2-yl)methylene)isoxazol-5(4H)-one;
(Z)-3-(perfluoroethyl)-4-((5-(piperidin-1-yl)thiophen-2-yl)methylene)isoxazol-5(4H)-one;
(Z)-3-(difluoromethyl)-4-((5-(piperidin-1-yl)thiophen-2-yl)methylene)isoxazol-5(4H)-one;
(Z)-4-((5-(pyrrolidin-1-yl)thiophen-2-yl)methylene)-3-(trifluoromethyl)isoxazol-5(4H)-one;
(Z)-3-phenyl-4-((5-(pyrrolidin-1-yl)thiophen-2-yl)methylene)isoxazol-5(4H)-one;
(Z)-4-((5-morpholinothiophen-2-yl)methylene)-3-(trifluoromethyl)isoxazol-5(4H)-one;
(Z)-4-((5-morpholinothiophen-2-yl)methylene)-3-phenylisoxazol-5(4H)-one;
tert-butyl (Z)-4-(5-((5-oxo-3-(trifluoromethyl)isoxazol-4(5H)-ylidene)methyl)thiophen-2-yl)piperazine-1-carboxylate;
tert-butyl (Z)-4-(5-((5-oxo-3-phenylisoxazol-4(5H)-ylidene)methyl)thiophen-2-yl)piperazine-1-carboxylate;
(Z)-3-phenyl-4-((5-(piperazin-1-yl)thiophen-2-yl)methylene)isoxazol-5(4H)-one;
(Z)-4-((5-(4-acetylpiperazin-1-yl)thiophen-2-yl)methylene)-3-phenylisoxazol-5(4H)-one;
(Z)-4-((5-(4-(methylsulfonyl)piperazin-1-yl)thiophen-2-yl)methylene)-3-phenylisoxazol-5(4H)-one;
(Z)-4-((5-(4-hydroxypiperidin-1-yl)thiophen-2-yl)methylene)-3-(trifluoromethyl)isoxazol-5(4H)-one;
(Z)-4-((5-(4-hydroxypiperidin-1-yl)thiophen-2-yl)methylene)-3-phenylisoxazol-5(4H)-one;
(Z)-4-((5-(4-oxopiperidin-1-yl)thiophen-2-yl)methylene)-3-(trifluoromethyl)isoxazol-5(4H)-one;
(Z)-4-((5-(4-oxopiperidin-1-yl)thiophen-2-yl)methylene)-3-phenylisoxazol-5(4H)-one;
(Z)-4-((5-thiomorpholinothiophen-2-yl)methylene)-3-(trifluoromethyl)isoxazol-5(4H)-one;
(Z)-3-phenyl-4-((5-thiomorpholinothiophen-2-yl)methylene)isoxazol-5(4H)-one;
(Z)-4-((5-(1,1-dioxidothiomorpholino)thiophen-2-yl)methylene)-3-phenylisoxazol-5(4H)-one;
(Z)-4-((5-(1-oxidothiomorpholino)thiophen-2-yl)methylene)-3-phenylisoxazol-5(4H)-one;
(Z)-4-((4-bromo-5-(dimethylamino)thiophen-2-yl)methylene)-3-(trifluoromethyl)isoxazol-5(4H)-one;
(Z)-4-((4-bromo-5-(dimethylamino)thiophen-2-yl)methylene)-3-phenylisoxazol-5(4H)-one;
(Z)-4-((5-(dimethylamino)-4-phenylthiophen-2-yl)methylene)-3-(trifluoromethyl)isoxazol-5(4H)-one;
(Z)-3-methyl-4-((5-(piperidin-1-yl)thiophen-2-yl)methylene)isoxazol-5(4H)-one;
(Z)-4-((5-(1,1-dioxidothiomorpholino)thiophen-2-yl)methylene)-3-(perfluoroethyl)isoxazol-5(4H)-one;
(Z)-4-((5-(1-oxidothiomorpholino)thiophen-2-yl)methylene)-3-(perfluoroethyl)isoxazol-5(4H)-one;
(Z)-4-((5-(bis(2-methoxyethyl)amino)thiophen-2-yl)methylene)-3-(trifluoromethyl)isoxazol-5(4H)-one;
(Z)-4-((5-(azepan-1-yl)thiophen-2-yl)methylene)-3-(trifluoromethyl)isoxazol-5(4H)-one;
(Z)-4-((5-(azetidin-1-yl)thiophen-2-yl)methylene)-3-(trifluoromethyl)isoxazol-5(4H)-one;
(Z)-4-((5-(3-hydroxypyrrolidin-1-yl)thiophen-2-yl)methylene)-3-(trifluoromethyl)isoxazol-5(4H)-one;
(Z)-1-(5-((5-oxo-3-(trifluoromethyl)isoxazol-4(5H)-ylidene)methyl)thiophen-2-yl)pyrrolidin-3-yl phenylcarbamate;
(Z)-4-((7-(dimethylamino)-2,3-dihydrothieno[3,4-b][1,4]dioxin-5-yl)methylene)-3-(trifluoromethyl)isoxazol-5(4H)-one;
(Z)-4-((5-(dimethylamino)thiophen-2-yl)methylene)-3-ethylisoxazol-5(4H)-one;
(Z)-3-(tert-butyl)-4-((5-(dimethylamino)thiophen-2-yl)methylene)isoxazol-5(4H)-one;
(Z)-3-(tert-butyl)-4-((5-(piperidin-1-yl)thiophen-2-yl)methylene)isoxazol-5(4H)-one;
(Z)-3-cyclopropyl-4-((5-(dimethylamino)thiophen-2-yl)methylene)isoxazol-5(4H)-one;
(Z)-4-((5-(dimethylamino)thiophen-2-yl)methylene)-3-(methoxymethyl)isoxazol-5(4H)-one;
(Z)-3-(butoxymethyl)-4-((5-(dimethylamino)thiophen-2-yl)methylene)isoxazol-5(4H)-one;
methyl (Z)-2-(4-((5-(dimethylamino)thiophen-2-yl)methylene)-5-oxo-4,5-dihydroisoxazol-3-yl)acetate;
butyl (Z)-2-(4-((5-(dimethylamino)thiophen-2-yl)methylene)-5-oxo-4,5-dihydroisoxazol-3-yl)acetate;
benzyl (Z)-2-(4-((5-(dimethylamino)thiophen-2-yl)methylene)-5-oxo-4,5-dihydroisoxazol-3-yl)acetate;
phenethyl (Z)-2-(4-((5-(dimethylamino)thiophen-2-yl)methylene)-5-oxo-4,5-dihydroisoxazol-3-yl)acetate;
ethyl (Z)-4-((5-(dimethylamino)thiophen-2-yl)methylene)-5-oxo-4,5-dihydroisoxazole-3-carboxylate;
butyl (Z)-4-((5-(dimethylamino)thiophen-2-yl)methylene)-5-oxo-4,5-dihydroisoxazole-3-carboxylate;
(Z)-4-(4-((5-(dimethylamino)thiophen-2-yl)methylene)-5-oxo-4,5-dihydroisoxazol-3-yl)benzonitrile;
(Z)-4-((5-(dimethylamino)thiophen-2-yl)methylene)-3-(4-(trifluoromethyl)phenyl)isoxazol-5(4H)-one;
(Z)-4-((5-(dimethylamino)thiophen-2-yl)methylene)-3-(4-methoxyphenyl)isoxazol-5(4H)-one;
(Z)-4-((5-(dimethylamino)thiophen-2-yl)methylene)-3-(pyridin-4-yl)isoxazol-5(4H)-one; or
(Z)-4-((4-chloro-5-(dimethylamino)thiophen-2-yl)methylene)-3-(trifluoromethyl)isoxazol-5(4H)-one.

In another aspect, the compound of the disclosure is
(E)-4-((5-(dimethylamino)thiophen-2-yl)methylene)-3-(trifluoromethyl)isoxazol-5(4H)-one;
(E)-4-((5-(dimethylamino)thiophen-2-yl)methylene)-3-phenylisoxazol-5(4H)-one;
(E)-4-((5-(dimethylamino)thiophen-2-yl)methylene)-3-(perfluoroethyl)isoxazol-5(4H)-one;
(E)-3-(difluoromethyl)-4-((5-(dimethylamino)thiophen-2-yl)methylene)isoxazol-5(4H)-one;
(E)-4-((5-(dimethylamino)thiophen-2-yl)methylene)-3-methylisoxazol-5(4H)-one;
(E)-4-((5-(diethylamino)thiophen-2-yl)methylene)-3-(trifluoromethyl)isoxazol-5(4H)-one;
(E)-4-((5-(diethylamino)thiophen-2-yl)methylene)-3-phenylisoxazol-5(4H)-one;
(E)-4-((5-(dibutylamino)thiophen-2-yl)methylene)-3-phenylisoxazol-5(4H)-one;
(E)-4-((5-(dimethylamino)-3-methylthiophen-2-yl)methylene)-3-(trifluoromethyl)isoxazol-5(4H)-one;
(E)-4-((5-(dimethylamino)-3-methylthiophen-2-yl)methylene)-3-phenylisoxazol-5(4H)-one;
(E)-4-((5-(piperidin-1-yl)thiophen-2-yl)methylene)-3-(trifluoromethyl)isoxazol-5(4H)-one;
(E)-3-phenyl-4-((5-(piperidin-1-yl)thiophen-2-yl)methylene)isoxazol-5(4H)-one;
(E)-3-(perfluoroethyl)-4-((5-(piperidin-1-yl)thiophen-2-yl)methylene)isoxazol-5(4H)-one;
(E)-3-(difluoromethyl)-4-((5-(piperidin-1-yl)thiophen-2-yl)methylene)isoxazol-5(4H)-one;
(E)-4-((5-(pyrrolidin-1-yl)thiophen-2-yl)methylene)-3-(trifluoromethyl)isoxazol-5(4H)-one;
(E)-3-phenyl-4-((5-(pyrrolidin-1-yl)thiophen-2-yl)methylene)isoxazol-5(4H)-one;
(E)-4-((5-morpholinothiophen-2-yl)methylene)-3-(trifluoromethyl)isoxazol-5(4H)-one;
(E)-4-((5-morpholinothiophen-2-yl)methylene)-3-phenylisoxazol-5(4H)-one;
tert-butyl (E)-4-(5-((5-oxo-3-(trifluoromethyl)isoxazol-4(5H)-ylidene)methyl)thiophen-2-yl)piperazine-1-carboxylate;
tert-butyl (E)-4-(5-((5-oxo-3-phenylisoxazol-4(5H)-ylidene)methyl)thiophen-2-yl)piperazine-1-carboxylate;
(E)-3-phenyl-4-((5-(piperazin-1-yl)thiophen-2-yl)methylene)isoxazol-5(4H)-one;
(E)-4-((5-(4-acetylpiperazin-1-yl)thiophen-2-yl)methylene)-3-phenylisoxazol-5(4H)-one;
(E)-4-((5-(4-(methylsulfonyl)piperazin-1-yl)thiophen-2-yl)methylene)-3-phenylisoxazol-5(4H)-one;
(E)-4-((5-(4-hydroxypiperidin-1-yl)thiophen-2-yl)methylene)-3-(trifluoromethyl)isoxazol-5(4H)-one;
(E)-4-((5-(4-hydroxypiperidin-1-yl)thiophen-2-yl)methylene)-3-phenylisoxazol-5(4H)-one;
(E)-4-((5-(4-oxopiperidin-1-yl)thiophen-2-yl)methylene)-3-(trifluoromethyl)isoxazol-5(4H)-one;
(E)-4-((5-(4-oxopiperidin-1-yl)thiophen-2-yl)methylene)-3-phenylisoxazol-5(4H)-one;
(E)-4-((5-thiomorpholinothiophen-2-yl)methylene)-3-(trifluoromethyl)isoxazol-5(4H)-one;
(E)-3-phenyl-4-((5-thiomorpholinothiophen-2-yl)methylene)isoxazol-5(4H)-one;
(E)-4-((5-(1,1-dioxidothiomorpholino)thiophen-2-yl)methylene)-3-phenylisoxazol-5(4H)-one;
(E)-4-((5-(1-oxidothiomorpholino)thiophen-2-yl)methylene)-3-phenylisoxazol-5(4H)-one;
(E)-4-((4-bromo-5-(dimethylamino)thiophen-2-yl)methylene)-3-(trifluoromethyl)isoxazol-5(4H)-one;
(E)-4-((4-bromo-5-(dimethylamino)thiophen-2-yl)methylene)-3-phenylisoxazol-5(4H)-one;
(E)-4-((5-(dimethylamino)-4-phenylthiophen-2-yl)methylene)-3-(trifluoromethyl)isoxazol-5(4H)-one;
(E)-3-methyl-4-((5-(piperidin-1-yl)thiophen-2-yl)methylene)isoxazol-5(4H)-one;
(E)-4-((5-(1,1-dioxidothiomorpholino)thiophen-2-yl)methylene)-3-(perfluoroethyl)isoxazol-5(4H)-one;
(E)-4-((5-(1-oxidothiomorpholino)thiophen-2-yl)methylene)-3-(perfluoroethyl)isoxazol-5(4H)-one;
(E)-4-((5-(bis(2-methoxyethyl)amino)thiophen-2-yl)methylene)-3-(trifluoromethyl)isoxazol-5(4H)-one;
(E)-4-((5-(azepan-1-yl)thiophen-2-yl)methylene)-3-(trifluoromethyl)isoxazol-5(4H)-one;
(E)-4-((5-(azetidin-1-yl)thiophen-2-yl)methylene)-3-(trifluoromethyl)isoxazol-5(4H)-one;
(E)-4-((5-(3-hydroxypyrrolidin-1-yl)thiophen-2-yl)methylene)-3-(trifluoromethyl)isoxazol-5(4H)-one;
(E)-1-(5-((5-oxo-3-(trifluoromethyl)isoxazol-4(5H)-ylidene)methyl)thiophen-2-yl)pyrrolidin-3-yl phenylcarbamate;
(E)-4-((7-(dimethylamino)-2,3-dihydrothieno[3,4-b][1,4]dioxin-5-yl)methylene)-3-(trifluoromethyl)isoxazol-5(4H)-one;
(E)-4-((5-(dimethylamino)thiophen-2-yl)methylene)-3-ethylisoxazol-5(4H)-one;
(E)-3-(tert-butyl)-4-((5-(dimethylamino)thiophen-2-yl)methylene)isoxazol-5(4H)-one;
(E)-3-(tert-butyl)-4-((5-(piperidin-1-yl)thiophen-2-yl)methylene)isoxazol-5(4H)-one;
(E)-3-cyclopropyl-4-((5-(dimethylamino)thiophen-2-yl)methylene)isoxazol-5(4H)-one;
(E)-4-((5-(dimethylamino)thiophen-2-yl)methylene)-3-(methoxymethyl)isoxazol-5(4H)-one;
(E)-3-(butoxymethyl)-4-((5-(dimethylamino)thiophen-2-yl)methylene)isoxazol-5(4H)-one;
methyl (E)-2-(4-((5-(dimethylamino)thiophen-2-yl)methylene)-5-oxo-4,5-dihydroisoxazol-3-yl)acetate;
butyl (E)-2-(4-((5-(dimethylamino)thiophen-2-yl)methylene)-5-oxo-4,5-dihydroisoxazol-3-yl)acetate;
benzyl (E)-2-(4-((5-(dimethylamino)thiophen-2-yl)methylene)-5-oxo-4,5-dihydroisoxazol-3-yl)acetate;
phenethyl (E)-2-(4-((5-(dimethylamino)thiophen-2-yl)methylene)-5-oxo-4,5-dihydroisoxazol-3-yl)acetate;
ethyl (E)-4-((5-(dimethylamino)thiophen-2-yl)methylene)-5-oxo-4,5-dihydroisoxazole-3-carboxylate;
butyl (E)-4-((5-(dimethylamino)thiophen-2-yl)methylene)-5-oxo-4,5-dihydroisoxazole-3-carboxylate;
(E)-4-(4-((5-(dimethylamino)thiophen-2-yl)methylene)-5-oxo-4,5-dihydroisoxazol-3-yl)benzonitrile;
(E)-4-((5-(dimethylamino)thiophen-2-yl)methylene)-3-(4-(trifluoromethyl)phenyl)isoxazol-5(4H)-one;
(E)-4-((5-(dimethylamino)thiophen-2-yl)methylene)-3-(4-methoxyphenyl)isoxazol-5(4H)-one;
(E)-4-((5-(dimethylamino)thiophen-2-yl)methylene)-3-(pyridin-4-yl)isoxazol-5(4H)-one; or
(E)-4-((4-chloro-5-(dimethylamino)thiophen-2-yl)methylene)-3-(trifluoromethyl)isoxazol-5(4H)-one.

In one aspect, the present disclosure is directed to an optical filter comprising one or more compounds of Formula I, wherein the optical filter selectively blocks visible light within a spectral band of wavelengths ranging from about 475 nm to about 510 nm.

In another aspect, the optical filter, further comprises at least one chroma-enhancing compound that is not a compound of Formula I.

In another aspect, the optical filter comprises a polymeric material.

In some aspects, the one or more compounds of Formula I are selected from the group consisting of
4-((5-(dimethylamino)thiophen-2-yl)methylene)-3-phenylisoxazol-5(4H)-one;
4-((5-(dimethylamino)thiophen-2-yl)methylene)-3-(perfluoroethyl)isoxazol-5(4H)-one;
3-(difluoromethyl)-4-((5-(dimethylamino)thiophen-2-yl)methylene)isoxazol-5(4H)-one;
4-((5-(dimethylamino)thiophen-2-yl)methylene)-3-methylisoxazol-5(4H)-one;
4-((5-(dimethylamino)-3-methylthiophen-2-yl)methylene)-3-(trifluoromethyl)isoxazol-5(4H)-one;
4-((5-(dimethylamino)thiophen-2-yl)methylene)-3-(trifluoromethyl)isoxazol-5(4H)-one;
4-((5-(dimethylamino)-3-methylthiophen-2-yl)methylene)-3-phenylisoxazol-5(4H)-one;
4-((5-(piperidin-1-yl)thiophen-2-yl)methylene)-3-(trifluoromethyl)isoxazol-5(4H)-one;
3-phenyl-4-((5-(piperidin-1-yl)thiophen-2-yl)methylene)isoxazol-5(4H)-one;
3-(perfluoroethyl)-4-((5-(piperidin-1-yl)thiophen-2-yl)methylene)isoxazol-5(4H)-one;
3-(difluoromethyl)-4-((5-(piperidin-1-yl)thiophen-2-yl)methylene)isoxazol-5(4H)-one;
4-((5-(pyrrolidin-1-yl)thiophen-2-yl)methylene)-3-(trifluoromethyl)isoxazol-5(4H)-one;
3-phenyl-4-((5-(pyrrolidin-1-yl)thiophen-2-yl)methylene)isoxazol-5(4H)-one;
4-((5-morpholinothiophen-2-yl)methylene)-3-(trifluoromethyl)isoxazol-5(4H)-one;
4-((5-morpholinothiophen-2-yl)methylene)-3-phenylisoxazol-5(4H)-one;
3-phenyl-4-((5-(piperazin-1-yl)thiophen-2-yl)methylene)isoxazol-5(4H)-one;
4-((5-(4-(methylsulfonyl)piperazin-1-yl)thiophen-2-yl)methylene)-3-phenylisoxazol-5(4H)-one;
4-((5-thiomorpholinothiophen-2-yl)methylene)-3-(trifluoromethyl)isoxazol-5(4H)-one;
3-phenyl-4-((5-thiomorpholinothiophen-2-yl)methylene)isoxazol-5(4H)-one;
4-((5-(1,1-dioxidothiomorpholino)thiophen-2-yl)methylene)-3-phenylisoxazol-5(4H)-one;
4-((5-(1-oxidothiomorpholino)thiophen-2-yl)methylene)-3-phenylisoxazol-5(4H)-one;
3-methyl-4-((5-(piperidin-1-yl)thiophen-2-yl)methylene)isoxazol-5(4H)-one;
4-((5-(1,1-dioxidothiomorpholino)thiophen-2-yl)methylene)-3-(perfluoroethyl)isoxazol-5(4H)-one; and
4-((5-(1-oxidothiomorpholino)thiophen-2-yl)methylene)-3-(perfluoroethyl)isoxazol-5(4H)-one.

In another aspect, the present disclosure is directed to the optical filter, wherein the compound is (E)-4-((5-(diethylamino)thiophen-2-yl)methylene)-3-(trifluoromethyl)isoxazol-5(4H)-one, (Z)-4-((5-(diethylamino)thiophen-2-yl)methylene)-3-(trifluoromethyl)isoxazol-5(4H)-one, or a combination thereof.

In another aspect, the present disclosure is directed to the optical filter, wherein the compound is (E)-4-((5-(dimethylamino)thiophen-2-yl)methylene)-3-methylisoxazol-5(4H)-one, (Z)-4-((5-(dimethylamino)thiophen-2-yl)methylene)-3-methylisoxazol-5(4H)-one, or a combination thereof.

In another aspect, the present disclosure is directed the optical filter, wherein the compound is (E)-4-((5-morpholinothiophen-2-yl)methylene)-3-(trifluoromethyl)isoxazol-5(4H)-one, (Z)-4-((5-morpholinothiophen-2-yl)methylene)-3-(trifluoromethyl)isoxazol-5(4H)-one, or a combination thereof.

In another aspect, the present disclosure is directed the optical filter, wherein the compound is (E)-4-((5-(dimethylamino)thiophen-2-yl)methylene)-3-(trifluoromethyl)isoxazol-5(4H)-one, (Z)-4-((5-(dimethylamino)thiophen-2-yl)methylene)-3-(trifluoromethyl)isoxazol-5(4H)-one, or a combination thereof.

In another aspect, the present disclosure is directed to the optical filter, wherein the optical filter has a peak optical density of between about 0.05 and about 6.00 OD.

In another aspect, the present disclosure is directed to the optical filter, wherein the one or more compounds is present in the optical filter in a range of about 0.0002% weight to about 6% weight.

In one aspect, the present disclosure is directed to a lens for eyewear comprising a lens body and an optical filter, wherein the optical filter comprises one or more compounds having Formula I.

In another aspect, the present disclosure is directed to the lens for eyewear, wherein optical filter selectively blocks visible light within a spectral band of wavelengths ranging from about 475 nm to about 510 nm.

In another aspect, the present disclosure is directed to the lens for eyewear, wherein the optical filter further comprises at least one chroma-enhancing compound that is not a compound of Formula I.

In another aspect, the present disclosure is directed to the lens for eyewear, wherein the optical filter is the lens body or at least partially incorporated into the lens body.

In another aspect, the present disclosure is directed to the lens for eyewear, wherein the optical filter is at least partially incorporated into a lens coating, an adhering layer, a polarizing layer, a photochromic layer, an anti-reflection layer, or is at least partially incorporated into a combination of coatings and layers.

In another aspect, the present disclosure is directed to the lens for eyewear, wherein the optical filter has a peak optical density of between about 0.05 and about 6.00 OD.

In another aspect, the present disclosure is directed to the lens for eyewear, wherein the at least one or more compounds present in the lens in a range of about 0.0002% weight to about 6% weight.

In one aspect, the present disclosure is directed to a eyewear comprising a lens wherein the lens comprises a lens body; and an optical filter, wherein the optical filter comprises one or more compounds having Formula I.

In another aspect, the present disclosure is directed to the eyewear, wherein the optical filter selectively blocks visible light within a spectral band of wavelengths ranging from about 475 nm to about 510 nm.

In another aspect, the present disclosure is directed to the eyewear, wherein the optical filter further comprises at least one chroma-enhancing compound that is not a compound of Formula I.

In another aspect, the present disclosure is directed to the eyewear, wherein the optical filter is the lens body or at least partially incorporated into the lens body.

In another aspect, the present disclosure is directed to the eyewear, wherein the optical filter is at least partially incorporated into a lens coating, an adhering layer, a polarizing layer, a photochromic layer, an anti-reflection layer, is at least partially incorporated into or a combination of coatings and layers.

In another aspect, the present disclosure is directed to the eyewear, wherein the the optical filter has a peak optical density of between about 0.05 and about 6.00 OD.

In another aspect, the present disclosure is directed to the eyewear, wherein the at least one or more compounds is present in the lens in a range of about 0.0002% weight to about 6% weight.

Additional aspects and advantages of the disclosure will be set forth, in part, in the description that follows, and will flow from the description, or can be learned by practice of the disclosure.

It is to be understood that both the foregoing summary and the following detailed description are exemplary and explanatory only, and do not restrict the scope of the claims.

### BRIEF DESCRIPTION OF THE DRAWINGS

FIG. 1 illustrates a perspective view of an eyewear configured to provide a chroma enhancement, according to some aspects.

FIG. 1A illustrates an aspect of a lens that can be included in the eyewear depicted in FIG. 1.

FIG. 1B illustrates another aspect of a lens that can be included in the eyewear depicted in FIG. 1, the lens comprising an optical filter including a variable filter component and a static filter component.

FIGS. 2A and 2B depict optical characteristics of an optical filter formulated into a lens using about 11 mg of Compound 1 per pound of resin, configured to provide chroma enhancement, for an eyewear, according to some aspects.

FIG. 3 depicts optical characteristics of an optical filter formulated into a lens using about 14 mg of Compound 2 per pound of resin, configured to provide chroma enhancement, for an eyewear, according to some aspects.

FIG. 4 depicts optical characteristics of an optical filter formulated into a lens using about 12 mg of Compound 6 per pound of resin, configured to provide chroma enhancement, for an eyewear, according to some aspects.

### DETAILED DESCRIPTION

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

The present disclosure generally relates to chroma-enhancing dyes, methods of making the chroma-enhancing dyes, method of using these dyes, and various lenses and other components as detailed herein comprising these dyes.

### Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular aspects, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification will control.

The articles “a,” “an,” and “the” are used herein to refer to one or to more than one (i.e., to at least one) of the grammatical object of the article. By way of example, “an element” means one element or more than one element. As used herein, the use of “or” means “and/or” unless expressly stated or understood by one skilled in the art. In the context of a multiple dependent claim, the use of “or” refers back to more than one preceding independent or dependent claim.

As used herein, the term “about” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 10% (e.g., up to 5%, or up to 1%) of a given value.

The term “alkyl,” used either alone or in compound words such as “haloalkyl” includes straight-chain and branched alkyl, example of which include C₁-C₁₂ alkyls and C₁-C₆ alkyls, such as, methyl, ethyl, n-propyl, i-propyl, and the different butyl, pentyl and hexyl isomers. In some aspects the alkyl group can be optionally substituted.

The term “alkenyl” includes straight-chain and branched alkenes such as ethenyl, 1-propenyl, 2-propenyl, and the different butenyl, pentenyl and hexenyl isomers. In some aspects the alkenyl group can be optionally substituted.

The term “alkenyl” also includes polyenes such as 1,2-propadienyl and 2,4-hexadienyl. “Alkynyl” includes straight-chain and branched alkynes such as ethynyl, 1-propynyl, 2-propynyl, and the different butynyl, pentynyl and hexynyl isomers.

The term “alkynyl” can also include moieties comprised of multiple triple bonds such as 2,5-hexadiynyl and the like. In some aspects the alkynyl group can be optionally substituted.

The term “alkylene” denotes a straight-chain or branched alkanediyl. Examples of “alkylene” include CH₂, CH₂CH₂, CH(CH₃), CH₂CH₂CH₂, CH₂CH(CH₃), and the different butylene isomers. In some aspects the alkylene group can be optionally substituted.

The term “aryl” denotes an aromatic ring system having six to ten carbon atoms, i.e., C₆-C₁₀ aryl. Non-limiting exemplary aryl groups include phenyl, naphthyl, indenyl, and azulenyl groups. In some aspects the aryl group can be optionally substituted.

The term “cycloalkyl” denotes a C₃₋₁₂ cycloalkyl or, in certain aspects, a C₃₋₆ cycloalkyl, for example, cyclopropyl, cyclobutyl, cyclopentyl and cyclohexyl.

The term “heterocycle” denotes a ring wherein at least one of the atoms forming the ring backbone is other than carbon. Unless otherwise indicated, a heterocyclic ring can be a saturated, partially unsaturated, or fully unsaturated ring. When a fully unsaturated heterocyclic ring satisfies Hückel's rule, then said ring is also called a “heteroaryl” or aromatic heterocyclic ring. “Saturated heterocyclic ring” refers to a heterocyclic ring containing only single bonds between ring members. In some aspects the heterocycle or heteroaryl group can be optionally substituted.

The term “nitrile” as used herein by itself or as part of another group refers to the group —CN.

The term “halo” either alone or in compound words such as “haloalkyl,” or when used in descriptions such as “alkyl substituted with halo” includes fluorine, chlorine, bromine or iodine (—Cl, —F, —Br, or —I). Further, when used in compound words such as “haloalkyl,” or when used in descriptions such as “alkyl substituted with halo” said alkyl can be partially or fully substituted with halo atoms which can be the same or different. Examples of “haloalkyl” or “alkyl substituted with halo” include CF₃, CHF₂, ClCH₂, CF₂Cl, CFCl₂, CCl₃, CF₃CH₂, and CF₃CCl₂.

The term “alkoxy” a group having the formula —OR′ wherein R′ is, in some aspects, C₁₋₆ alkyl, for example, methoxy, ethoxy, n-propyloxy, i-propyloxy, and the different butoxy, pentoxy and hexyloxy isomers. “Alkenyloxy” includes straight-chain and branched alkenyl attached to and linked through an oxygen atom. Examples of “alkenyloxy” include H₂C≡CHCH₂O and CH₃CH═CHCH₂O. “Alkynyloxy” includes straight-chain and branched alkynyloxy moieties. Examples of “alkynyloxy” include HCECCH₂O and CH₃CECCH₂O.

The phrase “optionally substituted” means that the number of substituents can be zero. Unless otherwise indicated, optionally substituted groups can be substituted with as many optional substituents as can be accommodated by replacing a hydrogen atom with a non-hydrogen substituent on any available carbon or nitrogen atom. Commonly, the number of optional substituents (when present) ranges from 1 to 3.

The term “phenyl” as used herein refers to the radical C₆H₅, derived from benzene by removal of a hydrogen atom.

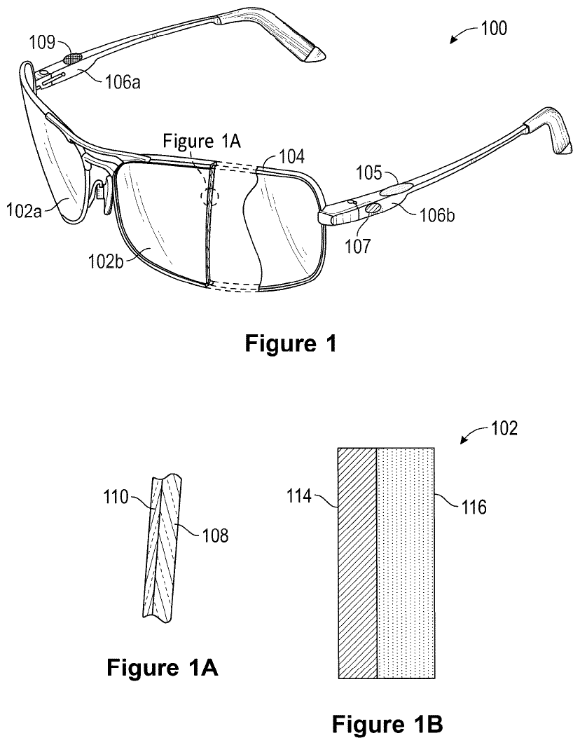

The term “lens” as used herein refers to a piece of transparent material (such as glass, polycarbonate, polyurethane, resin, etc.) that has two opposite regular surfaces either both curved or one curved and the other planar and that is used either singly or combined to form an image by focusing rays of light. The term can refer to lenses used in eyewear including but not limited to general-purpose eyewear, special-purpose eyewear, sunglasses, eyeshields, eyewear incorporated into headworn support (such as visors for helmets), visors, driving glasses, sporting glasses, goggles, vision-correcting eyewear, prescription and non-prescription eyeglasses, color vision deficiency eyewear, indoor eyewear, outdoor eyewear, contrast-enhancing eyewear, chroma-enhancing eyewear, color-enhancing eyewear, color-altering eyewear, gaming eyewear, eyewear designed for another purpose, or eyewear designed for a combination of purposes. The eyewear can be provided with a unitary lens that is placed in front of both eyes or dual lenses (see, e.g., FIG. 1) with one lens placed in front of each eye when the eyewear is worn.

The phrase “disposed on” means that a first component (e.g., layer) is in direct contact with a second component. A first component “disposed on” a second component can be deposited, formed, placed, or otherwise applied directly onto the second component. In other words, if a first component is disposed on a second component, there are no components between the first component and the second component.

The phrase “disposed over” means other components (e.g., layers or substrates) may or may not be present between a first component and a second component.

### Chroma-Enhancing Dyes

The present disclosure provides chroma enhancing aminothiophene-isoxazolone dyes according to Formula I. Compounds having Formula I can have either E or Z geometry, or can be a mixture of compounds having E and Z geometry:

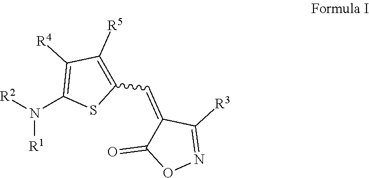

In certain aspects, R¹ and R² can each, independently, be C₁-C₆ alkyl, C₁-C₆ alkylene-OH, alkylene C(O)OR⁶, alkylene nitrile, C₁-C₆ haloalkyl, SO₂-haloalkyl, C₁-C₆ alkylene-O-alkyl, alkylene OC(O)NHR⁷, or C₃-C₆ cycloalkyl; or R¹ and R² are taken together with the nitrogen to which they are bound to form an optionally substituted 4- to 7-membered heterocycle;
R³ can be C₁-C₆alkyl, C₁-C₆ haloalkyl, C₁-C₆ alkenyl, C₁-C₆ alkynyl, C₁-C₆ alkoxy, SO₂-alkyl, SO₂-haloalkyl, SO₂-aryl, C₁-C₆ cycloalkyl, C₁-C₆ alkylene-O-alkyl, nitrile, C(O)OR⁶, optionally substituted C₆-C₁₀ aryl, or optionally substituted C₃-C₉ heteroaryl;
R⁴ and R⁵ can each, independently, be H, D, halo, C₁-C₆ alkyl, C₁-C₆ alkoxy, C₁-C₆ alkenyl, C₁-C₆ alkynyl, C₁-C₆ cycloalkyl, or C₆-C₁₀ aryl; or R⁴ and R⁵ can be taken together with the ring to which they are bound form an optionally substituted C₆-C₁₀ cycloalkyl optionally containing 1-3 heteroatoms, wherein the heteroatoms can be O or N;
R⁶ can be C₁-C₆ alkyl; and R⁷ can be H, alkyl, haloalkyl, optionally substituted phenyl, benzyl or phenylalkyl, provided that when R¹ and R² are both ethyl, R³ is not phenyl.

In another aspect, R¹ and R² can each, independently, be C₁-C₆ alkyl, alkylene C(O)OR⁶, alkylene nitrile, C₁-C₆ haloalkyl, SO₂-haloalkyl, C₁-C₆ alkylene-O-alkyl, or C₃-C₆ cycloalkyl; or R¹ and R² are taken together with the nitrogen to which they are bound to form an optionally substituted 4- to 7-membered heterocycle;
R³ can be C₁-C₆alkyl, C₁-C₆ haloalkyl, C₁-C₆ alkenyl, C₁-C₆ alkynyl, C₁-C₆ alkoxy, SO₂-haloalkyl, C₁-C₆ cycloalkyl, optionally substituted C₆-C₁₀ aryl, or optionally substituted C₃-C₉ heteroaryl;
R⁴ and R⁵ can each, independently, be H, D, halo, C₁-C₆ alkyl, C₁-C₆ alkoxy, C₁-C₆ alkenyl, C₁-C₆ alkynyl, C₁-C₆ cycloalkyl, or C₆-C₁₀ aryl, or R⁴ and R⁵ can be taken together with the ring to which they are bound form an optionally substituted C₆-C₁₀ cycloalkyl optionally containing 1-3 heteroatoms; and
R⁶ can be C₁-C₆ alkyl, provided that when R¹ and R² are both ethyl, R³ is not phenyl.

In another aspect, R¹ and R² can each, independently, be methyl, ethyl, n-propyl, n-butyl, (CH₂)ₘCO₂R⁶, (CH₂)ₘCN, (CH₂)ₘCF₃, or C₃-C₆ cycloalkyl, or R¹ and R² can be taken together with the nitrogen to which they are bound to form a 4- to 7-membered heterocycle, wherein the 4- to 7-membered heterocycle is

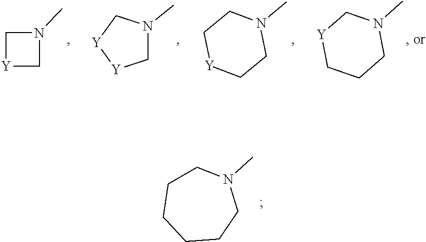

R³ can be methyl, ethyl, t-butyl, methoxy, ethoxy, CF₃, CF₂H, CH₂F, CF₂CF₃, CH₂Cl, CCl₃, CF₂Cl, CFCl₂, phenyl, pyridin-4-yl, pyridin-3-yl, pyridine-2-yl, or 4-methoxyphenyl;
R⁴ and R⁵ can each, independently, be selected from H, D, methyl, ethyl, i-propyl, t-butyl, n-butyl, halo, methoxy, ethoxy, cycloalkyl, or phenyl, or R⁴ and R⁵ can be taken together with the ring to which they are bound to form an optionally substituted C₆-C₁₀ cycloalkyl optionally containing 1-3 heteroatoms;
R⁶ can be C₁-C₆ alkyl;
each m can, independently, be 1, 2, 3, or 4; and
Y in each instance can, independently, be CH₂, O, S, SO, SO₂, CF₂, NR⁷, N—C(O)R⁷, N—SO₂—R⁷, N—C(O)OR⁷, N—C(O)N(R⁷)₂, CO, C(H)OR⁷, C(H)CN, C(H)OC(O)NHR⁷, C(H)CO₂R⁷ C(H)CF₃, C(H)-alkyl, or C(H)-aryl; and
R⁷ can be H, alkyl, haloalkyl, phenyl, benzyl or phenylalkyl, provided that when R¹ and R² are both ethyl, R³ is not phenyl.

In still further aspects, R¹ and R² can each, independently, be methyl, ethyl, or n-butyl;
R³ can be methyl, ethyl, CF₃, CF₂H, CF₂CF₃, or phenyl; and
R⁴ and R⁵ can each, independently, be H, methyl, halo, or phenyl, or R⁴ and R⁵ can be taken together with the ring to which they are bound form an optionally substituted C₆-C₁₀ cycloalkyl optionally containing 1-3 heteroatoms provided that when R¹ and R² are both ethyl, R³ is not phenyl.

In yet another aspect, R¹ and R² can be taken together with the nitrogen to which they are bound to form the 6-member heterocycle

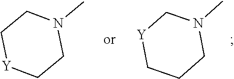

R³ can be CF₃ or phenyl;
R⁴ and R⁵ can be H; and
Y can be C(H)OH or CO.

In yet another aspect, R¹ and R² can be taken together with the nitrogen to which they are bound to form the 6-member heterocycle

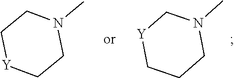

R³ can be CF₃ or phenyl;
R⁴ and R⁵ can be H;
Y can be N—C(O)R⁷, N—C(O)OR⁷ or N—SO₂—R⁷; and
R⁷ can be methyl or t-butyl.

In still another aspect, R¹ and R² can be taken together with the nitrogen to which they are bound to form the 6-member heterocycle

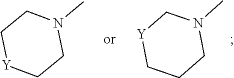

R³ can be CF₃ or phenyl;
R⁴ and R⁵ can be H; and
Y can be O, SO, or SO₂.

In another aspect, R¹ and R² can be taken together with the nitrogen to which they are attached to form the 5-member or 6-member heterocycle

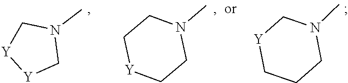

R³ can be methyl, CF₃ or phenyl; and
R⁴ and R⁵ can be H; and
Y can be CH₂.

In another aspect, R¹ and R² can each, independently, be methyl, ethyl, or n-butyl; or R¹ and R² can be taken together with the nitrogen to which they are bound to form the 4- to 7-membered heterocycle

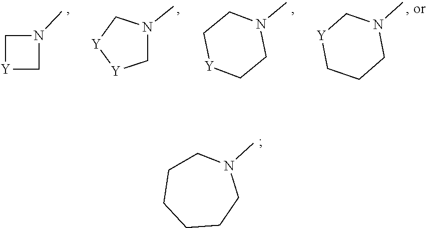

R³ can be methyl, CF₃, CF₂H, CF₂CF₃, or phenyl; and
R⁴ and R⁵ can each, independently, be H, methyl, bromo, or phenyl; and
Y in each instance can, independently, be CH₂, O, S, SO, SO₂, C(H)OH, CO, N—C(O)CH₃, N—C(O)O-t-butyl, N—C(O)OCH₃ or N—SO₂—CH₃.

In some aspects, the compound of Formula I is:
4-((5-(dimethylamino)thiophen-2-yl)methylene)-3-(trifluoromethyl)isoxazol-5(4H)-one;
4-((5-(dimethylamino)thiophen-2-yl)methylene)-3-phenylisoxazol-5(4H)-one;
4-((5-(dimethylamino)thiophen-2-yl)methylene)-3-(perfluoroethyl)isoxazol-5(4H)-one;
3-(difluoromethyl)-4-((5-(dimethylamino)thiophen-2-yl)methylene)isoxazol-5(4H)-one;
4-((5-(dimethylamino)thiophen-2-yl)methylene)-3-methylisoxazol-5(4H)-one;
4-((5-(diethylamino)thiophen-2-yl)methylene)-3-(trifluoromethyl)isoxazol-5(4H)-one;
4-((5-(diethylamino)thiophen-2-yl)methylene)-3-phenylisoxazol-5(4H)-one;
4-((5-(dibutylamino)thiophen-2-yl)methylene)-3-phenylisoxazol-5(4H)-one;
4-((5-(dimethylamino)-3-methylthiophen-2-yl)methylene)-3-(trifluoromethyl)isoxazol-5(4H)-one;
4-((5-(dimethylamino)-3-methylthiophen-2-yl)methylene)-3-phenylisoxazol-5(4H)-one;
4-((5-(piperidin-1-yl)thiophen-2-yl)methylene)-3-(trifluoromethyl)isoxazol-5(4H)-one;
3-phenyl-4-((5-(piperidin-1-yl)thiophen-2-yl)methylene)isoxazol-5(4H)-one;
3-(perfluoroethyl)-4-((5-(piperidin-1-yl)thiophen-2-yl)methylene)isoxazol-5(4H)-one;
3-(difluoromethyl)-4-((5-(piperidin-1-yl)thiophen-2-yl)methylene)isoxazol-5(4H)-one;
4-((5-(pyrrolidin-1-yl)thiophen-2-yl)methylene)-3-(trifluoromethyl)isoxazol-5(4H)-one;
3-phenyl-4-((5-(pyrrolidin-1-yl)thiophen-2-yl)methylene)isoxazol-5(4H)-one;
4-((5-morpholinothiophen-2-yl)methylene)-3-(trifluoromethyl)isoxazol-5(4H)-one;
4-((5-morpholinothiophen-2-yl)methylene)-3-phenylisoxazol-5(4H)-one;
tert-butyl-4-(5-((5-oxo-3-(trifluoromethyl)isoxazol-4(5H)-ylidene)methyl)thiophen-2-yl)piperazine-1-carboxylate;
tert-butyl-4-(5-((5-oxo-3-phenylisoxazol-4(5H)-ylidene)methyl)thiophen-2-yl)piperazine-1-carboxylate;
3-phenyl-4-((5-(piperazin-1-yl)thiophen-2-yl)methylene)isoxazol-5(4H)-one;
4-((5-(4-acetylpiperazin-1-yl)thiophen-2-yl)methylene)-3-phenylisoxazol-5(4H)-one;
4-((5-(4-(methylsulfonyl)piperazin-1-yl)thiophen-2-yl)methylene)-3-phenylisoxazol-5(4H)-one;
4-((5-(4-hydroxypiperidin-1-yl)thiophen-2-yl)methylene)-3-(trifluoromethyl)isoxazol-5(4H)-one;
4-((5-(4-hydroxypiperidin-1-yl)thiophen-2-yl)methylene)-3-phenylisoxazol-5(4H)-one;
4-((5-(4-oxopiperidin-1-yl)thiophen-2-yl)methylene)-3-(trifluoromethyl)isoxazol-5(4H)-one;
4-((5-(4-oxopiperidin-1-yl)thiophen-2-yl)methylene)-3-phenylisoxazol-5(4H)-one;
4-((5-thiomorpholinothiophen-2-yl)methylene)-3-(trifluoromethyl)isoxazol-5(4H)-one;
3-phenyl-4-((5-thiomorpholinothiophen-2-yl)methylene)isoxazol-5(4H)-one;
4-((5-(1,1-dioxidothiomorpholino)thiophen-2-yl)methylene)-3-phenylisoxazol-5(4H)-one;
4-((5-(1-oxidothiomorpholino)thiophen-2-yl)methylene)-3-phenylisoxazol-5(4H)-one;
4-((4-bromo-5-(dimethylamino)thiophen-2-yl)methylene)-3-phenylisoxazol-5(4H)-one
4-((4-bromo-5-(dimethylamino)thiophen-2-yl)methylene)-3-(trifluoromethyl)isoxazol-5(4H)-one;
4-((5-(dimethylamino)-4-phenylthiophen-2-yl)methylene)-3-(trifluoromethyl)isoxazol-5(4H)-one;
3-methyl-4-((5-(piperidin-1-yl)thiophen-2-yl)methylene)isoxazol-5(4H)-one;
4-((5-(1,1-dioxidothiomorpholino)thiophen-2-yl)methylene)-3-(perfluoroethyl)isoxazol-5(4H)-one;
4-((5-(1-oxidothiomorpholino)thiophen-2-yl)methylene)-3-(perfluoroethyl)isoxazol-5(4H)-one;
4-((5-(bis(2-methoxyethyl)amino)thiophen-2-yl)methylene)-3-(trifluoromethyl)isoxazol-5(4H)-one;
4-((5-(azepan-1-yl)thiophen-2-yl)methylene)-3-(trifluoromethyl)isoxazol-5(4H)-one;
4-((5-(azetidin-1-yl)thiophen-2-yl)methylene)-3-(trifluoromethyl)isoxazol-5(4H)-one;
4-((5-(3-hydroxypyrrolidin-1-yl)thiophen-2-yl)methylene)-3-(trifluoromethyl)isoxazol-5(4H)-one;
1-(5-((5-oxo-3-(trifluoromethyl)isoxazol-4(5H)-ylidene)methyl)thiophen-2-yl)pyrrolidin-3-yl phenylcarbamate;
4-((7-(dimethylamino)-2,3-dihydrothieno[3,4-b][1,4]dioxin-5-yl)methylene)-3-(trifluoromethyl)isoxazol-5(4H)-one;
4-((5-(dimethylamino)thiophen-2-yl)methylene)-3-ethylisoxazol-5(4H)-one;
3-(tert-butyl)-4-((5-(dimethylamino)thiophen-2-yl)methylene)isoxazol-5(4H)-one;
3-(tert-butyl)-4-((5-(piperidin-1-yl)thiophen-2-yl)methylene)isoxazol-5(4H)-one;
3-cyclopropyl-4-((5-(dimethylamino)thiophen-2-yl)methylene)isoxazol-5(4H)-one;
4-((5-(dimethylamino)thiophen-2-yl)methylene)-3-(methoxymethyl)isoxazol-5(4H)-one;
3-(butoxymethyl)-4-((5-(dimethylamino)thiophen-2-yl)methylene)isoxazol-5(4H)-one;
methyl 2-(4-((5-(dimethylamino)thiophen-2-yl)methylene)-5-oxo-4,5-dihydroisoxazol-3-yl)acetate;
butyl 2-(4-((5-(dimethylamino)thiophen-2-yl)methylene)-5-oxo-4,5-dihydroisoxazol-3-yl)acetate;
benzyl 2-(4-((5-(dimethylamino)thiophen-2-yl)methylene)-5-oxo-4,5-dihydroisoxazol-3-yl)acetate;
phenethyl 2-(4-((5-(dimethylamino)thiophen-2-yl)methylene)-5-oxo-4,5-dihydroisoxazol-3-yl)acetate;
ethyl 4-((5-(dimethylamino)thiophen-2-yl)methylene)-5-oxo-4,5-dihydroisoxazole-3-carboxylate;
butyl 4-((5-(dimethylamino)thiophen-2-yl)methylene)-5-oxo-4,5-dihydroisoxazole-3-carboxylate;
4-(4-((5-(dimethylamino)thiophen-2-yl)methylene)-5-oxo-4,5-dihydroisoxazol-3-yl)benzonitrile;
4-((5-(dimethylamino)thiophen-2-yl)methylene)-3-(4-(trifluoromethyl)phenyl)isoxazol-5(4H)-one;
4-((5-(dimethylamino)thiophen-2-yl)methylene)-3-(4-methoxyphenyl)isoxazol-5(4H)-one;
4-((5-(dimethylamino)thiophen-2-yl)methylene)-3-(pyridin-4-yl)isoxazol-5(4H)-one; or
4-((4-chloro-5-(dimethylamino)thiophen-2-yl)methylene)-3-(trifluoromethyl)isoxazol-5(4H)-one.

In some aspects, the compound of the disclosure is:
(Z)-4-((5-(dimethylamino)thiophen-2-yl)methylene)-3-(trifluoromethyl)isoxazol-5(4H)-one;
(Z)-4-((5-(dimethylamino)thiophen-2-yl)methylene)-3-phenylisoxazol-5(4H)-one;
(Z)-4-((5-(dimethylamino)thiophen-2-yl)methylene)-3-(perfluoroethyl)isoxazol-5(4H)-one;
(Z)-3-(difluoromethyl)-4-((5-(dimethylamino)thiophen-2-yl)methylene)isoxazol-5(4H)-one;
(Z)-4-((5-(dimethylamino)thiophen-2-yl)methylene)-3-methylisoxazol-5(4H)-one;
(Z)-4-((5-(diethylamino)thiophen-2-yl)methylene)-3-(trifluoromethyl)isoxazol-5(4H)-one;
(Z)-4-((5-(diethylamino)thiophen-2-yl)methylene)-3-phenylisoxazol-5(4H)-one;
(Z)-4-((5-(dibutylamino)thiophen-2-yl)methylene)-3-phenylisoxazol-5(4H)-one;
(Z)-4-((5-(dimethylamino)-3-methylthiophen-2-yl)methylene)-3-(trifluoromethyl)isoxazol-5(4H)-one;
(Z)-4-((5-(dimethylamino)-3-methylthiophen-2-yl)methylene)-3-phenylisoxazol-5(4H)-one;
(Z)-4-((5-(piperidin-1-yl)thiophen-2-yl)methylene)-3-(trifluoromethyl)isoxazol-5(4H)-one;
(Z)-3-phenyl-4-((5-(piperidin-1-yl)thiophen-2-yl)methylene)isoxazol-5(4H)-one;
(Z)-3-(perfluoroethyl)-4-((5-(piperidin-1-yl)thiophen-2-yl)methylene)isoxazol-5(4H)-one;
(Z)-3-(difluoromethyl)-4-((5-(piperidin-1-yl)thiophen-2-yl)methylene)isoxazol-5(4H)-one;
(Z)-4-((5-(pyrrolidin-1-yl)thiophen-2-yl)methylene)-3-(trifluoromethyl)isoxazol-5(4H)-one;
(Z)-3-phenyl-4-((5-(pyrrolidin-1-yl)thiophen-2-yl)methylene)isoxazol-5(4H)-one;
(Z)-4-((5-morpholinothiophen-2-yl)methylene)-3-(trifluoromethyl)isoxazol-5(4H)-one;
(Z)-4-((5-morpholinothiophen-2-yl)methylene)-3-phenylisoxazol-5(4H)-one;
tert-butyl (Z)-4-(5-((5-oxo-3-(trifluoromethyl)isoxazol-4(5H)-ylidene)methyl)thiophen-2-yl)piperazine-1-carboxylate;
tert-butyl (Z)-4-(5-((5-oxo-3-phenylisoxazol-4(5H)-ylidene)methyl)thiophen-2-yl)piperazine-1-carboxylate;
(Z)-3-phenyl-4-((5-(piperazin-1-yl)thiophen-2-yl)methylene)isoxazol-5(4H)-one;
(Z)-4-((5-(4-acetylpiperazin-1-yl)thiophen-2-yl)methylene)-3-phenylisoxazol-5(4H)-one;
(Z)-4-((5-(4-(methylsulfonyl)piperazin-1-yl)thiophen-2-yl)methylene)-3-phenylisoxazol-5(4H)-one;
(Z)-4-((5-(4-hydroxypiperidin-1-yl)thiophen-2-yl)methylene)-3-(trifluoromethyl)isoxazol-5(4H)-one;
(Z)-4-((5-(4-hydroxypiperidin-1-yl)thiophen-2-yl)methylene)-3-phenylisoxazol-5(4H)-one;
(Z)-4-((5-(4-oxopiperidin-1-yl)thiophen-2-yl)methylene)-3-(trifluoromethyl)isoxazol-5(4H)-one;
(Z)-4-((5-(4-oxopiperidin-1-yl)thiophen-2-yl)methylene)-3-phenylisoxazol-5(4H)-one;
(Z)-4-((5-thiomorpholinothiophen-2-yl)methylene)-3-(trifluoromethyl)isoxazol-5(4H)-one;
(Z)-3-phenyl-4-((5-thiomorpholinothiophen-2-yl)methylene)isoxazol-5(4H)-one;
(Z)-4-((5-(1,1-dioxidothiomorpholino)thiophen-2-yl)methylene)-3-phenylisoxazol-5(4H)-one;
(Z)-4-((5-(1-oxidothiomorpholino)thiophen-2-yl)methylene)-3-phenylisoxazol-5(4H)-one;
(Z)-4-((4-bromo-5-(dimethylamino)thiophen-2-yl)methylene)-3-phenylisoxazol-5(4H)-one
(Z)-4-((4-bromo-5-(dimethylamino)thiophen-2-yl)methylene)-3-(trifluoromethyl)isoxazol-5(4H)-one;
(Z)-4-((4-bromo-5-(dimethylamino)thiophen-2-yl)methylene)-3-phenylisoxazol-5(4H)-one;
(Z)-4-((5-(dimethylamino)-4-phenylthiophen-2-yl)methylene)-3-(trifluoromethyl)isoxazol-5(4H)-one;
(Z)-3-methyl-4-((5-(piperidin-1-yl)thiophen-2-yl)methylene)isoxazol-5(4H)-one;
(Z)-4-((5-(1,1-dioxidothiomorpholino)thiophen-2-yl)methylene)-3-(perfluoroethyl)isoxazol-5(4H)-one;
(Z)-4-((5-(1-oxidothiomorpholino)thiophen-2-yl)methylene)-3-(perfluoroethyl)isoxazol-5(4H)-one;
(Z)-4-((5-(bis(2-methoxyethyl)amino)thiophen-2-yl)methylene)-3-(trifluoromethyl)isoxazol-5(4H)-one;
(Z)-4-((5-(azepan-1-yl)thiophen-2-yl)methylene)-3-(trifluoromethyl)isoxazol-5(4H)-one;
(Z)-4-((5-(azetidin-1-yl)thiophen-2-yl)methylene)-3-(trifluoromethyl)isoxazol-5(4H)-one;
(Z)-4-((5-(3-hydroxypyrrolidin-1-yl)thiophen-2-yl)methylene)-3-(trifluoromethyl)isoxazol-5(4H)-one;
(Z)-1-(5-((5-oxo-3-(trifluoromethyl)isoxazol-4(5H)-ylidene)methyl)thiophen-2-yl)pyrrolidin-3-yl phenylcarbamate;
(Z)-4-((7-(dimethylamino)-2,3-dihydrothieno[3,4-b][1,4]dioxin-5-yl)methylene)-3-(trifluoromethyl)isoxazol-5(4H)-one;
(Z)-4-((5-(dimethylamino)thiophen-2-yl)methylene)-3-ethylisoxazol-5(4H)-one;
(Z)-3-(tert-butyl)-4-((5-(dimethylamino)thiophen-2-yl)methylene)isoxazol-5(4H)-one;
(Z)-3-(tert-butyl)-4-((5-(piperidin-1-yl)thiophen-2-yl)methylene)isoxazol-5(4H)-one;
(Z)-3-cyclopropyl-4-((5-(dimethylamino)thiophen-2-yl)methylene)isoxazol-5(4H)-one;
(Z)-4-((5-(dimethylamino)thiophen-2-yl)methylene)-3-(methoxymethyl)isoxazol-5(4H)-one;
(Z)-3-(butoxymethyl)-4-((5-(dimethylamino)thiophen-2-yl)methylene)isoxazol-5(4H)-one;
methyl (Z)-2-(4-((5-(dimethylamino)thiophen-2-yl)methylene)-5-oxo-4,5-dihydroisoxazol-3-yl)acetate;
butyl (Z)-2-(4-((5-(dimethylamino)thiophen-2-yl)methylene)-5-oxo-4,5-dihydroisoxazol-3-yl)acetate;
benzyl (Z)-2-(4-((5-(dimethylamino)thiophen-2-yl)methylene)-5-oxo-4,5-dihydroisoxazol-3-yl)acetate;
phenethyl (Z)-2-(4-((5-(dimethylamino)thiophen-2-yl)methylene)-5-oxo-4,5-dihydroisoxazol-3-yl)acetate;
ethyl (Z)-4-((5-(dimethylamino)thiophen-2-yl)methylene)-5-oxo-4,5-dihydroisoxazole-3-carboxylate;
butyl (Z)-4-((5-(dimethylamino)thiophen-2-yl)methylene)-5-oxo-4,5-dihydroisoxazole-3-carboxylate;
(Z)-4-(4-((5-(dimethylamino)thiophen-2-yl)methylene)-5-oxo-4,5-dihydroisoxazol-3-yl)benzonitrile;
(Z)-4-((5-(dimethylamino)thiophen-2-yl)methylene)-3-(4-(trifluoromethyl)phenyl)isoxazol-5(4H)-one;
(Z)-4-((5-(dimethylamino)thiophen-2-yl)methylene)-3-(4-methoxyphenyl)isoxazol-5(4H)-one;
(Z)-4-((5-(dimethylamino)thiophen-2-yl)methylene)-3-(pyridin-4-yl)isoxazol-5(4H)-one; or
(Z)-4-((4-chloro-5-(dimethylamino)thiophen-2-yl)methylene)-3-(trifluoromethyl)isoxazol-5(4H)-one.

In some aspects, the compound of the disclosure is:
(E)-4-((5-(dimethylamino)thiophen-2-yl)methylene)-3-(trifluoromethyl)isoxazol-5(4H)-one;
(E)-4-((5-(dimethylamino)thiophen-2-yl)methylene)-3-phenylisoxazol-5(4H)-one;
(E)-4-((5-(dimethylamino)thiophen-2-yl)methylene)-3-(perfluoroethyl)isoxazol-5(4H)-one;
(E)-3-(difluoromethyl)-4-((5-(dimethylamino)thiophen-2-yl)methylene)isoxazol-5(4H)-one;
(E)-4-((5-(dimethylamino)thiophen-2-yl)methylene)-3-methylisoxazol-5(4H)-one;
(E)-4-((5-(diethylamino)thiophen-2-yl)methylene)-3-(trifluoromethyl)isoxazol-5(4H)-one;
(E)-4-((5-(diethylamino)thiophen-2-yl)methylene)-3-phenylisoxazol-5(4H)-one;
(E)-4-((5-(dibutylamino)thiophen-2-yl)methylene)-3-phenylisoxazol-5(4H)-one;
(E)-4-((5-(dimethylamino)-3-methylthiophen-2-yl)methylene)-3-(trifluoromethyl)isoxazol-5(4H)-one;
(E)-4-((5-(dimethylamino)-3-methylthiophen-2-yl)methylene)-3-phenylisoxazol-5(4H)-one;
(E)-4-((5-(piperidin-1-yl)thiophen-2-yl)methylene)-3-(trifluoromethyl)isoxazol-5(4H)-one;
(E)-3-phenyl-4-((5-(piperidin-1-yl)thiophen-2-yl)methylene)isoxazol-5(4H)-one;
(E)-3-(perfluoroethyl)-4-((5-(piperidin-1-yl)thiophen-2-yl)methylene)isoxazol-5(4H)-one;
(E)-3-(difluoromethyl)-4-((5-(piperidin-1-yl)thiophen-2-yl)methylene)isoxazol-5(4H)-one;
(E)-4-((5-(pyrrolidin-1-yl)thiophen-2-yl)methylene)-3-(trifluoromethyl)isoxazol-5(4H)-one;
(E)-3-phenyl-4-((5-(pyrrolidin-1-yl)thiophen-2-yl)methylene)isoxazol-5(4H)-one;
(E)-4-((5-morpholinothiophen-2-yl)methylene)-3-(trifluoromethyl)isoxazol-5(4H)-one;
(E)-4-((5-morpholinothiophen-2-yl)methylene)-3-phenylisoxazol-5(4H)-one;
tert-butyl (E)-4-(5-((5-oxo-3-(trifluoromethyl)isoxazol-4(5H)-ylidene)methyl)thiophen-2-yl)piperazine-1-carboxylate;
tert-butyl (E)-4-(5-((5-oxo-3-phenylisoxazol-4(5H)-ylidene)methyl)thiophen-2-yl)piperazine-1-carboxylate;
(E)-3-phenyl-4-((5-(piperazin-1-yl)thiophen-2-yl)methylene)isoxazol-5(4H)-one;
(E)-4-((5-(4-acetylpiperazin-1-yl)thiophen-2-yl)methylene)-3-phenylisoxazol-5(4H)-one;
(E)-4-((5-(4-(methylsulfonyl)piperazin-1-yl)thiophen-2-yl)methylene)-3-phenylisoxazol-5(4H)-one;
(E)-4-((5-(4-hydroxypiperidin-1-yl)thiophen-2-yl)methylene)-3-(trifluoromethyl)isoxazol-5(4H)-one;
(E)-4-((5-(4-hydroxypiperidin-1-yl)thiophen-2-yl)methylene)-3-phenylisoxazol-5(4H)-one;
(E)-4-((5-(4-oxopiperidin-1-yl)thiophen-2-yl)methylene)-3-(trifluoromethyl)isoxazol-5(4H)-one;
(E)-4-((5-(4-oxopiperidin-1-yl)thiophen-2-yl)methylene)-3-phenylisoxazol-5(4H)-one;
(E)-4-((5-thiomorpholinothiophen-2-yl)methylene)-3-(trifluoromethyl)isoxazol-5(4H)-one;
(E)-3-phenyl-4-((5-thiomorpholinothiophen-2-yl)methylene)isoxazol-5(4H)-one;
(E)-4-((5-(1,1-dioxidothiomorpholino)thiophen-2-yl)methylene)-3-phenylisoxazol-5(4H)-one;
(E)-4-((5-(1-oxidothiomorpholino)thiophen-2-yl)methylene)-3-phenylisoxazol-5(4H)-one;
(E)-4-((4-bromo-5-(dimethylamino)thiophen-2-yl)methylene)-3-phenylisoxazol-5(4H)-one
(E)-4-((4-bromo-5-(dimethylamino)thiophen-2-yl)methylene)-3-(trifluoromethyl)isoxazol-5(4H)-one;
(E)-4-((5-(dimethylamino)-4-phenylthiophen-2-yl)methylene)-3-(trifluoromethyl)isoxazol-5(4H)-one;
(E)-3-methyl-4-((5-(piperidin-1-yl)thiophen-2-yl)methylene)isoxazol-5(4H)-one;
(E)-4-((5-(1,1-dioxidothiomorpholino)thiophen-2-yl)methylene)-3-(perfluoroethyl)isoxazol-5(4H)-one;
(E)-4-((5-(1-oxidothiomorpholino)thiophen-2-yl)methylene)-3-(perfluoroethyl)isoxazol-5(4H)-one;
(E)-4-((5-(bis(2-methoxyethyl)amino)thiophen-2-yl)methylene)-3-(trifluoromethyl)isoxazol-5(4H)-one;
(E)-4-((5-(azepan-1-yl)thiophen-2-yl)methylene)-3-(trifluoromethyl)isoxazol-5(4H)-one;
(E)-4-((5-(azetidin-1-yl)thiophen-2-yl)methylene)-3-(trifluoromethyl)isoxazol-5(4H)-one;
(E)-4-((5-(3-hydroxypyrrolidin-1-yl)thiophen-2-yl)methylene)-3-(trifluoromethyl)isoxazol-5(4H)-one;
(E)-1-(5-((5-oxo-3-(trifluoromethyl)isoxazol-4(5H)-ylidene)methyl)thiophen-2-yl)pyrrolidin-3-yl phenylcarbamate;
(E)-4-((7-(dimethylamino)-2,3-dihydrothieno[3,4-b][1,4]dioxin-5-yl)methylene)-3-(trifluoromethyl)isoxazol-5(4H)-one;
(E)-4-((5-(dimethylamino)thiophen-2-yl)methylene)-3-ethylisoxazol-5(4H)-one;
(E)-3-(tert-butyl)-4-((5-(dimethylamino)thiophen-2-yl)methylene)isoxazol-5(4H)-one;
(E)-3-(tert-butyl)-4-((5-(piperidin-1-yl)thiophen-2-yl)methylene)isoxazol-5(4H)-one;
(E)-3-cyclopropyl-4-((5-(dimethylamino)thiophen-2-yl)methylene)isoxazol-5(4H)-one;
(E)-4-((5-(dimethylamino)thiophen-2-yl)methylene)-3-(methoxymethyl)isoxazol-5(4H)-one;
(E)-3-(butoxymethyl)-4-((5-(dimethylamino)thiophen-2-yl)methylene)isoxazol-5(4H)-one;
methyl (E)-2-(4-((5-(dimethylamino)thiophen-2-yl)methylene)-5-oxo-4,5-dihydroisoxazol-3-yl)acetate;
butyl (E)-2-(4-((5-(dimethylamino)thiophen-2-yl)methylene)-5-oxo-4,5-dihydroisoxazol-3-yl)acetate;
benzyl (E)-2-(4-((5-(dimethylamino)thiophen-2-yl)methylene)-5-oxo-4,5-dihydroisoxazol-3-yl)acetate;
phenethyl (E)-2-(4-((5-(dimethylamino)thiophen-2-yl)methylene)-5-oxo-4,5-dihydroisoxazol-3-yl)acetate;
ethyl (E)-4-((5-(dimethylamino)thiophen-2-yl)methylene)-5-oxo-4,5-dihydroisoxazole-3-carboxylate;
butyl (E)-4-((5-(dimethylamino)thiophen-2-yl)methylene)-5-oxo-4,5-dihydroisoxazole-3-carboxylate;
(E)-4-(4-((5-(dimethylamino)thiophen-2-yl)methylene)-5-oxo-4,5-dihydroisoxazol-3-yl)benzonitrile;
(E)-4-((5-(dimethylamino)thiophen-2-yl)methylene)-3-(4-(trifluoromethyl)phenyl)isoxazol-5(4H)-one;
(E)-4-((5-(dimethylamino)thiophen-2-yl)methylene)-3-(4-methoxyphenyl)isoxazol-5(4H)-one;
(E)-4-((5-(dimethylamino)thiophen-2-yl)methylene)-3-(pyridin-4-yl)isoxazol-5(4H)-one; or
(E)-4-((4-chloro-5-(dimethylamino)thiophen-2-yl)methylene)-3-(trifluoromethyl)isoxazol-5(4H)-one.

In certain aspects, the chroma-enhancing dye of Formula I can be a compound as set forth in Table 1, below.

TABLE 1
Representative compounds of Formula I

| Cmpd | | |
|---|---|---|
| No. | Cmpd Name | Structure |
| 1 | 4-((5-(dimethylamino)thiophen-2- yl)methylene)-3- (trifluoromethyl)isoxazol-5(4H)-one | 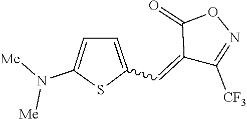 [SMILES: CN(C)c1ccc(C=C2C(=O)ON=C2C(F)(F)F)s1] |
| 2 | 4-((5-(dimethylamino)thiophen-2- yl)methylene)-3-phenylisoxazol- 5(4H)-one | 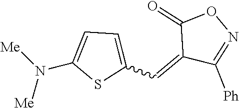 [SMILES: CN(C)c1ccc(C=C2C(=O)ON=C2c2ccccc2)s1] |
| 3 | 4-((5-(dimethylamino)thiophen-2- yl)methylene)-3- (perfluoroethyl)isoxazol-5(4H)-one | 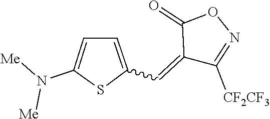 |
| 4 | 3-(difluoromethyl)-4-((5- (dimethylamino)thiophen-2- yl)methylene)isoxazol-5(4H)-one | 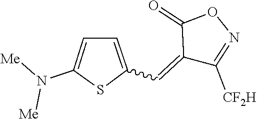 [SMILES: CN(C)c1ccc(C=C2C(=O)ON=C2C(F)F)s1] |
| 5 | 4-((5-(dimethylamino)thiophen-2- yl)methylene)-3-methylisoxazol- 5(4H)-one | 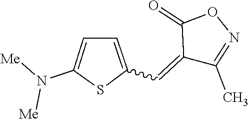 [SMILES: CC1=NOC(=O)C1=Cc1ccc(N(C)C)s1] |
| 6 | 4-((5-(diethylamino)thiophen-2- yl)methylene)-3- (trifluoromethyl)isoxazol-5(4H)-one | 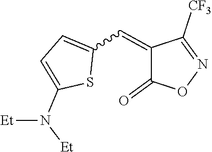 [SMILES: CCN(CC)c1ccc(C=C2C(=O)ON=C2C(F)(F)F)s1] |
| 7 | 4-((5-(diethylamino)thiophen-2- yl)methylene)-3-phenylisoxazol- 5(4H)-one | 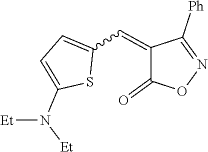 [SMILES: CCN(CC)c1ccc(C=C2C(=O)ON=C2c2ccccc2)s1] |
| 8 | 4-((5-(dibutylamino)thiophen-2- yl)methylene)-3-phenylisoxazol- 5(4H)-one | 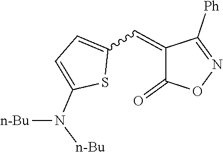 |
| 9 | 4-((5-(dimethylamino)-3- methylthiophen-2-yl)methylene)-3- (trifluoromethyl)isoxazol-5(4H)-one | 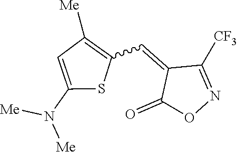 [SMILES: Cc1cc(N(C)C)sc1C=C1C(=O)ON=C1C(F)(F)F] |
| 10 | 4-((5-(dimethylamino)-3- methylthiophen-2-yl)methylene)-3- phenylisoxazol-5(4H)-one | 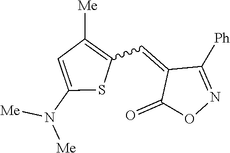 [SMILES: Cc1cc(N(C)C)sc1C=C1C(=O)ON=C1c1ccccc1] |
| 11 | 4-((5-(piperidin-1-yl)thiophen-2- yl)methylene)-3- (trifluoromethyl)isoxazol-5(4H)-one | 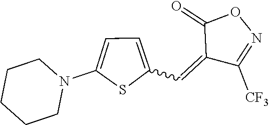 [SMILES: O=C1ON=C(C(F)(F)F)C1=Cc1ccc(N2CCCCC2)s1] |
| 12 | 3-phenyl-4-((5-(piperidin-1- yl)thiophen-2- yl)methylene)isoxazol-5(4H)-one | 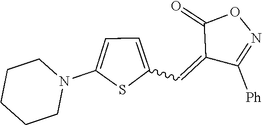 [SMILES: O=C1ON=C(c2ccccc2)C1=Cc1ccc(N2CCCCC2)s1] |
| 13 | 3-(perfluoroethyl)-4-((5-(piperidin- 1-yl)thiophen-2- yl)methylene)isoxazol-5(4H)-one | 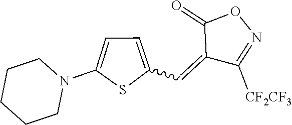 |
| 14 | 3-(difluoromethyl)-4-((5-(piperidin- 1-yl)thiophen-2- yl)methylene)isoxazol-5(4H)-one | 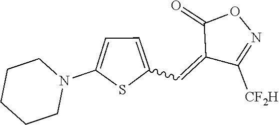 [SMILES: O=C1ON=C(C(F)F)C1=Cc1ccc(N2CCCCC2)s1] |
| 15 | 4-((5-(pyrrolidin-1-yl)thiophen-2- yl)methylene)-3- (trifluoromethyl)isoxazol-5(4H)-one | 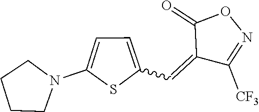 [SMILES: O=C1ON=C(C(F)(F)F)C1=Cc1ccc(N2CCCC2)s1] |
| 16 | 3-phenyl-4-((5-(pyrrolidin-1- yl)thiophen-2- yl)methylene)isoxazol-5(4H)-one | 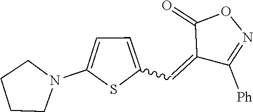 [SMILES: O=C1ON=C(c2ccccc2)C1=Cc1ccc(N2CCCC2)s1] |
| 17 | 4-((5-morpholinothiophen-2- yl)methylene)-3- (trifluoromethyl)isoxazol-5(4H)-one | 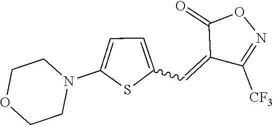 [SMILES: O=C1ON=C(C(F)(F)F)C1=Cc1ccc(N2CCOCC2)s1] |
| 18 | 4-((5-morpholinothiophen-2- yl)methylene)-3-phenylisoxazol- 5(4H)-one | 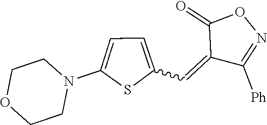 [SMILES: O=C1ON=C(c2ccccc2)C1=Cc1ccc(N2CCOCC2)s1] |
| 19 | tert-butyl-4-(5-((5-oxo-3- (trifluoromethyl)isoxazol-4(5H)- ylidene)methyl)thiophen-2- yl)piperazine-1-carboxylate | 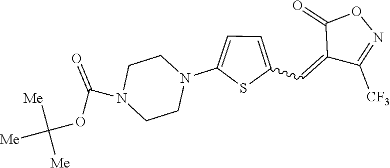 [SMILES: CC(C)(C)OC(=O)N1CCN(c2ccc(C=C3C(=O)ON=C3C(F)(F)F)s2)CC1] |
| 20 | tert-butyl-4-(5-((5-oxo-3- phenylisoxazol-4(5H)- ylidene)methyl)thiophen-2- yl)piperazine-1-carboxylate | 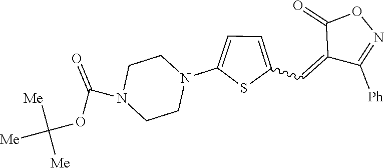 [SMILES: CC(C)(C)OC(=O)N1CCN(c2ccc(C=C3C(=O)ON=C3c3ccccc3)s2)CC1] |
| 21 | 3-phenyl-4-((5-(piperazin-1- yl)thiophen-2- yl)methylene)isoxazol-5(4H)-one | 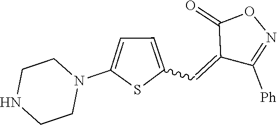 [SMILES: O=C1ON=C(c2ccccc2)C1=Cc1ccc(N2CCNCC2)s1] |
| 22 | 4-((5-(4-acetylpiperazin-1- yl)thiophen-2-yl)methylene)-3- phenylisoxazol-5(4H)-one | 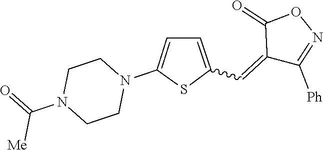 [SMILES: CC(=O)N1CCN(c2ccc(C=C3C(=O)ON=C3c3ccccc3)s2)CC1] |
| 23 | 4-((5-(4-(methylsulfonyl)piperazin- 1-yl)thiophen-2-yl)methylene)-3- phenylisoxazol-5(4H)-one | 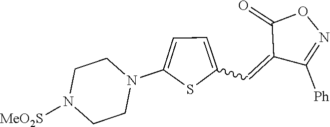 [SMILES: CS(=O)(=O)N1CCN(c2ccc(C=C3C(=O)ON=C3c3ccccc3)s2)CC1] |
| 24 | 4-((5-(4-hydroxypiperidin-1- yl)thiophen-2-yl)methylene)-3- (trifluoromethyl)isoxazol-5(4H)-one | 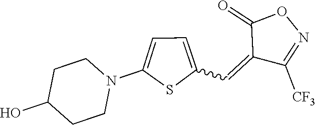 [SMILES: O=C1ON=C(C(F)(F)F)C1=Cc1ccc(N2CCC(O)CC2)s1] |
| 25 | 4-((5-(4-hydroxypiperidin-1- yl)thiophen-2-yl)methylene)-3- phenylisoxazol-5(4H)-one | 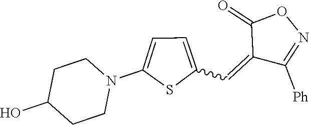 [SMILES: O=C1ON=C(c2ccccc2)C1=Cc1ccc(N2CCC(O)CC2)s1] |
| 26 | 4-((5-(4-oxopiperidin-1- yl)thiophen-2-yl)methylene)-3- (trifluoromethyl)isoxazol-5(4H)-one | 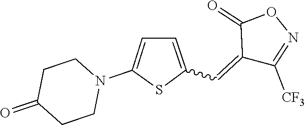 [SMILES: O=C1CCN(c2ccc(C=C3C(=O)ON=C3C(F)(F)F)s2)CC1] |
| 27 | 4-((5-(4-oxopiperidin-1- yl)thiophen-2-yl)methylene)-3- phenylisoxazol-5(4H)-one | 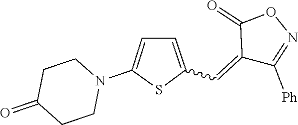 [SMILES: O=C1CCN(c2ccc(C=C3C(=O)ON=C3c3ccccc3)s2)CC1] |
| 28 | 4-((5-thiomorpholinothiophen-2- yl)methylene)-3- (trifluoromethyl)isoxazol-5(4H)-one | 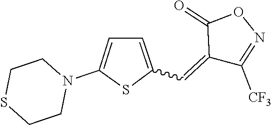 [SMILES: O=C1ON=C(C(F)(F)F)C1=Cc1ccc(N2CCSCC2)s1] |
| 29 | 3-phenyl-4-((5- thiomorpholinothiophen-2- yl)methylene)isoxazol-5(4H)-one | 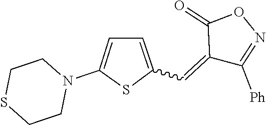 [SMILES: O=C1ON=C(c2ccccc2)C1=Cc1ccc(N2CCSCC2)s1] |
| 30 | 4-((5-(1,1- dioxidothiomorpholino)thiophen-2- yl)methylene)-3-phenylisoxazol- 5(4H)-one | 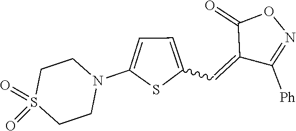 [SMILES: O=C1ON=C(c2ccccc2)C1=Cc1ccc(N2CCS(=O)(=O)CC2)s1] |
| 31 | 4-((5-(1- oxidothiomorpholino)thiophen-2- yl)methylene)-3-phenylisoxazol- 5(4H)-one | 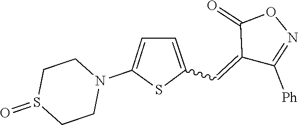 [SMILES: O=C1ON=C(c2ccccc2)C1=Cc1ccc(N2CCS(=O)CC2)s1] |
| 32 | 4-((4-bromo-5- (dimethylamino)thiophen-2- yl)methylene)-3-phenylisoxazol- 5(4H)-one | 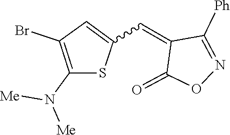 [SMILES: CN(C)c1sc(C=C2C(=O)ON=C2c2ccccc2)cc1Br] |
| 33 | 4-((4-bromo-5- (dimethylamino)thiophen-2- yl)methylene)-3- (trifluoromethyl)isoxazol-5(4H)-one | 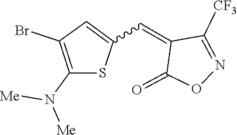 [SMILES: CN(C)c1sc(C=C2C(=O)ON=C2C(F)(F)F)cc1Br] |
| 34 | 4-((5-(dimethylamino)-4- phenylthiophen-2-yl)methylene)-3- (trifluoromethyl)isoxazol-5(4H)-one | 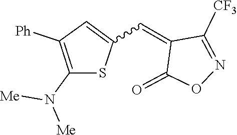 [SMILES: CN(C)c1sc(C=C2C(=O)ON=C2C(F)(F)F)cc1-c1ccccc1] |
| 35 | 3-methyl-4-((5-(piperidin-1- yl)thiophen-2- yl)methylene)isoxazol-5(4H)-one | 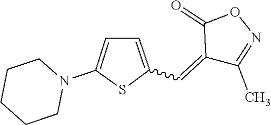 [SMILES: CC1=NOC(=O)C1=Cc1ccc(N2CCCCC2)s1] |
| 36 | 4-((5-(1,1- dioxidothiomorpholino)thiophen-2- yl)methylene)-3- (perfluoroethyl)isoxazol-5(4H)-one | 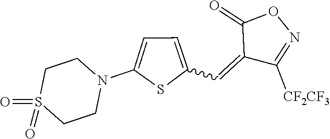 |
| 37 | 4-((5-(1- oxidothiomorpholino)thiophen-2- yl)methylene)-3- (perfluoroethyl)isoxazol-5(4H)-one | 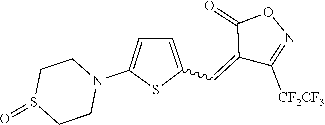 |
| 38 | 4-((5-(bis(2- methoxyethyl)amino)thiophen-2- yl)methylene)-3- (trifluoromethyl)isoxazol-5(4H)-one | 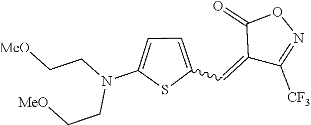 [SMILES: COCCN(CCOC)c1ccc(C=C2C(=O)ON=C2C(F)(F)F)s1] |
| 39 | 4-((5-(azepan-1-yl)thiophen-2- yl)methylene)-3- (trifluoromethyl)isoxazol-5(4H)-one | 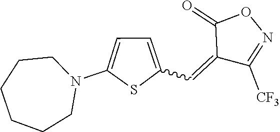 [SMILES: O=C1ON=C(C(F)(F)F)C1=Cc1ccc(N2CCCCCC2)s1] |
| 40 | 4-((5-(azetidin-1-yl)thiophen-2- yl)methylene)-3- (trifluoromethyl)isoxazol-5(4H)-one | 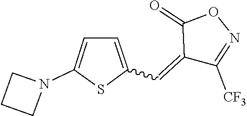 [SMILES: O=C1ON=C(C(F)(F)F)C1=Cc1ccc(N2CCC2)s1] |
| 41 | 4-((5-(3-hydroxypyrrolidin-1- yl)thiophen-2-yl)methylene)-3- (trifluoromethyl)isoxazol-5(4H)-one | 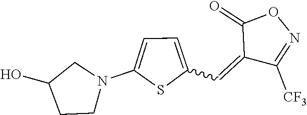 [SMILES: O=C1ON=C(C(F)(F)F)C1=Cc1ccc(N2CCC(O)C2)s1] |
| 42 | 1-(5-((5-oxo-3- (trifluoromethyl)isoxazol-4(5H)- ylidene)methyl)thiophen-2- yl)pyrrolidin-3-yl phenylcarbamate | 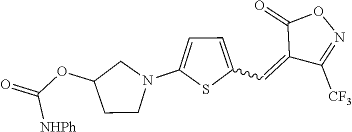 |
| 43 | 4-((7-(dimethylamino)-2,3- dihydrothieno[3,4-b][1,4]dioxin-5- yl)methylene)-3- (trifluoromethyl)isoxazol-5(4H)-one | 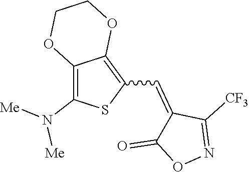 [SMILES: CN(C)c1sc(C=C2C(=O)ON=C2C(F)(F)F)c2c1OCCO2] |
| 44 | 4-((5-(dimethylamino)thiophen-2- yl)methylene)-3-ethylisoxazol- 5(4H)-one | 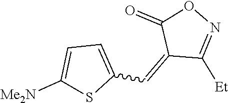 [SMILES: CCC1=NOC(=O)C1=Cc1ccc(N(C)C)s1] |
| 45 | 3-(tert-butyl)-4-((5- (dimethylamino)thiophen-2- yl)methylene)isoxazol-5(4H)-one | 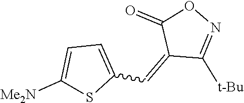 [SMILES: CN(C)c1ccc(C=C2C(=O)ON=C2C(C)(C)C)s1] |
| 46 | 3-(tert-butyl)-4-((5-(piperidin-1- yl)thiophen-2- yl)methylene)isoxazol-5(4H)-one | 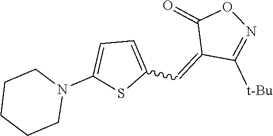 [SMILES: CC(C)(C)C1=NOC(=O)C1=Cc1ccc(N2CCCCC2)s1] |
| 47 | 3-cyclopropyl-4-((5- (dimethylamino)thiophen-2- yl)methylene)isoxazol-5(4H)-one | 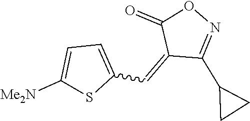 [SMILES: CN(C)c1ccc(C=C2C(=O)ON=C2C2CC2)s1] |
| 48 | 4-((5-(dimethylamino)thiophen-2- yl)methylene)-3- (methoxymethyl)isoxazol-5(4H)- one | 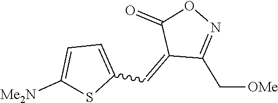 [SMILES: COCC1=NOC(=O)C1=Cc1ccc(N(C)C)s1] |
| 49 | 3-(butoxymethyl)-4-((5- (dimethylamino)thiophen-2- yl)methylene)isoxazol-5(4H)-one | 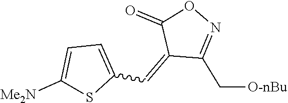 |
| 50 | methyl 2-(4-((5- (dimethylamino)thiophen-2- yl)methylene)-5-oxo-4,5- dihydroisoxazol-3-yl)acetate | 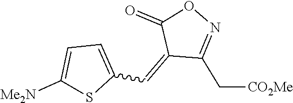 [SMILES: COC(=O)CC1=NOC(=O)C1=Cc1ccc(N(C)C)s1] |
| 51 | butyl 2-(4-((5- (dimethylamino)thiophen-2- yl)methylene)-5-oxo-4,5- dihydroisoxazol-3-yl)acetate | 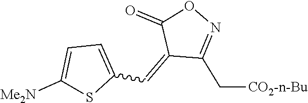 |
| 52 | benzyl 2-(4-((5- (dimethylamino)thiophen-2- yl)methylene)-5-oxo-4,5- dihydroisoxazol-3-yl)acetate | 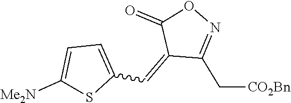 [SMILES: CN(C)c1ccc(C=C2C(=O)ON=C2CC(=O)OCc2ccccc2)s1] |
| 53 | phenethyl 2-(4-((5- (dimethylamino)thiophen-2- yl)methylene)-5-oxo-4,5- dihydroisoxazol-3-yl)acetate | 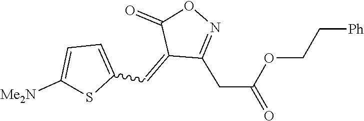 [SMILES: CN(C)c1ccc(C=C2C(=O)ON=C2CC(=O)OCCc2ccccc2)s1] |
| 54 | ethyl 4-((5- (dimethylamino)thiophen-2- yl)methylene)-5-oxo-4,5- dihydroisoxazole-3-carboxylate | 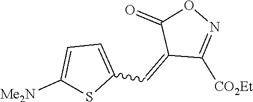 [SMILES: CCOC(=O)C1=NOC(=O)C1=Cc1ccc(N(C)C)s1] |
| 55 | butyl 4-((5- (dimethylamino)thiophen-2- yl)methylene)-5-oxo-4,5- dihydroisoxazole-3-carboxylate | 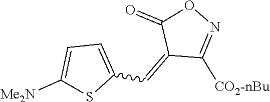 |
| 56 | 4-(4-((5-(dimethylamino)thiophen- 2-yl)methylene)-5-oxo-4,5- dihydroisoxazol-3-yl)benzonitrile | 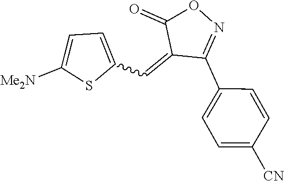 [SMILES: CN(C)c1ccc(C=C2C(=O)ON=C2c2ccc(C#N)cc2)s1] |
| 57 | 4-((5-(dimethylamino)thiophen-2- yl)methylene)-3-(4- (trifluoromethyl)phenyl)isoxazol- 5(4H)-one | 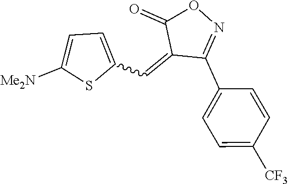 [SMILES: CN(C)c1ccc(C=C2C(=O)ON=C2c2ccc(C(F)(F)F)cc2)s1] |
| 58 | 4-((5-(dimethylamino)thiophen-2- yl)methylene)-3-(4- methoxyphenyl)isoxazol-5(4H)-one | 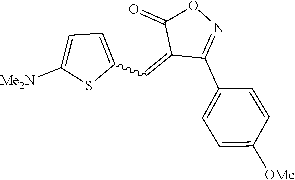 [SMILES: COc1ccc(C2=NOC(=O)C2=Cc2ccc(N(C)C)s2)cc1] |
| 59 | 4-((5-(dimethylamino)thiophen-2- yl)methylene)-3-(pyridin-4- yl)isoxazol-5(4H)-one | 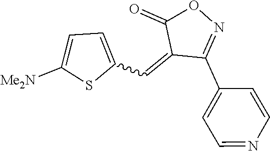 [SMILES: CN(C)c1ccc(C=C2C(=O)ON=C2c2ccncc2)s1] |
| 60 | 4-((4-chloro-5- (dimethylamino)thiophen-2- yl)methylene)-3- (trifluoromethyl)isoxazol-5(4H)-one | 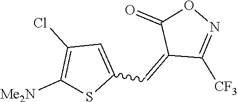 [SMILES: CN(C)c1sc(C=C2C(=O)ON=C2C(F)(F)F)cc1Cl] |

In other aspects, the chroma-enhancing dye of Formula I can be a compound as set forth in Table 2, below.

TABLE 2
Additional Non-limiting Aminothiophne-Isoxazolone Dyes for Chroma-Enhancement.

| Cmpd | | |
|---|---|---|
| No. | Cmpd Name | Structure |
| 61 | 4-((5-(dibutylamino)thiophen-2- yl)methylene)-3- (trifluoromethyl)isoxazol-5(4H)-one | 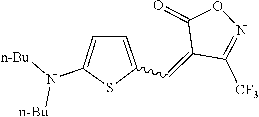 |
| 62 | 3-(chloromethyl)-4-((5- (dimethylamino)thiophen-2- yl)methylene)isoxazol-5(4H)-one | 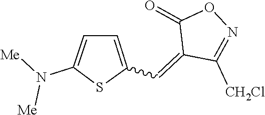 |
| 63 | 4-((5-(dimethylamino)thiophen-2- yl)methylene)-3- (fluoromethyl)isoxazol-5(4H)-one | 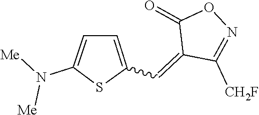 |
| 64 | 4-((5-(dimethylamino)thiophen-2- yl)methylene)-3- (trichloromethyl)isoxazol-5(4H)- one | 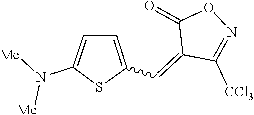 |
| 65 | 4-((5-(dimethylamino)thiophen-2- yl)methylene)-5-oxo-4,5- dihydroisoxazole-3-carbonitrile | 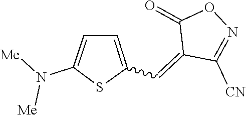 [SMILES: CN(C)c1ccc(C=C2C(=O)ON=C2C#N)s1] |
| 66 | 4-((5-(dimethylamino)thiophen-2- yl)methylene)-3- (methylsulfonyl)isoxazol-5(4H)-one | 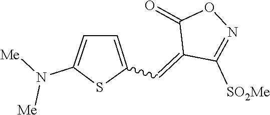 [SMILES: CN(C)c1ccc(C=C2C(=O)ON=C2S(C)(=O)=O)s1] |
| 67 | 4-((5-(dimethylamino)thiophen-2- yl)methylene)-3- (phenylsulfonyl)isoxazol-5(4H)-one | 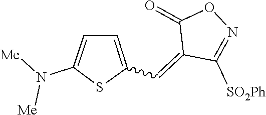 |
| 68 | 4-((5-(dimethylamino)thiophen-2- yl)methylene)-3- ((trifluoromethyl)sulfonyl)isoxazol- 5(4H)-one | 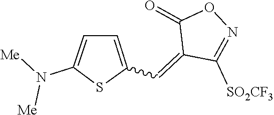 |
| 69 | 4-((5-(dimethylamino)-3,4- dimethylthiophen-2-yl)methylene)- 3-(trifluoromethyl)isoxazol-5(4H)- one | 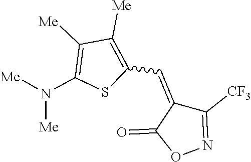 [SMILES: Cc1c(C=C2C(=O)ON=C2C(F)(F)F)sc(N(C)C)c1C] |
| 70 | 4-((3-(dimethylamino)-4,5,6,7- tetrahydrobenzo[c]thiophen-1- yl)methylene)-3- (trifluoromethyl)isoxazol-5(4H)-one | 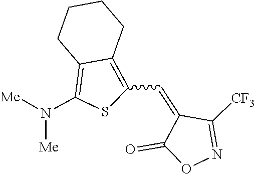 [SMILES: CN(C)c1sc(C=C2C(=O)ON=C2C(F)(F)F)c2c1CCCC2] |
| 71 | 4-((5-(dimethylamino)-3- methoxythiophen-2-yl)methylene)- 3-(trifluoromethyl)isoxazol-5(4H)- one | 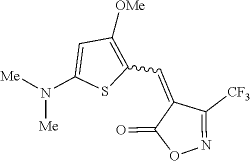 [SMILES: COc1cc(N(C)C)sc1C=C1C(=O)ON=C1C(F)(F)F] |
| 72 | 4-((5-((2- methoxyethyl)(methyl)amino)thio- phen-2-yl)methylene)-3- (trifluoromethyl)isoxazol-5(4H)-one | 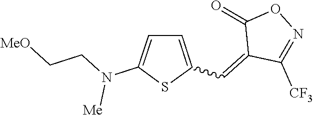 [SMILES: COCCN(C)c1ccc(C=C2C(=O)ON=C2C(F)(F)F)s1] |
| 73 | 4-((5-(dimethylamino)-3- phenylthiophen-2-yl)methylene)-3- (trifluoromethyl)isoxazol-5(4H)-one | 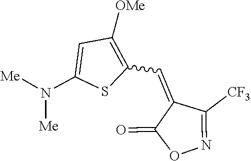 [SMILES: COc1cc(N(C)C)sc1C=C1C(=O)ON=C1C(F)(F)F] |
| 74 | 3-(methyl(5-((5-oxo-3- (trifluoromethyl)isoxazol-4(5H)- ylidene)methyl)thiophen-2- yl)amino)propanenitrile | 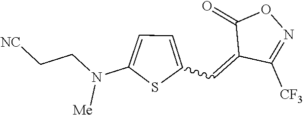 [SMILES: CN(CCC#N)c1ccc(C=C2C(=O)ON=C2C(F)(F)F)s1] |
| 75 | ethyl 3-(methyl(5-((5-oxo-3- (trifluoromethyl)isoxazol-4(5H)- ylidene)methyl)thiophen-2- yl)amino)propanoate | 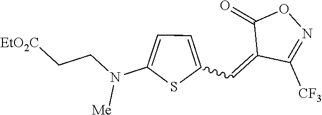 [SMILES: CCOC(=O)CCN(C)c1ccc(C=C2C(=O)ON=C2C(F)(F)F)s1] |
| 76 | 3,3′-((5-((5-oxo-3- (trifluoromethyl)isoxazol-4(5H)- ylidene)methyl)thiophen-2- yl)azanediyl)dipropanenitrile | 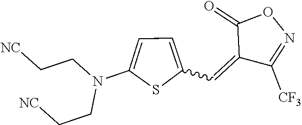 [SMILES: N#CCCN(CCC#N)c1ccc(C=C2C(=O)ON=C2C(F)(F)F)s1] |
| 77 | diethyl 3,3′-((5-((5-oxo-3- (trifluoromethyl)isoxazol-4(5H)- ylidene)methyl)thiophen-2- yl)azanediyl)dipropionate | 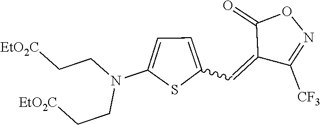 [SMILES: CCOC(=O)CCN(CCC(=O)OCC)c1ccc(C=C2C(=O)ON=C2C(F)(F)F)s1] |
| 78 | diethyl 2,2′-((5-((5-oxo-3- (trifluoromethyl)isoxazol-4(5H)- ylidene)methyl)thiophen-2- yl)azanediyl)diacetate | 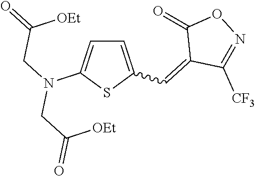 [SMILES: CCOC(=O)CN(CC(=O)OCC)c1ccc(C=C2C(=O)ON=C2C(F)(F)F)s1] |
| 79 | benzyl 4-(5-((5-oxo-3- (trifluoromethyl)isoxazol-4(5H)- ylidene)methyl)thiophen-2- yl)piperazine-1-carboxylate | 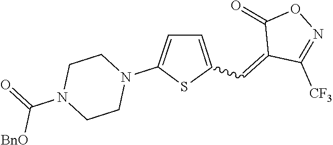 [SMILES: O=C1ON=C(C(F)(F)F)C1=Cc1ccc(N2CCN(C(=O)OCc3ccccc3)CC2)s1] |
| 80 | 4-((5-(4-acetylpiperazin-1- yl)thiophen-2-yl)methylene)-3- (trifluoromethyl)isoxazol-5(4H)-one | 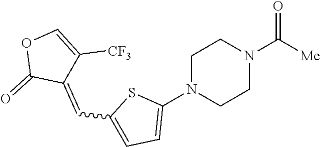 [SMILES: CC(=O)N1CCN(c2ccc(C=C3C(=O)OC=C3C(F)(F)F)s2)CC1] |
| 81 | 4-((5-(4-(methylsulfonyl)piperazin- 1-yl)thiophen-2-yl)methylene)-3- (trifluoromethyl)isoxazol-5(4H)-one | 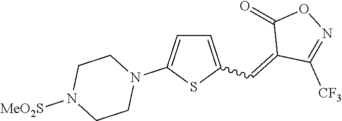 [SMILES: CS(=O)(=O)N1CCN(c2ccc(C=C3C(=O)ON=C3C(F)(F)F)s2)CC1] |
| 82 | 4-((5-(piperazin-1-yl)thiophen-2- yl)methylene)-3- (trifluoromethyl)isoxazol-5(4H)-one | 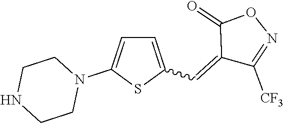 [SMILES: O=C1ON=C(C(F)(F)F)C1=Cc1ccc(N2CCNCC2)s1] |
| 83 | 4-((5-(4-methylpiperazin-1- yl)thiophen-2-yl)methylene)-3- (trifluoromethyl)isoxazol-5(4H)-one | 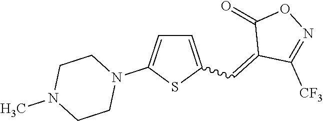 [SMILES: CN1CCN(c2ccc(C=C3C(=O)ON=C3C(F)(F)F)s2)CC1] |
| 84 | N,N-dimethyl-4-(5-((5-oxo-3- (trifluoromethyl)isoxazol-4(5H)- ylidene)methyl)thiophen-2- yl)piperazine-1-carboxamide | 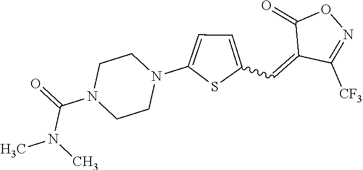 [SMILES: CN(C)C(=O)N1CCN(c2ccc(C=C3C(=O)ON=C3C(F)(F)F)s2)CC1] |
| 85 | 4-(5-((5-oxo-3- (trifluoromethyl)isoxazol-4(5H)- ylidene)methyl)thiophen-2-yl)-N- phenylpiperazine-1-carboxamide | 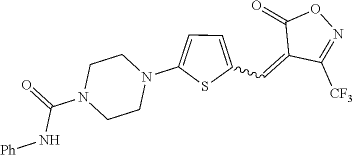 [SMILES: O=C1ON=C(C(F)(F)F)C1=Cc1ccc(N2CCN(C(=O)Nc3ccccc3)CC2)s1] |
| 86 | 3-(methyl(5-((5-oxo-3- phenylisoxazol-4(5H)- ylidene)methyl)thiophen-2- yl)amino)propanenitrile | 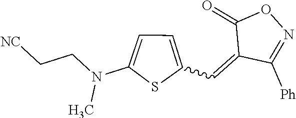 [SMILES: CN(CCC#N)c1ccc(C=C2C(=O)ON=C2c2ccccc2)s1] |
| 87 | diethyl 2,2′-((5-((5-oxo-3- phenylisoxazol-4(5H)- ylidene)methyl)thiophen-2- yl)azanediyl)diacetate | 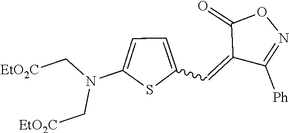 [SMILES: CCOC(=O)CN(CC(=O)OCC)c1ccc(C=C2C(=O)ON=C2c2ccccc2)s1] |
| 88 | 3-(difluoromethyl)-4-((5-(1,1- dioxidothiomorpholino)thiophen-2- yl)methylene)isoxazol-5(4H)-one | 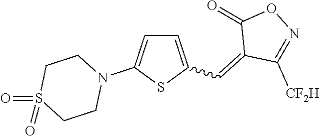 [SMILES: O=C1ON=C(C(F)F)C1=Cc1ccc(N2CCS(=O)(=O)CC2)s1] |
| 89 | 3-(difluoromethyl)-4-((5-(1- oxidothiomorpholino)thiophen-2- yl)methylene)isoxazol-5(4H)-one | 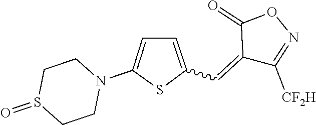 [SMILES: O=C1ON=C(C(F)F)C1=Cc1ccc(N2CCS(=O)CC2)s1] |
| 90 | 4-((5-(4-oxopiperidin-1- yl)thiophen-2-yl)methylene)-3- (perfluoroethyl)isoxazol-5(4H)-one | 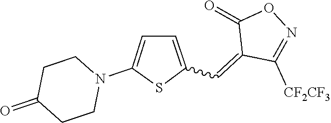 |
| 91 | 4-((5-(4-hydroxypiperidin-1- yl)thiophen-2-yl)methylene)-3- (perfluoroethyl)isoxazol-5(4H)-one | 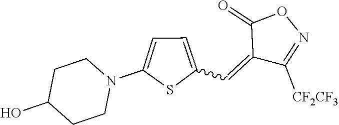 |
| 92 | 4-((5-(4-(2,2,2- trifluoroacetyl)piperazin-1- yl)thiophen-2-yl)methylene)-3- (trifluoromethyl)isoxazol-5(4H)-one | 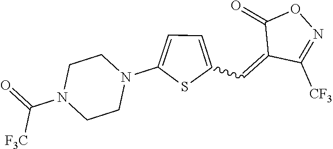 [SMILES: O=C1ON=C(C(F)(F)F)C1=Cc1ccc(N2CCN(C(=O)C(F)(F)F)CC2)s1] |
| 93 | 4-((5-(4-benzoylpiperazin-1- yl)thiophen-2-yl)methylene)-3- (trifluoromethyl)isoxazol-5(4H)-one | 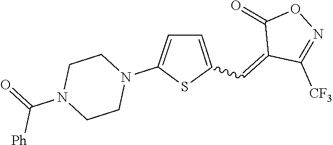 [SMILES: O=C1ON=C(C(F)(F)F)C1=Cc1ccc(N2CCN(C(=O)c3ccccc3)CC2)s1] |
| 94 | 4-((5-(diethylamino)thiophen-2- yl)methylene)-3- (perfluoroethyl)isoxazol-5(4H)-one | 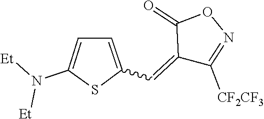 |
| 95 | 4-((5-(diethylamino)thiophen-2- yl)methylene)-3-methylisoxazol- 5(4H)-one | 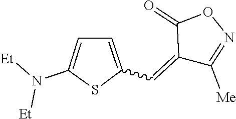 [SMILES: CCN(CC)c1ccc(C=C2C(=O)ON=C2C)s1] |
| 96 | 3-methyl-4-((5-(pyrrolidin-1- yl)thiophen-2- yl)methylene)isoxazol-5(4H)-one | 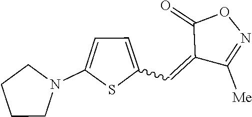 [SMILES: CC1=NOC(=O)C1=Cc1ccc(N2CCCC2)s1] |
| 97 | 1-(5-((3-methyl-5-oxoisoxazol- 4(5H)-ylidene)methyl)thiophen-2- yl)piperidin-4-yl phenylcarbamate | 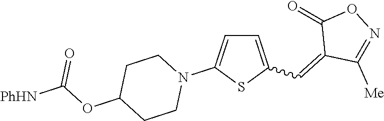 |
| 98 | 4-((5-(bis(3,3,3- trifluoropropyl)amino)thiophen-2- yl)methylene)-3- (trifluoromethyl)isoxazol-5(4H)-one | 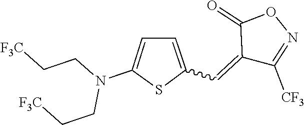 [SMILES: O=C1ON=C(C(F)(F)F)C1=Cc1ccc(N(CCC(F)(F)F)CCC(F)(F)F)s1] |
| 99 | 3-(tert-butyl)-4-((5- morpholinothiophen-2- yl)methylene)isoxazol-5(4H)-one | 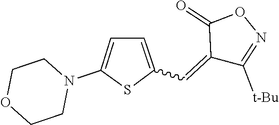 [SMILES: CC(C)(C)C1=NOC(=O)C1=Cc1ccc(N2CCOCC2)s1] |
| 100 | 2-(methyl(5-((5-oxo-3- (trifluoromethyl)isoxazol-4(5H)- ylidene)methyl)thiophen-2- yl)amino)ethyl phenylcarbamate | 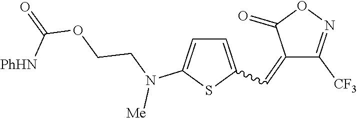 |
| 101 | 4-((5-(bis(2- methoxyethyl)amino)thiophen-2- yl)methylene)-3- (perfluoroethyl)isoxazol-5(4H)-one | 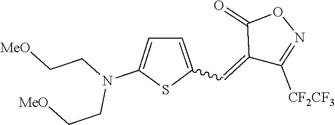 |
| 102 | 4-((5-(3-oxopyrrolidin-1- yl)thiophen-2-yl)methylene)-3- (trifluoromethyl)isoxazol-5(4H)-one | 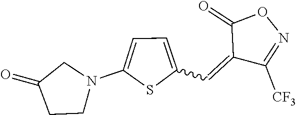 [SMILES: O=C1CCN(c2ccc(C=C3C(=O)ON=C3C(F)(F)F)s2)C1] |
| 103 | 4-((5-(3-oxoazetidin-1-yl)thiophen- 2-yl)methylene)-3- (trifluoromethyl)isoxazol-5(4H)-one | 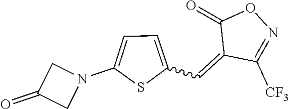 [SMILES: O=C1CN(c2ccc(C=C3C(=O)ON=C3C(F)(F)F)s2)C1] |
| 104 | 1-(5-((5-oxo-3- (trifluoromethyl)isoxazol-4(5H)- ylidene)methyl)thiophen-2- yl)azetidin-3-yl phenylcarbamate | 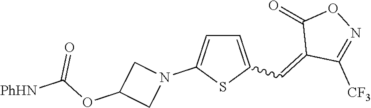 |
| 105 | 4-((5-(3,3-difluoroazetidin-1- yl)thiophen-2-yl)methylene)-3- (trifluoromethyl)isoxazol-5(4H)-one | 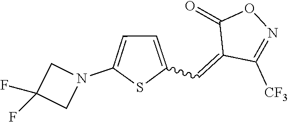 [SMILES: O=C1ON=C(C(F)(F)F)C1=Cc1ccc(N2CC(F)(F)C2)s1] |
| 106 | 4-((5-((2- hydroxyethyl)(methyl)amino)thio- phen-2-yl)methylene)-3- (trifluoromethyl)isoxazol-5(4H)-one | 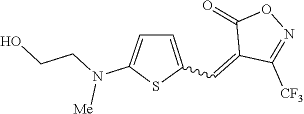 [SMILES: CN(CCO)c1ccc(C=C2C(=O)ON=C2C(F)(F)F)s1] |
| 107 | 4-((5-((2- hydroxyethyl)(methyl)amino)thio- phen-2-yl)methylene)-3- (trifluoromethyl)isoxazol-5(4H)-one | 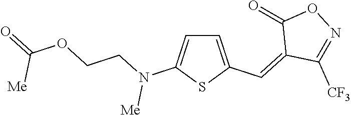 [SMILES: CC(=O)OCCN(C)c1ccc(/C=C2\C(=O)ON=C2C(F)(F)F)s1] |
| 108 | 2-(methyl(5-((5-oxo-3- (trifluoromethyl)isoxazol-4(5H)- ylidene)methyl)thiophen-2- yl)amino)ethyl phenylcarbamate | 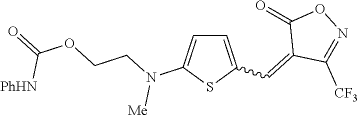 |
| 109 | 4-((5-(bis(2- hydroxyethyl)amino)thiophen-2- yl)methylene)-3- (trifluoromethyl)isoxazol-5(4H)-one | 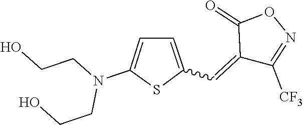 [SMILES: O=C1ON=C(C(F)(F)F)C1=Cc1ccc(N(CCO)CCO)s1] |
| 110 | 1-(5-((5-oxo-3- (trifluoromethyl)isoxazol-4(5H)- ylidene)methyl)thiophen-2- yl)piperidin-4-yl phenylcarbamate | 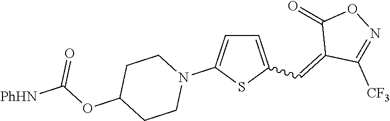 |
| 111 | 4-((5-(1,1- dioxidothiomorpholino)thiophen-2- yl)methylene)-3- (trifluoromethyl)isoxazol-5(4H)-one | 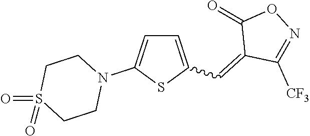 [SMILES: O=C1ON=C(C(F)(F)F)C1=Cc1ccc(N2CCS(=O)(=O)CC2)s1] |
| 112 | 4-((5-(1- oxidothiomorpholino)thiophen-2- yl)methylene)-3- (trifluoromethyl)isoxazol-5(4H)-one | 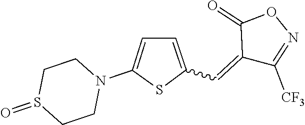 [SMILES: O=C1ON=C(C(F)(F)F)C1=Cc1ccc(N2CCS(=O)CC2)s1] |
| 113 | 4-((5-(4,4-difluoropiperidin-1- yl)thiophen-2-yl)methylene)-3- (trifluoromethyl)isoxazol-5(4H)-one | 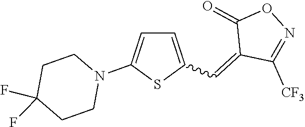 [SMILES: O=C1ON=C(C(F)(F)F)C1=Cc1ccc(N2CCC(F)(F)CC2)s1] |
| 114 | 3-(trifluoromethyl)-4-((5-(4- (trifluoromethyl)piperidin-1- yl)thiophen-2- yl)methylene)isoxazol-5(4H)-one | 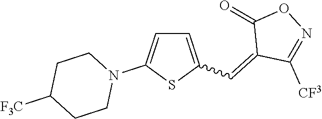 [SMILES: O=C1ON=C(C(F)(F)F)C1=Cc1ccc(N2CCC(C(F)(F)F)CC2)s1] |
| 115 | 1-(5-((5-oxo-3- (trifluoromethyl)isoxazol-4(5H)- ylidene)methyl)thiophen-2- yl)piperidine-4-carbonitrile | 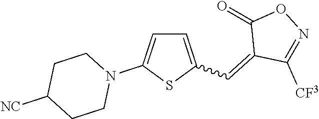 [SMILES: N#CC1CCN(c2ccc(C=C3C(=O)ON=C3C(F)(F)F)s2)CC1] |
| 116 | ethyl 1-(5-((5-oxo-3- (trifluoromethyl)isoxazol-4(5H)- ylidene)methyl)thiophen-2- yl)piperidine-4-carboxylate | 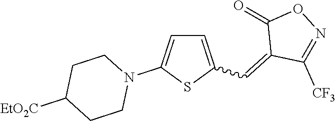 [SMILES: CCOC(=O)C1CCN(c2ccc(C=C3C(=O)ON=C3C(F)(F)F)s2)CC1] |
| 117 | ethyl 1-(5-((5-oxo-3- (trifluoromethyl)isoxazol-4(5H)- ylidene)methyl)thiophen-2- yl)piperidine-3-carboxylate | 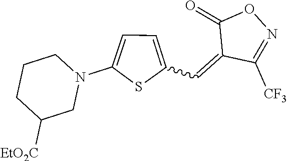 [SMILES: CCOC(=O)C1CCCN(c2ccc(C=C3C(=O)ON=C3C(F)(F)F)s2)C1] |
| 118 | 1-(5-((5-oxo-3- (trifluoromethyl)isoxazol-4(5H)- ylidene)methyl)thiophen-2- yl)piperidine-3-carbonitrile | 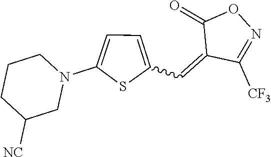 [SMILES: N#CC1CCCN(c2ccc(C=C3C(=O)ON=C3C(F)(F)F)s2)C1] |
| 119 | 4-((5-(3-oxopyrrolidin-1- yl)thiophen-2-yl)methylene)-3- (trifluoromethyl)isoxazol-5(4H)-one | 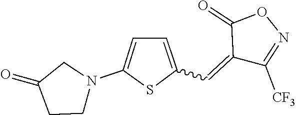 [SMILES: O=C1CCN(c2ccc(C=C3C(=O)ON=C3C(F)(F)F)s2)C1] |
| 120 | 4-((5-(3,3-difluoropyrrolidin-1- yl)thiophen-2-yl)methylene)-3- (trifluoromethyl)isoxazol-5(4H)-one | 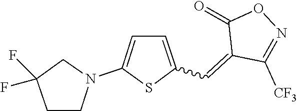 [SMILES: O=C1ON=C(C(F)(F)F)C1=Cc1ccc(N2CCC(F)(F)C2)s1] |
| 121 | 1-(5-((5-oxo-3- (trifluoromethyl)isoxazol-4(5H)- ylidene)methyl)thiophen-2- yl)pyrrolidine-3-carbonitrile | 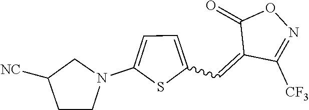 [SMILES: N#CC1CCN(c2ccc(C=C3C(=O)ON=C3C(F)(F)F)s2)C1] |
| 122 | 3-(trifluoromethyl)-4-((5-(3- (trifluoromethyl)pyrrolidin-1- yl)thiophen-2- yl)methylene)isoxazol-5(4H)-one | 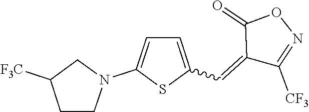 [SMILES: O=C1ON=C(C(F)(F)F)C1=Cc1ccc(N2CCC(C(F)(F)F)C2)s1] |
| 123 | ethyl 1-(5-((5-oxo-3- (trifluoromethyl)isoxazol-4(5H)- ylidene)methyl)thiophen-2- yl)pyrrolidine-3-carboxylate | 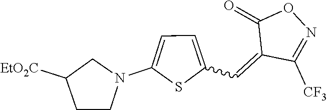 [SMILES: CCOC(=O)C1CCN(c2ccc(C=C3C(=O)ON=C3C(F)(F)F)s2)C1] |
| 124 | diethyl (3R,4S)-1-(5-((5-oxo-3- (trifluoromethyl)isoxazol-4(5H)- ylidene)methyl)thiophen-2- yl)pyrrolidine-3,4-dicarboxylate | 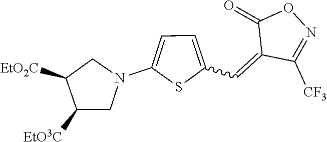 |
| 125 | diethyl (3R,4R)-1-(5-((5-oxo-3- (trifluoromethyl)isoxazol-4(5H)- ylidene)methyl)thiophen-2- yl)pyrrolidine-3,4-dicarboxylate | 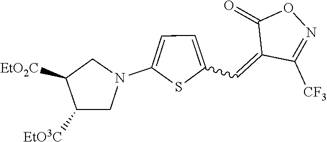 |
| 126 | (3R,4S)-1-(5-((5-oxo-3- (trifluoromethyl)isoxazol-4(5H)- ylidene)methyl)thiophen-2- yl)pyrrolidine-3,4-dicarbonitrile | 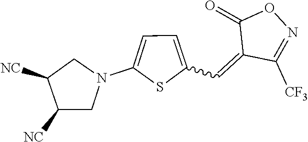 [SMILES: N#C[C@@H]1CN(c2ccc(C=C3C(=O)ON=C3C(F)(F)F)s2)C[C@@H]1C#N] |
| 127 | (3R,4R)-1-(5-((5-oxo-3- (trifluoromethyl)isoxazol-4(5H)- ylidene)methyl)thiophen-2- yl)pyrrolidine-3,4-dicarbonitrile | 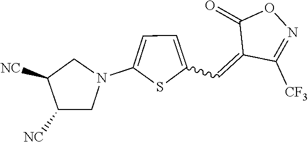 [SMILES: N#C[C@H]1CN(c2ccc(C=C3C(=O)ON=C3C(F)(F)F)s2)C[C@@H]1C#N] |
| 128 | ethyl (5-((5-oxo-3- (trifluoromethyl)isoxazol-4(5H)- ylidene)methyl)thiophen-2- yl)prolinate | 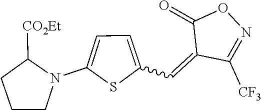 [SMILES: CCOC(=O)C1CCCN1c1ccc(C=C2C(=O)ON=C2C(F)(F)F)s1] |
| 129 | 3-(perfluoroethyl)-4-((5-(pyrrolidin- 1-yl)thiophen-2- yl)methylene)isoxazol-5(4H)-one | 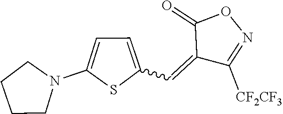 |
| 130 | 3-(difluoromethyl)-4-((5- (pyrrolidin-1-yl)thiophen-2- yl)methylene)isoxazol-5(4H)-one | 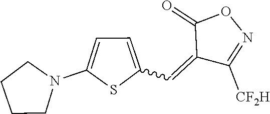 [SMILES: O=C1ON=C(C(F)F)C1=Cc1ccc(N2CCCC2)s1] |
| 131 | 4-((5-morpholinothiophen-2- yl)methylene)-3- (perfluoroethyl)isoxazol-5(4H)-one | 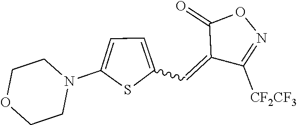 |
| 132 | 3-(difluoromethyl)-4-((5- morpholinothiophen-2- yl)methylene)isoxazol-5(4H)-one | 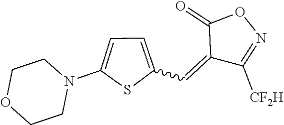 [SMILES: O=C1ON=C(C(F)F)C1=Cc1ccc(N2CCOCC2)s1] |
| 133 | 4-((5-(dimethylamino)-3- methylthiophen-2-yl)methylene)-3- (perfluoroethyl)isoxazol-5(4H)-one | 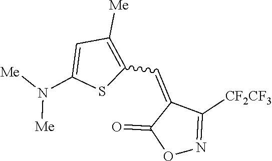 |
| 134 | 3-(difluoromethyl)-4-((5- (dimethylamino)-3-methylthiophen- 2-yl)methylene)isoxazol-5(4H)-one | 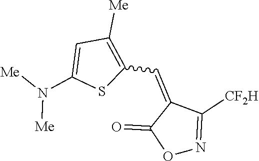 [SMILES: Cc1cc(N(C)C)sc1C=C1C(=O)ON=C1C(F)F] |
| 135 | 3-(methyl(5-((5-oxo-3- (perfluoroethyl)isoxazol-4(5H)- ylidene)methyl)thiophen-2- yl)amino)propanenitrile | 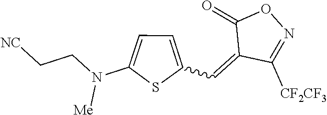 |
| 136 | 3-((5-((3-(difluoromethyl)-5- oxoisoxazol-4(5H)- ylidene)methyl)thiophen-2- yl)(methyl)amino)propanenitrile | 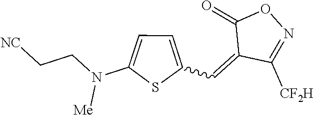 [SMILES: CN(CCC#N)c1ccc(C=C2C(=O)ON=C2C(F)F)s1] |
| 137 | diethyl 2,2′-((5-((5-oxo-3- (perfluoroethyl)isoxazol-4(5H)- ylidene)methyl)thiophen-2- yl)azanediyl)diacetate | 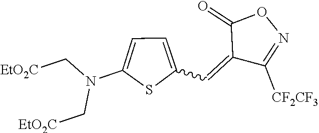 |
| 138 | diethyl 2,2′-((5-((3- (difluoromethyl)-5-oxoisoxazol- 4(5H)-ylidene)methyl)thiophen-2- yl)azanediyl)diacetate | 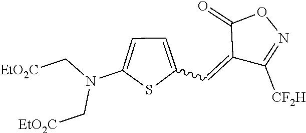 [SMILES: CCOC(=O)CN(CC(=O)OCC)c1ccc(C=C2C(=O)ON=C2C(F)F)s1] |
| 139 | 1-(5-((5-oxo-3- (perfluoroethyl)isoxazol-4(5H)- ylidene)methyl)thiophen-2- yl)piperidin-4-yl phenylcarbamate | 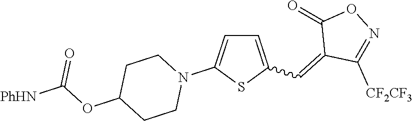 |
| 1409 | 4-((5-(4-acetylpiperazin-1- yl)thiophen-2-yl)methylene)-3- (perfluoroethyl)isoxazol-5(4H)-one | 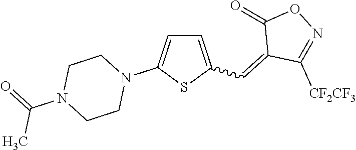 |
| 141 | 4-((5-(4-(methylsulfonyl)piperazin- 1-yl)thiophen-2-yl)methylene)-3- (perfluoroethyl)isoxazol-5(4H)-one | 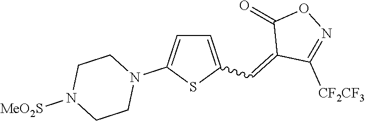 |
| 142 | 3-(trifluoromethyl)-4-((5-(4- ((trifluoromethyl)sulfonyl)piperazin- 1-yl)thiophen-2- yl)methylene)isoxazol-5(4H)-one | 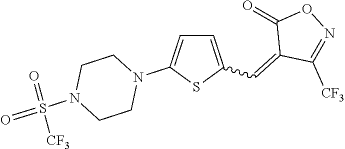 [SMILES: O=C1ON=C(C(F)(F)F)C1=Cc1ccc(N2CCN(S(=O)(=O)C(F)(F)F)CC2)s1] |
| 143 | 4-((5-(4-tosylpiperazin-1- yl)thiophen-2-yl)methylene)-3- (trifluoromethyl)isoxazol-5(4H)-one | 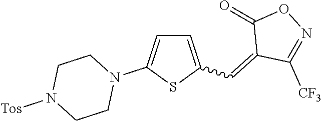 |
| 144 | 4-((5-(4-benzoylpiperazin-1- yl)thiophen-2-yl)methylene)-3- phenylisoxazol-5(4H)-one | 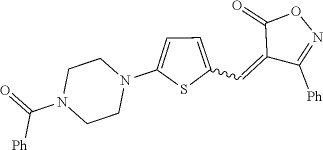 [SMILES: O=C1ON=C(c2ccccc2)C1=Cc1ccc(N2CCN(C(=O)c3ccccc3)CC2)s1] |
| 145 | 4-((5-(diethylamino)thiophen-2- yl)methylene)-3- (difluoromethyl)isoxazol-5(4H)-one | 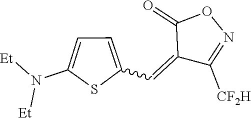 [SMILES: CCN(CC)c1ccc(C=C2C(=O)ON=C2C(F)F)s1] |
| 146 | 1-(5-((5-oxo-3- (trifluoromethyl)isoxazol-4(5H)- ylidene)methyl)thiophen-2- yl)piperidin-4-yl phenylcarbamate | 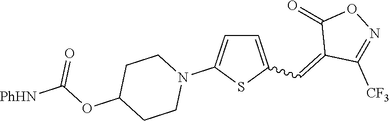 |
| 147 | 3-methyl-4-((5- morpholinothiophen-2- yl)methylene)isoxazol-5(4H)-one | 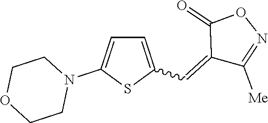 [SMILES: CC1=NOC(=O)C1=Cc1ccc(N2CCOCC2)s1] |
| 148 | benzyl 4-(5-((3-methyl-5- oxoisoxazol-4(5H)- ylidene)methyl)thiophen-2- yl)piperazine-1-carboxylate | 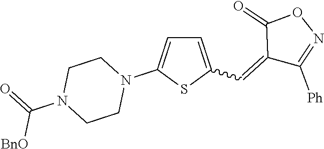 [SMILES: O=C1ON=C(c2ccccc2)C1=Cc1ccc(N2CCN(C(=O)OCc3ccccc3)CC2)s1] |
| 149 | 4-((5-(methyl(3,3,3- trifluoropropyl)amino)thiophen-2- yl)methylene)-3- (trifluoromethyl)isoxazol-5(4H)-one | 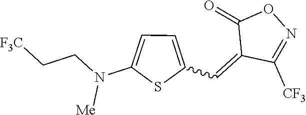 [SMILES: CN(CCC(F)(F)F)c1ccc(C=C2C(=O)ON=C2C(F)(F)F)s1] |
| 150 | 3-(tert-butyl)-4-((5-(4- hydroxypiperidin-1-yl)thiophen-2- yl)methylene)isoxazol-5(4H)-one | 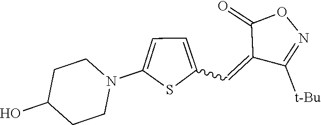 [SMILES: CC(C)(C)C1=NOC(=O)C1=Cc1ccc(N2CCC(O)CC2)s1] |
| 151 | 4-((5-(4-methoxypiperidin-1- yl)thiophen-2-yl)methylene)-3- (trifluoromethyl)isoxazol-5(4H)-one | 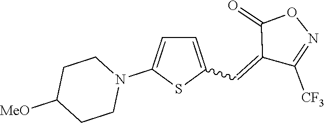 [SMILES: COC1CCN(c2ccc(C=C3C(=O)ON=C3C(F)(F)F)s2)CC1] |
| 152 | 4-((5-(4-methoxypiperidin-1- yl)thiophen-2-yl)methylene)-3- phenylisoxazol-5(4H)-one | 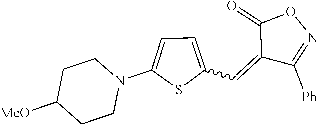 [SMILES: COC1CCN(c2ccc(C=C3C(=O)ON=C3c3ccccc3)s2)CC1] |
| 153 | 1-(5-((5-oxo-3- (trifluoromethyl)isoxazol-4(5H)- ylidene)methyl)thiophen-2- yl)azetidine-3-carbonitrile | 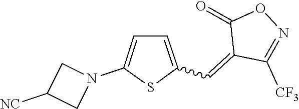 [SMILES: N#CC1CN(c2ccc(C=C3C(=O)ON=C3C(F)(F)F)s2)C1] |
| 154 | 4-((5-(3-hydroxyazetidin-1- yl)thiophen-2-yl)methylene)-3- (trifluoromethyl)isoxazol-5(4H)-one | 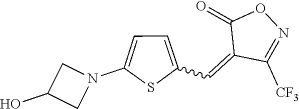 [SMILES: O=C1ON=C(C(F)(F)F)C1=Cc1ccc(N2CC(O)C2)s1] |
| 155 | ethyl 1-(5-((5-oxo-3- (trifluoromethyl)isoxazol-4(5H)- ylidene)methyl)thiophen-2- yl)azetidine-3-carboxylate | 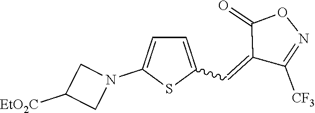 [SMILES: CCOC(=O)C1CN(c2ccc(C=C3C(=O)ON=C3C(F)(F)F)s2)C1] |
| 156 | 4-((5-(3-methoxyazetidin-1- yl)thiophen-2-yl)methylene)-3- (trifluoromethyl)isoxazol-5(4H)-one | 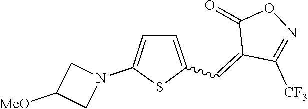 [SMILES: COC1CN(c2ccc(C=C3C(=O)ON=C3C(F)(F)F)s2)C1] |
| 157 | 3-(chlorodifluoromethyl)-4-((5- (dimethylamino)thiophen-2- yl)methylene)isoxazol-5(4H)-one | 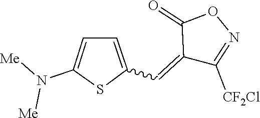 |
| 158 | 3-(dichlorofluoromethyl)-4-((5- (dimethylamino)thiophen-2- yl)methylene)isoxazol-5(4H)-one | 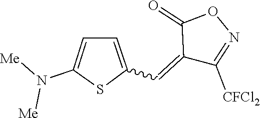 |

In another aspect, the present disclosure also encompasses intermediates suitable for preparing the compounds according to Formula I, such as aminothiophenes according to Formula II:

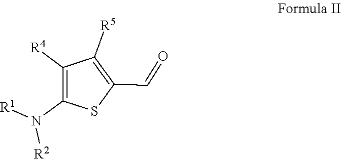

wherein
R¹ and R² can each, independently, be C₁-C₆ alkyl, C₁-C₆ alkylene-OH, alkylene C(O)OR⁶, alkylene nitrile, C₁-C₆ haloalkyl, SO₂-haloalkyl, C₁-C₆ alkylene-O-alkyl, or C₃-C₆ cycloalkyl; or R¹ and R² are taken together with the nitrogen to which they are bound to form an optionally substituted 4- to 7-membered heterocycle;
R⁴ and R⁵ can each, independently, be H, D, halo, C₁-C₆ alkyl, C₁-C₆ alkoxy, C₁-C₆ alkenyl, C₁-C₆ alkynyl, C₁-C₆ cycloalkyl, or C₆-C₁₀ aryl; or R⁴ and R⁵ can be taken together with the ring to which they are bound form an optionally substituted C₆-C₁₀ cycloalkyl optionally containing 1-3 heteroatoms, wherein the heteroatom can be O or N; and
R⁶ can be C₁-C₆ alkyl.

In another aspect, the present disclosure also encompasses intermediates suitable for preparing the compounds according to Formula I, such as 3-substituted-5-isoxazolones according to Formula III:

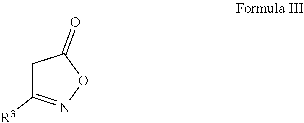

wherein
R³ can be C₁-C₆alkyl, C₁-C₆ haloalkyl, C₁-C₆ alkenyl, C₁-C₆ alkynyl, C₁-C₆ alkoxy, SO₂-alkyl, SO₂-haloalkyl, SO₂-aryl, C₁-C₆ cycloalkyl, C₁-C₆ alkylene-O-alkyl, nitrile, C(O)OR⁶, optionally substituted C₆-C₁₀ aryl, or optionally substituted C₃-C₉ heteroaryl; and
R⁶ can be C₁-C₆ alkyl.

### Methods of Making the Chroma Enhancing Dyes

The present disclosure also provides methods of making the dyes described herein. In certain aspects, the method comprises reacting a compound of Formula II with a compound of Formula III to provide a compound of Formula I, such as in Scheme 1, below.

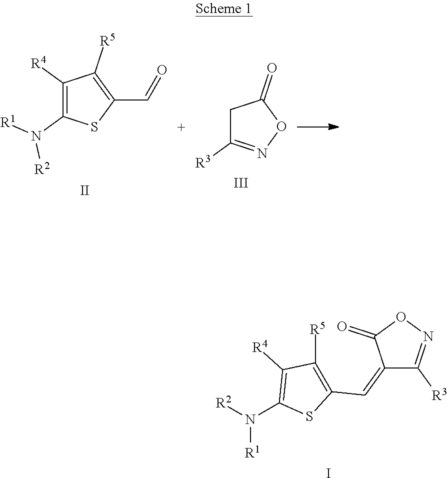

In some aspects, the compounds of Formula I can be prepared by a Knoevenagal-type condensation between a compound of Formula II and a compound of Formula III (Scheme 1).

In some aspects of the reaction, R¹ and R² can each, independently, be C₁-C₆ alkyl, C₁-C₆ alkylene-OH, alkylene C(O)OR⁶, alkylene nitrile, C₁-C₆ haloalkyl, SO₂-haloalkyl, C₁-C₆ alkylene-O-alkyl, or C₃-C₆ cycloalkyl; or R¹ and R² are taken together with the nitrogen to which they are bound to form an optionally substituted 4- to 7-membered heterocycle;
R³ can be C₁-C₆alkyl, C₁-C₆ haloalkyl, C₁-C₆ alkenyl, C₁-C₆ alkynyl, C₁-C₆ alkoxy, SO₂-alkyl, SO₂-haloalkyl, SO₂-aryl, C₁-C₆ cycloalkyl, C₁-C₆ alkylene-O-alkyl, nitrile, C(O)OR⁶, optionally substituted C₆-C₁₀ aryl, or optionally substituted C₃-C₉ heteroaryl;
R⁴ and R⁵ can each, independently, be H, D, halo, C₁-C₆ alkyl, C₁-C₆ alkoxy, C₁-C₆ alkenyl, C₁-C₆ alkynyl, C₁-C₆ cycloalkyl, or C₆-C₁₀ aryl; or R⁴ and R⁵ can be taken together with the ring to which they are bound form an optionally substituted C₆-C₁₀ cycloalkyl optionally containing 1-3 heteroatoms; and
R⁶ can be C₁-C₆ alkyl, provided that when R¹ and R² are both ethyl, R³ is not phenyl.

In some aspects, R¹ and R² can each, independently, be methyl, ethyl, n-propyl, n-butyl, (CH₂)ₘCO₂R⁶, (CH₂)ₘCN, (CH₂)ₘCF₃, or C₃-C₆ cycloalkyl, or R¹ and R² can be taken together with the nitrogen to which they are bound to form a 4- to 7-membered heterocycle, wherein the 4- to 7-membered heterocycle is

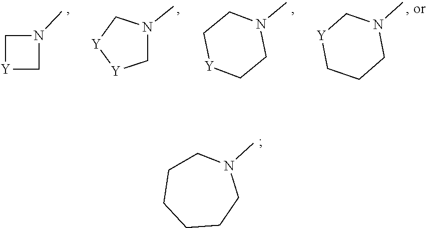

R³ can be methyl, ethyl, t-butyl, methoxy, ethoxy, CF₃, CF₂H, CH₂F, CF₂CF₃, CH₂Cl, CCl₃, phenyl, pyridin-4-yl, pyridin-3-yl, pyridine-2-yl, or 4-methoxyphenyl;
R⁴ and R⁵ can each independently be selected from H, D, methyl, ethyl, i-propyl, t-butyl, n-butyl, halo, methoxy, ethoxy, cycloalkyl, or phenyl;
R⁶ can be C₁-C₆ alkyl;
each m can independently be 1, 2, 3, or 4; and
Y in each instance can, independently, be CH₂, O, S, SO, SO₂, CF₂, NR⁷, N—C(O)R⁷, N—SO₂—R⁷, N—C(O)OR⁷, N—C(O)N(R⁷)₂, CO, C(H)OR⁷, C(H)CN, C(H)OC(O)NHR⁷, C(H)CO₂R⁷, C(H)CF₃, C(H)-alkyl, or C(H)-aryl; and
R⁷ can be H, alkyl, haloalkyl, optionally substituted phenyl, benzyl or phenylalkyl, provided that when R¹ and R² are both ethyl, R³ is not phenyl.

In still further aspects, R¹ and R² can each, independently, be methyl, ethyl, or n-butyl;
R³ can be methyl, ethyl, CF₃, CF₂H, CF₂CF₃, or phenyl; and
R⁴ and R⁵ can each, independently, be H, methyl, halo, or phenyl, provided that when R¹ and R² are both ethyl, R³ is not phenyl.

In another aspect, the present disclosure relates to a process of preparing compounds of Formula II (Scheme 2):

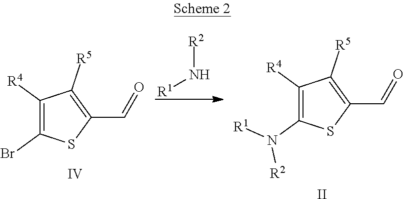

In some aspects, the compounds of Formula II can be prepared by an SNAr reaction of an amine according to the formula NHR₁R², with a substituted 5-bromothiophene-2-carbaldehyde of Formula IV.

In some aspects of the reaction for preparing compounds of Formula II, R¹ and R² can each, independently, be C₁-C₆ alkyl, C₁-C₆ alkylene-OH, alkylene C(O)OR⁶, alkylene nitrile, C₁-C₆ haloalkyl, SO₂-haloalkyl, C₁-C₆ alkylene-O-alkyl, or C₃-C₆ cycloalkyl, or R¹ and R² can be taken together with the nitrogen to which they are bound to form an optionally substituted 4- to 7-membered heterocycle;
R⁴ and R⁵ can each, independently, be H, D, halo, C₁-C₆ alkyl, C₁-C₆ alkoxy, C₁-C₆ alkenyl, C₁-C₆ alkynyl, C₁-C₆ cycloalkyl, or C₆-C₁₀ aryl; or R⁴ and R⁵ can be taken together with the ring to which they are bound form an optionally substituted C₆-C₁₀ cycloalkyl optionally containing 1-3 heteroatoms; and
R⁶ can be C₁-C₆ alkyl.

In certain aspects of the reaction for preparing compounds of Formula II, R¹ and R² can each, independently, be methyl, ethyl, n-propyl, n-butyl, (CH₂)ₘCO₂R⁶, (CH₂)ₘCN, (CH₂)ₘCF₃, or C₃-C₆ cycloalkyl, or R¹ and R² can be taken together with the nitrogen to which they are bound to form a 4- to 6-membered heterocycle, wherein the 4- to 7-membered heterocycle is

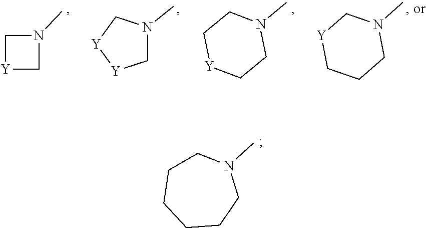

R⁴ and R⁵ can each independently be selected from H, D, methyl, ethyl, i-propyl, t-butyl, n-butyl, halo, methoxy, ethoxy, cycloalkyl, or phenyl; or R⁴ and R⁵ can be taken together with the ring to which they are bound form an optionally substituted C₆-C₁₀ cycloalkyl optionally containing 1-3 heteroatoms;
R⁶ can be C₁-C₆ alkyl;
each m can, independently, be 1, 2, 3, or 4;
Y in each instance can, independently, be CH₂, O, S, SO, SO₂, CF₂, NR⁷, N—C(O)R⁷, N—SO₂—R⁷, N—C(O)OR⁷, N—C(O)N(R⁷)₂, CO, C(H)OH, C(H)OR⁷, C(H)CN, C(H)OC(O)NHR⁷, C(H)CO₂R⁷, C(H)CF₃, C(H)-alkyl, or C(H)-aryl; and
R⁷ can be H, alkyl, haloalkyl, optionally substituted phenyl, benzyl or phenylalkyl.

In still further aspects of the reaction, R¹ and R² can each, independently, be methyl, ethyl, or n-butyl and
R⁴ and R⁵ can each, independently, be H, methyl, halo, or phenyl.

In another aspect, the present disclosure relates to a process of preparing compounds of Formula III (Scheme 3):

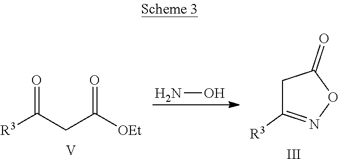

In some aspects of the reaction, the compounds of Formula III can be prepared by reacting hydroxylamine with β-ketoesters of Formula V.

In some aspects of the reaction, R³ can be C₁-C₆alkyl, C₁-C₆ haloalkyl, C₁-C₆ alkenyl, C₁-C₆ alkynyl, C₁-C₆ alkoxy, SO₂-alkyl, SO₂-haloalkyl, SO₂-aryl, C₁-C₆ cycloalkyl, C₁-C₆ alkylene-O-alkyl, nitrile, C(O)OR⁶, optionally substituted C₆-C₁₀ aryl, or optionally substituted C₃-C₉ heteroaryl; and
R⁶ can be C₁-C₆ alkyl.

In still further aspects, R³ can be methyl, ethyl, t-butyl, methoxy, ethoxy, CF₃, CF₂H, CH₂F, CF₂CF₃, CH₂Cl, CCl₃, phenyl, pyridin-4-yl, pyridin-3-yl, pyridine-2-yl, or 4-methoxyphenyl.

In still further aspects of the reaction, R³ can be methyl, ethyl, CF₃, CF₂H, CF₂CF₃, or phenyl.

Eyewear with Chroma Enhancement

The present disclosure further provides eyewear comprising the chroma-enhancing dyes of Formula I. In typical aspects, the eyewear can comprise a lens comprising the dye of Formula I. The compound of Formula I can be incorporated into the lens in any acceptable manner that results in chroma enhancement. In certain aspects, the lens can comprise an optical filter comprising the dye of Formula I. In some aspects, the optical filter comprising the dye of Formula I can be incorporated into a lens having any desired curvature, including, for example, cylindrical, plano, spherical or toroidal. In some aspects, the lens can include one or more functional components, such as, for example, layers, coatings, or laminates. Examples of functional components include color enhancement filters, chroma enhancement filters, laser attenuation filter, electrochromic filters, photoelectrochromic filters, polarizing filter, variable attenuation filters, anti-reflection coatings, interference stacks, hard coatings, flash mirrors, anti-static coatings, anti-fog coatings, blue cut dye layers, deep red absorbing dye layers, and broadly absorbing dye layers, other functional layers, or a combination of functional layers. In some aspects, the lens can include a lens body and the optical filter comprising the dye of Formula I.

In some aspects, a lens including a lens body and an optical filter comprising the dye of Formula I within and/or outside of the lens body can be configured to attenuate visible light in one or more spectral bands. In aspects in which the optical filter comprising the dye of Formula I is within the lens body, the optical filter comprising the dye of Formula I can constitute the lens body, or the optical filter comprising the dye of Formula I and additional components can constitute the lens body. In some aspects, the optical filter comprising the dye of Formula I can be configured to substantially increase the colorfulness, clarity, and/or vividness of a scene. The optical filter comprising the dye of Formula I can be particularly suited for use with eyewear and can allow the wearer of the eyewear to view a scene with improved dynamic visual acuity.

The vividness of interpreted colors is correlated with an attribute known as the chroma value of a color. The chroma value is one of the attributes or coordinates of the CIE L*C*h color space. Together with attributes known as hue and lightness, the chroma can be used to define colors that are perceivable in human vision. It has been determined that visual acuity can be positively correlated with the chroma values of colors in a scene. In other words, the visual acuity of an observer can be greater when viewing a scene with high chroma value colors than when viewing the same scene with lower chroma value colors as described in U.S. Pat. No. 10,073,282 which is incorporated herein in its entirety. In some aspects, the optical filter comprising the dye of Formula I is optimized for specific applications, including, for example, driving and various sports, e.g., track, golf, and cycling as described in U.S. Pat. Nos. 10,073,282 and 9,134,547 which is incorporated by reference herein in its entirety.

In some aspects, the optical filter comprising the dye of Formula I is at least partially incorporated into the lens body. For example, and in some aspects, the lens body can be impregnated with, loaded with, or otherwise comprise one or more dyes of Formula I, such any of the compounds provide in Table 1 or Table 2, and in particular aspects 4-((5-(dimethylamino)thiophen-2-yl)methylene)-3-phenylisoxazol-5(4H)-one; 4-((5-(dimethylamino)thiophen-2-yl)methylene)-3-(trifluoromethyl)isoxazol-5(4H)-one; 4-((5-(diethylamino)thiophen-2-yl)methylene)-3-(trifluoromethyl)isoxazol-5(4H)-one; and 4-((5-(piperidin-1-yl)thiophen-2-yl)methylene)-3-(trifluoromethyl)isoxazol-5(4H)-one. Each of the one or more chroma-enhancing dyes of Formula I can be selected to produce an absorbance peak in one of the plurality of spectral bands. In some aspects, the optical filter comprising the dye of Formula I is at least partially incorporated into a lens coating disposed over the lens body. In other aspects, the optical filter comprising the dye of Formula I can further comprise an additional dye, such as Exciton ABS 407 dye, Exciton ABS 473 dye, Exciton ABS 515 dye, Exciton ABS 574 dye, Exciton ABS 647 dye, Exciton ABS 659 dye, or other organic dyes supplied by other vendors, such as Epolin Inc, Crysta-lyn, QCR Solutions Corp., HW Sands Corp, Yamada Chemical Co., and Gentex Corp.

The optical filter comprising the dye of Formula I can be configured to remove the outer portions of a broad visual stimulus to make colors appear more vivid as perceived in human vision. The outer portions of a broad visual stimulus refer to wavelengths that, when substantially, nearly completely, or completely attenuated, decrease the bandwidth of the stimulus such that the vividness of the perceived color is increased. Thus, an optical filter system for eyewear can be configured to substantially increase the colorfulness, clarity, and/or vividness of a scene. Such an optical filter system for eyewear can allow the wearer to view the scene in high definition color (HD color). In some aspects, portions of a visual stimulus that are not substantially attenuated include at least the wavelengths for which cone photoreceptor cells in the human eye have the greatest sensitivity. In certain aspects, the bandwidth of the color stimulus when the optical filter is applied includes at least the wavelengths for which the cone photoreceptor cells have the greatest sensitivity. In some aspects, a person wearing a lens incorporating an optical filter system disclosed herein can perceive a substantial increase in the clarity of a scene. The increase in perceived clarity can result, for example, from increased contrast, increased chroma, or a combination of factors.

In certain aspects, the optical filter comprising the dye of Formula I can be configured to enhance the chroma profile of a scene when the scene is viewed through a lens that incorporates the optical filter system. The optical filter system can be configured to increase or decrease chroma in one or more chroma enhancement windows (CEW) in order to achieve any desired effect. The chroma-enhancing optical filter system can be configured to preferentially transmit or attenuate light in any desired chroma enhancement windows. Any suitable process can be used to determine the desired chroma enhancement windows. For example, the colors predominantly reflected or emitted in a selected environment can be measured, and a filter can be adapted to provide chroma enhancement in one or more spectral regions corresponding to the colors that are predominantly reflected or emitted. Additionally, enhancing chroma in at least one chroma enhancement window can increase colorfulness, clarity, and/or vividness of a scene viewed through the lens. Aspects of eyewear including one or more optical filters with chroma enhancement material that increase chroma in at least one chroma enhancement window are described in U.S. Patent Publication No. 2011/0255051 which is incorporated by reference herein in its entirety.

The ability to identify and discern moving objects is generally called “Dynamic Visual Acuity.” Generally, dynamic visual acuity can decrease in the darkened state of various aspects of lenses. An increase in chroma (or chroma enhancement) in the spectral region of the moving object can improve the dynamic visual acuity because increases in chroma can be generally associated with higher color contrast. Furthermore, the emphasis and de-emphasis of specific colors can further improve dynamic visual acuity.

In some aspects, the optical filter comprising the dye of Formula I can be included in eyewear that can transition between a darkened state and a faded state to improve dynamic visual acuity in the darkened state. In some aspects of the optical filter comprising the dye of Formula I can be configured to be controllable by a user, by a sensor, and/or a logic to transition between a darkened state and a faded state. In certain aspects, the optical filter comprising the dye of Formula I can provide additional benefits such as increasing the colorfulness, clarity, and/or vividness of a scene viewed through the sunglasses in the darkened state and/or faded state.

In some aspects, a substantial chroma enhancement can be provided by an optical filter comprising the dye of Formula I that is configured to increase chroma in one or more chroma enhancement windows (CEWs) over a neutral filter without any significant decrease in chroma compared to a neutral filter within the one or more CEWs. In some aspects, a substantial chroma enhancement can also be provided by an optical filter comprising the dye of Formula I configured to increase chroma in one or more CEWs over a neutral filter without any significant decrease in chroma compared to a neutral filter within a particular spectral range, such as, for example, between about 420 nm and about 650 nm as described in U.S. Patent Publication No. 2022/0107511 which is incorporated by reference herein in its entirety.

In some aspects, the optical filter comprising the one or more compounds of Formula I selectively blocks visible light within a spectral band of wavelengths ranging from about 450 nm to about 510 nm; about 450 nm to about 495 nm; or about 475 nm to about 510 nm.

In some aspects, the optical filter comprising the one or more compounds of Formula I further comprises one or more additional chroma-enhancing dyes.

In another aspect, is at least partially incorporated into a lens coating, an adhering layer, a polarizing layer, a photochromic layer, an anti-reflection layer, other functional layer, or is at least partially incorporated into a combination of coatings and layers.

In some aspects, the optical filter comprising the one or more compounds of Formula I further comprises a polymeric material.

In some aspects, the one or more compounds of Formula I are at least partially incorporated into the polymeric material.

In some aspects, the one or more compounds of Formula I are
4-((5-(dimethylamino)thiophen-2-yl)methylene)-3-phenylisoxazol-5(4H)-one;
4-((5-(dimethylamino)thiophen-2-yl)methylene)-3-(perfluoroethyl)isoxazol-5(4H)-one;
3-(difluoromethyl)-4-((5-(dimethylamino)thiophen-2-yl)methylene)isoxazol-5(4H)-one;
4-((5-(dimethylamino)thiophen-2-yl)methylene)-3-methylisoxazol-5(4H)-one;
4-((5-(dimethylamino)-3-methylthiophen-2-yl)methylene)-3-(trifluoromethyl)isoxazol-5(4H)-one;
4-((5-(dimethylamino)thiophen-2-yl)methylene)-3-(trifluoromethyl)isoxazol-5(4H)-one;
4-((5-(dimethylamino)-3-methylthiophen-2-yl)methylene)-3-phenylisoxazol-5(4H)-one;
4-((5-(piperidin-1-yl)thiophen-2-yl)methylene)-3-(trifluoromethyl)isoxazol-5(4H)-one;
3-phenyl-4-((5-(piperidin-1-yl)thiophen-2-yl)methylene)isoxazol-5(4H)-one;
3-(perfluoroethyl)-4-((5-(piperidin-1-yl)thiophen-2-yl)methylene)isoxazol-5(4H)-one;
3-(difluoromethyl)-4-((5-(piperidin-1-yl)thiophen-2-yl)methylene)isoxazol-5(4H)-one;
4-((5-(pyrrolidin-1-yl)thiophen-2-yl)methylene)-3-(trifluoromethyl)isoxazol-5(4H)-one;
3-phenyl-4-((5-(pyrrolidin-1-yl)thiophen-2-yl)methylene)isoxazol-5(4H)-one;
4-((5-morpholinothiophen-2-yl)methylene)-3-(trifluoromethyl)isoxazol-5(4H)-one;
4-((5-morpholinothiophen-2-yl)methylene)-3-phenylisoxazol-5(4H)-one;
3-phenyl-4-((5-(piperazin-1-yl)thiophen-2-yl)methylene)isoxazol-5(4H)-one;
4-((5-(4-(methylsulfonyl)piperazin-1-yl)thiophen-2-yl)methylene)-3-phenylisoxazol-5(4H)-one;
4-((5-thiomorpholinothiophen-2-yl)methylene)-3-(trifluoromethyl)isoxazol-5(4H)-one;
3-phenyl-4-((5-thiomorpholinothiophen-2-yl)methylene)isoxazol-5(4H)-one;
4-((5-(1,1-dioxidothiomorpholino)thiophen-2-yl)methylene)-3-phenylisoxazol-5(4H)-one;
4-((5-(1-oxidothiomorpholino)thiophen-2-yl)methylene)-3-phenylisoxazol-5(4H)-one;
3-methyl-4-((5-(piperidin-1-yl)thiophen-2-yl)methylene)isoxazol-5(4H)-one;
4-((5-(1,1-dioxidothiomorpholino)thiophen-2-yl)methylene)-3-(perfluoroethyl)isoxazol-5(4H)-one; and
4-((5-(1-oxidothiomorpholino)thiophen-2-yl)methylene)-3-(perfluoroethyl)isoxazol-5(4H)-one.

In some aspects, the one or more compounds of Formula I is (E)-4-((5-(diethylamino)thiophen-2-yl)methylene)-3-(trifluoromethyl)isoxazol-5(4H)-one, (Z)-4-((5-(diethylamino)thiophen-2-yl)methylene)-3-(trifluoromethyl)isoxazol-5(4H)-one, or a combination thereof.

In some aspects, the one or more compounds of Formula I is (E)-4-((5-(dimethylamino)thiophen-2-yl)methylene)-3-methylisoxazol-5(4H)-one, (Z)-4-((5-(dimethylamino)thiophen-2-yl)methylene)-3-methylisoxazol-5(4H)-one, or a combination thereof.

In some aspects, the one or more compounds of Formula I is (E)-4-((5-morpholinothiophen-2-yl)methylene)-3-(trifluoromethyl)isoxazol-5(4H)-one, (Z)-4-((5-morpholinothiophen-2-yl)methylene)-3-(trifluoromethyl)isoxazol-5(4H)-one, or a combination thereof.

In some aspects, the one or more compounds of Formula I is (E)-4-((5-(dimethylamino)thiophen-2-yl)methylene)-3-(trifluoromethyl)isoxazol-5(4H)-one, (Z)-4-((5-(dimethylamino)thiophen-2-yl)methylene)-3-(trifluoromethyl)isoxazol-5(4H)-one, or a combination thereof.

In some aspects, the optical filter comprising the dye of Formula I has a peak optical density (OD) of between about 0.05 and about 6.00 OD; about 0.10 and about 4.00 OD; about 0.10 and about 3.00 OD; about 0.10 and about 2.00 OD; about 0.10 and about 2.00 OD; about 0.10 and about 1.00 OD; about 0.15 and about 0.45 OD; or about 0.25 and about 0.55 OD; about 1.00 and about 2.00 OD; about 1.70 and about 1.80 OD.

In some aspects, the optical filter comprising the dye of Formula I is incorporated into eyewear. In some aspects, the optical filter comprising the dye of Formula I can be particularly suited for use with eyewear and can allow the wearer of the eyewear to view a scene with improved dynamic visual acuity.

FIG. 1 illustrates a perspective view of an eyewear 100 including a pair of lenses 102a, 102b that comprise the optical filter comprising one or more chroma-enhancing dyes including one or more compounds of Formula I configured to provide chroma enhancement, according to some aspects. The eyewear can be of any type, including general-purpose eyewear, special-purpose eyewear, sunglasses, eyeshields, eyewear incorporated into headworn support (such as visors for helmets), visors, driving glasses, sporting glasses, goggles, vision-correcting eyewear, prescription and non-prescription eyeglasses, color vision deficiency eyewear, indoor eyewear, outdoor eyewear, contrast-enhancing eyewear, chroma-enhancing eyewear, color-enhancing eyewear, color-altering eyewear, gaming eyewear, eyewear designed for another purpose, or eyewear designed for a combination of purposes. In some aspects, lenses and frames of many other shapes and configurations may be used for eyewear 100. For example, eyewear 100 can have a single, unitary lens, such as in a goggle or visor. It should be noted that eyewear 100 shown in FIG. 1 is not drawn to scale but is drawn to more easily illustrate certain aspects of eyewear 100.

In some aspects, a lens can comprise a lens body and an optical filter comprising one or more compounds of Formula I.

In another embodiment, the optical filter can be the lens body. In another embodiment, the optical filter can be partially incorporated into the lens body.

In some aspects, the one of more compounds of Formula I can be present in the lens in a range of about 0.0002% to about 6%; about 0.002% to about 6%; about 0.02% to about 6%; about 0.2% to about 6%; about 0.0002% to about 5%; about 0.2% to about 4%; about 0.2% to about 3%; or about 0.2% to about 2% by weight.

In some aspects, the one of more compounds of Formula I and one or more additional chroma-enhancing dyes can be present in the lens in a range of about 0.0002% to about 6%; about 0.002% to about 6%; about 0.02% to about 6%; about 0.2% to about 6%; about 0.0002% to about 5%; about 0.2% to about 4%; about 0.2% to about 3%; or about 0.2% to about 2% by weight.

Weight percent is calculated based on [i] the total weight of the chroma-enhancing dyes incorporated into the lens including any compounds of Formula I and any additional-chroma enhancing dyes relative to [ii] the total weight of the lens.

The pair of lenses 102a, 102b can be implemented in any headworn support (i.e., a headworn article that can support one or more lenses in the wearer's field of view). For example, other headworn supports can include, but are not limited to, helmets, face masks, balaclavas, and breaching shields. The lenses 102a and 102b can be corrective lenses or non-corrective lenses and can be made of any of a variety of optical materials including glass and/or plastics, such as, for example, acrylics or polycarbonates. The lenses can have various shapes. For example, the lenses 102a, 102b can be flat, have 1 axis of curvature, 2 axes of curvature, or more than 2 axes of curvature, the lenses 102a, 102b can be cylindrical, parabolic, spherical, flat, or elliptical, or any other shape such as a meniscus or catenoid. When worn, the lenses 102a, 102b can extend across the wearer's normal straight ahead line of sight, and can extend substantially across the wearer's peripheral zones of vision. As used herein, the wearer's normal line of sight shall refer to a line projecting straight ahead of the wearer's eye, with substantially no angular deviation in either the vertical or horizontal planes. In some aspects, the lenses 102a, 102b extend across a portion of the wearer's normal straight ahead line of sight.

The outside surface of lenses 102a or 102b can conform to a shape having a smooth, continuous surface having a constant horizontal radius (sphere or cylinder) or progressive curve (ellipse, toroid or ovoid) or other aspheric shape in either the horizontal or vertical planes. The geometric shape of other aspects can be generally cylindrical, having curvature in one axis and no curvature in a second axis. The lenses 102a, 102b can have a curvature in one or more dimensions. For example, the lenses 102a, 102b can be curved along a horizontal axis. As another example, lenses 102a, 102b can be characterized in a horizontal plane by a generally arcuate shape, extending from a medial edge throughout at least a portion of the wearer's range of vision to a lateral edge. In some aspects, the lenses 102a, 102b are substantially linear (not curved) along a vertical axis. In some aspects, the lenses 102a, 102b have a first radius of curvature in one region, a second radius of curvature in a second region, and transition sites disposed on either side of the first and second regions. The transition sites can be a coincidence point along the lenses 102a, 102b where the radius of curvature of the lenses 102a, 102b transitions from the first to the second radius of curvature, and vice versa. In some aspects, lenses 102a, 102b can have a third radius of curvature in a parallel direction, a perpendicular direction, or some other direction. In some aspects, the lenses 102a, 102b can lie on a common circle. The right and left lenses in a high-wrap eyeglass can be canted such that the medial edge of each lens will fall outside of the common circle and the lateral edges will fall inside of the common circle. Providing curvature in the lenses 102a, 102b can result in various advantageous optical qualities for the wearer, including reducing the prismatic shift of light rays passing through the lenses 102a, 102b, and providing an optical correction.

A variety of lens configurations in both horizontal and vertical planes are possible. Thus, for example, either the outer or the inner or both surfaces of the lens 102a or 102b of some aspects can generally conform to a spherical shape or to a right circular cylinder. Alternatively either the outer or the inner or both surfaces of the lens can conform to a frustoconical shape, a toroid, an elliptic cylinder, an ellipsoid, an ellipsoid of revolution, other asphere or any of a number of other three dimensional shapes. Regardless of the particular vertical or horizontal curvature of one surface, however, the other surface can be chosen such as to minimize one or more of power, prism, and astigmatism of the lens in the mounted and as-worn orientation.

The lenses 102a, 102b can be linear (not curved) along a vertical plane (e.g., cylindrical or frustoconical lens geometry). In some aspects, the lenses 102a, 102b can be aligned substantially parallel with the vertical axis such that the line of sight is substantially normal to the anterior surface and the posterior surface of the lenses 102a, 102b. In some aspects, the lenses 102a, 102b are angled downward such that a line normal to the lens is offset from the straight ahead normal line of sight by an angle Φ. The angle (of offset can be greater than about 0° and/or less than about 30°, or greater than about 700 and/or less than about 20°, or about 15°, although other angles Φ outside of these ranges can also be used. Various cylindrically shaped lenses can be used. The anterior surface and/or the posterior surface of the lenses 102a, 102b can conform to the surface of a right circular cylinder such that the radius of curvature along the horizontal axis is substantially uniform. An elliptical cylinder can be used to provide lenses that have non-uniform curvature in the horizontal direction. For example, a lens can be more curved near its lateral edge than its medial edge. In some aspects, an oblique (non-right) cylinder can be used, for example, to provide a lens that is angled in the vertical direction.

In some aspects, the eyewear 100 incorporates canted lenses 102a, 102b mounted in a position rotated laterally relative to conventional centrally oriented dual lens mountings. A canted lens can be conceived as having an orientation, relative to the wearer's head, which would be achieved by starting with conventional dual lens eyewear having centrally oriented lenses and bending the frame inwardly at the temples to wrap around the side of the head. When the eyewear 100 is worn, a lateral edge of the lens wraps significantly around and comes in close proximity to the wearer's temple to provide significant lateral eye coverage.

A degree of wrap may be desirable for aesthetic styling reasons, for lateral protection of the eyes from flying debris, or for interception of peripheral light. Wrap can be attained by utilizing lenses of tight horizontal curvature (high base), such as cylindrical or spherical lenses, and/or by mounting each lens in a position which is canted laterally and rearwardly relative to centrally oriented dual lenses. Similarly, a high degree of rake or vertical tilting may be desirable for aesthetic reasons and for intercepting light, wind, dust or other debris from below the wearer's eyes. In general, “rake” will be understood to describe the condition of a lens, in the as-worn orientation, for which the normal line of sight strikes a vertical tangent to the lens 102a or 102b at a non-perpendicular angle.

The lenses 102a, 102b can be provided with anterior and posterior surfaces and a thickness therebetween, which can be variable along the horizontal direction, vertical direction, or combination of directions. In some aspects, the lenses 102a, 102b can have a varying thickness along the horizontal or vertical axis, or along some other direction. In some aspects, the thickness of the lenses 102a, 102b tapers smoothly, though not necessarily linearly, from a maximum thickness proximate a medial edge to a relatively lesser thickness at a lateral edge. The lenses 102a, 102b can have a tapering thickness along the horizontal axis and can be decentered for optical correction. In some aspects, the lenses 102a, 102b can have a thickness configured to provide an optical correction. For example, the thickness of the lenses 102a, 102b can taper from a thickest point at a central point of the lenses 102a, 102b approaching lateral segments of the lenses 102a, 102b. In some aspects, the average thickness of the lenses 102a, 102b in the lateral segments can be less than the average thickness of the lenses 102a, 102b in the central zone. In some aspects, the thickness of the lenses 102a, 102b in at least one point in the central zone can be greater than the thickness of the lenses 102a, 102b at any point within at least one of the lateral segments.

In some aspects, the lenses 102a, 102b can be finished, as opposed to semi-finished, with the lenses 102a, 102b being contoured to modify the focal power. In some aspects, the lenses 102a, 102b can be semi-finished so that the lenses 102a, 102b can be capable of being machined, at some time following manufacture, to modify their focal power. In some aspects, the lenses 102a, 102b can have optical power and can be prescription lenses configured to correct for near-sighted or far-sighted vision. The lenses 102a, 102b can have cylindrical characteristics to correct for astigmatism.

The eyewear 100 can include a mounting frame 104 configured to support the lenses 102a, 102b. The mounting frame 104 can include orbitals that partially or completely surround the lenses 102a, 102b. Referring to FIG. 1, it should be noted that the particular mounting frame 104 is not essential to the aspect disclosed herein. The frame 104 can be of varying configurations and designs, and the illustrated aspect shown in FIG. 1 is provided as examples only. As illustrated, the frame 104 can include a top frame portion and a pair of ear stems 106a, 106b that are connected to opposing ends of the top frame portion. Further, the lenses 102a, 102b can be mounted to the frame 104 with an upper edge of the lens 102a or 102b extending along or within a lens groove and being secured to the frame 104. For example, the upper edge of the lens 102a or 102b can be formed in a pattern, such as a jagged or non-linear edge, and apertures or other shapes around which the frame 104 can be injection molded or fastened in order to secure the lens 102a or 102b to the frame 104. Further, the lenses 102a, 102b can be removably attachable to the frame 104 by means of a slot with inter-fitting projections or other attachment structure formed in the lenses 102a, 102b and/or the frame 104.

It is also contemplated that the lenses 102a, 102b can be secured along a lower edge of the frame 104. Various other configurations can also be utilized. Such configurations can include the direct attachment of the ear stems 106a, 106b to the lenses 102a, 102b without any frame, or other configurations that can reduce the overall weight, size, or profile of the eyeglasses. In addition, various materials can be utilized in the manufacture of the frame 104, such as metals, composites, or relatively rigid, molded thermoplastic materials which are well known in the art, and which can be transparent or available in a variety of colors. Indeed, the mounting frame 104 can be fabricated according to various configurations and designs as desired. In some aspects, the frame 104 is configured to retain a unitary lens that is placed in front of both eyes when the eyewear is worn. Eyewear (e.g., goggles) can also be provided that include a unitary lens that is placed in front of both eyes when the eyewear is worn. The unitary lens having features similar to the lenses 102a, 102b can be implemented in other types of headworn supports such as, but not limited to, helmets, face masks, balaclavas, and breaching shields.

In some aspects, the ear stems 106a, 106b can be pivotably attached to the frame 104. In some aspects, the ear stems 106a, 106b attach directly to the lenses 102a, 102b. The ear stems 106a, 106b can be configured to support the eyewear 100 when worn by a user. For example, the ear stems 106a, 106b can be configured to rest on the ears of the user. In some aspects, the eyewear 100 includes a flexible band used to secure the eyewear 100 in front of the user's eyes in place of ear stems 106a, 106b.

In some aspects of the lenses 102a and 102b that includes the optical filter comprising one or more chroma-enhancing dyes including one or more compounds of Formula I, can comprise a lens body 108 and a lens component 110, as illustrated in FIG. 1A. The lens body 108 can have an inner surface facing the eye and an outer surface opposite the inner surface. The inner surface and/or the outer surface of the lens body 108 can be curved (e.g., convex or concave). In some aspects, the inner and/or the outer surface of the lens body 108 can be planar. The lens component 110 can be substantially permanently affixed to the lens body 108, or the lens component 110 can be configured to be separable from the lens body 108. The lens component 110 can be attached to the inner or outer surface of the lens body 108. In some aspects, the lens component 110 can be configured to be removable such that a user, manufacturer, or retailer can apply, remove, or change the lens component 110 after manufacture of the eyewear 100. In this way, a variety of functional elements can be introduced into the eyewear 100 increasing the possible utility of the eyewear 100 because the eyewear can be altered to provide functionality suitable for different occasions. In some aspects, the lens component 110 includes a laminate, a coating, a flexible material, an inflexible material, an insert molded component, a chip, a gel layer, a liquid layer, an air gap, a filter, or any combination of components.

The lens body 108 that incorporates or is the optical filter comprising one or more chroma-enhancing dyes including one or more compounds of Formula I can be formed of polymer, polycarbonate (or PC), allyl diglycol carbonate monomer (being sold under the brand name CR-39®), glass, polyamide, polyurethane, polyethylene, polyimide, polyethylene terephthalate (or PET), biaxially-oriented polyethylene terephthalate polyester film (or BoPET, with one such polyester film sold under the brand name MYLAR®), acrylic (polymethyl methacrylate or PMMA), a polymeric material, a co-polymer, a doped material, any other suitable material, or any combination of materials.

The lens body 108 can be rigid and other layers of the lens can conform to the shape of the lens body 108 such that the lens body 108 dictates the shape of the lens 102a or 102b. The lens body 108 can be symmetrical across a vertical axis of symmetry, symmetrical across a horizontal axis of symmetry, symmetrical across another axis, or asymmetrical. In some aspects, the front and back surfaces of the lens body 108 can conform to the surfaces of respective cylinders that have a common center point and different radii. In some aspects, the lens body can have a front and back surfaces that conform to the surfaces of respective cylinders that have center points offset from each other, such that the thickness of the lens body 108 tapers from a thicker central portion to thinner end portions. The surfaces of the lens body 108 can conform to other shapes, as discussed herein, such as a sphere, toroid, ellipsoid, asphere, plano, frustoconical, and the like. In some aspects, a thermoforming process, a molding process, a casting process, a lamination process, an extrusion process, an adhering process, and/or another suitable process can be used to attach the lens component 110 to the lens body 108 having a shape described herein.

The lens body 108 can be contoured during initial formation to have an optical magnification characteristic that modifies the focal power of the lens 102a or 102b. In some aspects, the lens body 108 can be machined after initial formation to modify the focal power of the lens 102a or 102b. The lens body 108 can provide a substantial amount of the optical power and magnification characteristics to the lens 102a or 102b. In some aspects, the lens body 108 provides the majority of the optical power and magnification characteristics. Apportioning the majority of optical power and magnification to the lens body 108 can permit selection of lens body 108 materials and lens body 108 formation techniques that provide improved lens 102a, 102b optical power and magnification characteristics, without adversely affecting selection of lens component 110 materials and formation techniques.

In some aspects, the lens body 108 can be injection molded, although other processes can be used to form the shape of the lens blank body, such as thermoforming, casting, or machining. In some aspects, the lens body 108 is injection molded and includes a relatively rigid and optically acceptable material such as polycarbonate. The curvature of the lens body 108 would thus be incorporated into a molded lens blank. A lens blank can include the desired curvature and taper in its as-molded condition. One or two or more lens bodies of the desired shape can then be cut from the optically appropriate portion of the lens blank as is understood in the art. In some aspects, the frame 104 is provided with a slot or other attachment structure that cooperates with the molded and cut shape of the lens body 108 and lens component 110 to minimize deviation from, and even improve retention of its intended shape. In some aspects, the lens body 108 can be stamped or cut from flat sheet stock and then bent into the curved configuration using a process such as thermoforming. This curved configuration can then be maintained by the use of a relatively rigid, curved frame 104, or by heating the curved sheet to retain its curved configuration.

The lens component 110 can be attached to the lens body 108, for example, through a thermally-cured adhesive layer, a UV-cured adhesive layer, electrostatic adhesion, pressure sensitive adhesives, or any combination of these. Examples of bonding technologies that can be suitable for attaching the lens component 110 to the lens body 108 include thermal welding, fusing, pressure sensitive adhesives, polyurethane adhesives, electrostatic attraction, thermoforming, other types of adhesives, materials curable by ultraviolet light, thermally curable materials, radiation-curable materials, other bonding methods, other bonding materials, and combinations of methods and/or materials. In some aspects, any technique suitable for affixing the lens component 110 to the lens body 108 can be used. Some aspects of a lens 102a or 102b includes a lens body 108 and a lens component 110 that are bonded together. In some aspects, the lens component 110 and the lens body 108 can be integrally connected to each other and can be adhesively bonded together.

The lens component 110 can include a single layer or multiple layers. The lens component 110 can have one or more layers in single or multiple layer form that can be coated with a hard coat or a primer. For example, the lens component 110 can be a single layer of polycarbonate, PET, polyethylene, acrylic, polyamide, polyurethane, polyimide, BoPET, another film material, or a combination of materials. As another example, the lens component can include multiple layers of film, where each film layer includes polycarbonate, PET, polyethylene, acrylic, polyamide, polyurethane, polyimide, BoPET, another film material, or a combination of materials.

Each of the lens component 110 and/or lens body 108 can include one or more layers that serve various functions within the lenses 102a, 102b. In some aspects, one or more layers in the lens component 110 and/or the lens body 108 can provide optical properties to the lenses 102a, 102b such as optical filtering, polarization, photochromism, electrochromism, photoelectrochromism and/or partial reflection of incoming visible light, chroma enhancement, color enhancement, color alteration, or any combination of these. In some aspects, one or more layers within the lens component 110 and/or the lens body 108 can provide mechanical protection to the lenses 102a, 102b or other layers within the lens component 110, reduce stresses within the lens component 110, or improve bonding or adhesion among the layers in the lens component 110 and/or between the lens component 110 and the lens body 108. In some aspects, the lens component 110 and/or the lens body 108 can include layers that provide additional functionality to the lenses 102a, 102b such as, for example, anti-reflection functionality, anti-static functionality, anti-fog functionality, scratch resistance, mechanical durability, hydrophobic functionality, reflective functionality, darkening functionality, aesthetic functionality including tinting, or any combination of these.

Accordingly some aspects of the lens body 108 and/or lens component 110 can include a polarizing layer configured to provide polarization, one or more adhesive layers, a photochromic layer, an electrochromic layer configured to provide electrochromism, a photoelectrochomic layer, reflective layer configured to provide a partial reflection of incoming visible light, a hard coat, a flash mirror, a liquid-containing layer, an antireflection layer, a mirror layer, an interference stack, chroma enhancing dyes, an index-matching layer, a scratch resistant coating, a hydrophobic coating, an absorption layer configured to provide a partial or complete absorption of infrared light, a color enhancement layer, a color alteration layer, an anti-static coating, an anti-fog functional layer, chroma enhancement dyes, color enhancement elements, a darkening functional layer, an aesthetic functional layer including tinting, laser attenuation filters, trichoic filters, violet edge filter, UV filter, IR filter, glass layers, hybrid glass-plastic layers, anti-reflective coatings, contrast enhancement elements, a liquid-containing layer, a gel containing layer, a refractive index matching layer, thermal insulation layer, electrical insulation layer, electrical conducting layer, neutral density filter, other lens elements, or a combination of lens components. In some aspects, the optical filter comprising the dye of Formula I is at least partially incorporated into a lens body and/or lens component or one or more functional layers. In some aspects, the optical filter comprising the dye of Formula I is at least partially incorporated into lens body 108 and/or lens component 110 or one or more functional layers (e.g. a lens coating, an adhering layer, a polarizing layer, a photochromic layer, an anti-reflection layer, or is at least partially incorporated into a combination of coatings and layers). In some aspects, the process of forming the lens body can include forming a chroma enhancement wafer, and forming a lens substrate over the chroma enhancement wafer. By way of example and not limitation, the process of forming a chroma enhancement wafer can include injection molding or casting a wafer that includes one or more wavelength filtering materials. By way of example and not limitation, the process of forming the lens body over the wavelength chroma enhancement wafer can include placing the chroma enhancement wafer in a mold cavity and molding an optically transparent material, such as resin, over one or more surfaces of the wavelength filtering wafer in the mold cavity. As a result, after the optically transparent material (e.g., resin) is cooled down and hardened, the lens body can conform to the wavelength filtering wafer. It is noted that the above described embodiments for forming the lens body are exemplary and not limiting. Various implementations for forming the lens body are described, for example, in U.S. Patent Publication. No. 2017/0075143, which is incorporated by reference herein in its entirety.

As an example, the lens component 110 can include one or more layers that can serve to thermally insulate the lens component 110 such that it can be used in high temperature molding processes without subjecting the certain functional layers to temperatures sufficient to significantly degrade their optical performance. In some aspects, the lens component 110 can serve as a thermally isolating element or vehicle that can incorporate functional elements that can be degraded if subjected to high temperature manufacturing processes. As such, the lens component 110 can be used to incorporate these types of functional elements into lenses that otherwise are formed and/or manufactured using high temperature processes. As another example, the lens component 110 can include a substrate with one or more functional coatings deposited thereon. The functional coatings can include elements that would be degraded or whose performance would be altered if subjected to high temperatures, such as certain chroma enhancement dyes. The lens component 110 could then be bonded to the lens body 108 using a UV-cured adhesive, thus thermally isolating the lens component 110 and the included functional layers from the high temperature processes associated with the manufacture of the lens body 108.

In certain aspects, lens 102a or 102b that includes the optical filter comprising one or more chroma-enhancing dyes including one or more compounds of Formula I can also incorporate addition functionality. The lens component 110 or the lens body 108 can include layers or elements that serve to tint the lens 102a, 102b. Tinting can be added to a lens element in different ways. In some aspects, color can be deposited on the lens element using a vapor or liquid source. The color can coat the lens element or it can penetrate into the element, and/or can be applied using a sublimation process. In some aspects, color can be added to a material used to make the lens element, such as adding powdered color or plastic pellets to material that is extruded, injection molded, or otherwise molded into a lens element. In some aspects where liquids are used, the color can be added by a dip process. In such aspects, a gradient tint or bi-gradient tint can be achieved through the dip process. In certain aspects, a liquid coloring technique can be used to tint one or more lens elements. For example, liquid dye can be added to the polymer during an injection molding process.

In some aspects, the lens 102 can comprise an optical filter system comprising an optical filter comprising one or more chroma-enhancing dyes including one or more compounds of Formula I with variable optical characteristics (also referred to as a variable optical filter) including a variable filter component 114 and an optical filter with fixed optical characteristics (also referred to as a static filter) including a static filter component 116, as illustrated in FIG. 1B and as described in U.S. Pat. No. 10,073,282.

In some aspects, the method of manufacturing a lens can include forming a lens having an optical filter comprising one or more chroma-enhancing dyes including one or more compounds of Formula I configured to attenuate visible light in a plurality of spectral bands. Each of the plurality of spectral bands can include an absorbance peak with a spectral bandwidth, a maximum absorbance, and an integrated absorptance peak area within the spectral bandwidth. An attenuation factor of the absorbance peak in each of the plurality of spectral bands can be greater than or equal to about 0.8 and less than 1. In some aspects, a lens can be formed by forming a lens body and forming a lens coating over the lens body. At least a portion of the optical filter can be incorporated into the lens body or the lens coating. In some aspects, the lens coating can include an interference coating. In some aspects, the lens can include a UV absorption layer or a layer that includes UV absorption outside of the optical filter layer. Such a layer can decrease bleaching of the optical filter. In addition, UV absorbing agents can be disposed in any lens component or combination of lens components. In some aspects, a lens body can be formed by a method including forming a plurality of lens body elements and coupling the lens body elements to one another using one or more adhering layers. A polarizing film can be disposed between two or more of the plurality of lens body elements. In some aspects, the polarizing film can be insert molded within the lens body. In some aspects, the lens can include one or more components that substantially absorb ultraviolet radiation, including near ultraviolet radiation. As described in U.S. Patent Publication No. 2022/0107511.

In certain aspects, the compound of Formula I can be added to a molten resin before the resin is injected into a mold cavity to form the lens body. By way of example and not limitation, the optically transparent material can include molten resin, polycarbonate (PC), allyl diglycol carbonate monomer (being sold under the brand name CR-39®), a resin layer (e.g., MR-8@), glass, polyamide, polyurethane, polyethylene, polyureas, polyamide (PA), polyethylene terephthalate (PET), biaxially-oriented polyethylene terephthalate polyester film (BoPET, with one such polyester film sold under the brand name MYLAR®), acrylic (polymethyl methacrylate or PMMA), triacetate cellulose (TAC), a polymeric material, a co-polymer, a doped material, any other suitable material, or any combination thereof as described in U.S. Patent Publication No. 2021/0157170 which is incorporated by reference herein in its entirety.

In some aspects, the process of forming the lens body can include forming a lens substrate, and forming a chroma enhancement layer over the lens substrate. By way of example and not limitation, a forming process of the lens substrate can include applying an injection molding process, a thermoforming process, a casting process, or a machining process on the optically transparent material described above, as described in U.S. Patent Publication No. 2021/0157170. Other examples of forming the lens body having chroma enhancement are disclosed in U.S. Patent Publication. No. 2017/0075143 (noted and incorporated by reference above) and U.S. Patent Publication. No. 2017/0102558, entitled “Eyewear with multiple functional layers,” which is also incorporated herein by reference in its entirety.

### EXAMPLES

The examples presented below is provided for the purpose of illustration only and the aspects described herein should in no way be construed as being limited to these examples. Rather, the aspects should be construed to encompass any and all variations which become evident as a result of the teaching provided herein.

### Example 1: General Procedures

### General Synthesis of Compounds of Formula II

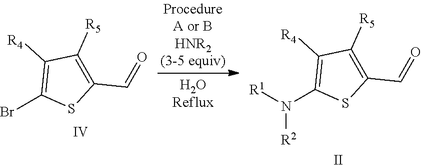

5-bromothiophene-2-carbaldehydes of Formula IV including 5-morpholinothiophene-2-carbaldehyde, 5-thiomorpholinothiophene-2-carbaldehyde, 5-(piperazin-1-yl)thiophene-2-carbaldehyde, tert-butyl 4-(5-formylthiophen-2-yl)piperazine-1-carboxylate, and 5-(4-hydroxypiperidin-1-yl)thiophene-2-carbaldehyde were purchased from commercial sources such as Enamine, Ambeed, or Sigma-Aldrich.

Additional aldehydes of Formula IV were prepared from commercially available starting materials using the compound-specific procedures described below in Examples 3-8.

Procedure A: A liquid solution of a 5-bromothiophene-2-carbaldehyde (IV) (1 equivalent) was combined with the desired amino nucleophile (3 to 5 equivalents) and H₂O (equal mass as amine) in a round bottom flask. The mixture was heated to reflux. The consumption of 5-bromothiophene-2-carbaldehyde was monitored by thin layer chromotagraphy (TLC). The reaction was cooled and diluted with H₂O. The product was extracted into EtOAc. The organic layer was dried with Na₂SO₄ and concentrated by rotary evaporation. The residue was diluted in CH₂Cl₂ and filtered through a short silica plug. The filtrate was concentrated. The product was precipitated from a mixture of EtOH/hexanes and isolated by vacuum filtration (if product is a solid) or concentrated by rotary evaporation (if product is an oil). Procedure B: A liquid solution of a 5-bromothiophene-2-carbaldehyde (IV) (1 equivalent) was combined with the desired amino nucleophile (3 to 5 equivalents) and H₂O (equal mass as amine) in a round bottom flask. Next, p-toluenesulfonic acid (0.05 equivalent) was added and the mixture was heated at reflux. The consumption of 5-bromothiophene-2-carbaldehyde was monitored by TLC. The reaction was cooled and diluted with H₂O. The product was extracted into EtOAc. The organic layer was dried with Na₂SO₄ and concentrated by rotary evaporation. The residue was diluted in CH₂Cl₂ and filtered through a short silica plug. The filtrate was concentrated. The product was precipitated from a mixture of EtOH/hexanes and isolated by vacuum filtration (if product is a solid) or concentrated by rotary evaporation (if product is an oil).

TABLE 3
Representative compounds of Formula II prepared using general procedures A or B.

| Compound | Chemical Name | Structure | Comments |
|---|---|---|---|
| 159 | 5-(dimethylamino)thiophene-2- carbaldehyde | 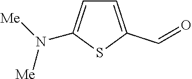 | Synthesized via Procedure A, 8.73 g (91% yield). ¹H NMR (CDCl₃): 9.49 (s, 1H), 7.48 |
| | | | (d, 1H), 5.94 (d, |
| | | | 1H), 3.10 (s, 6H). |
| | | | Melting point: |
| | | | 91° C. |
| 160 | 5-(diethylamino)thiophene-2- carbaldehyde | 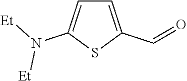 [SMILES: CCN(CC)c1ccc(C=O)s1] | Synthesized via Procedure A, 1.61 g (56% yield). ¹H NMR (CDCl₃): 9.46 (s, 1H), 7.46 |
| | | | (d, 1H), 5.92 (d, |
| | | | 1H), 3.42 (quart, |
| | | | 4H), 1.25 (t, 6H). |
| 161 | 5-(dibutylamino)thiophene-2- carbaldehyde | 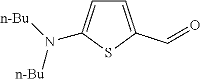 | Synthesized via Procedure A, 14.1 g (66% yield). ¹H NMR (CDCl₃: 9.46 (s, 1H, 7.44 (d, 1H), 5.90 (d, |
| | | | 1H), 3.33 (t, 4H), |
| | | | 1.64 (quint, 4H), |
| | | | 1.36 (quint, 4H), |
| | | | 0.97 (t, 6H). |
| 162 | 5-(piperidin-1-yl)thiophene-2- carbaldehyde | 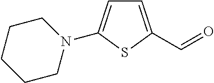 [SMILES: O=Cc1ccc(N2CCCCC2)s1] | Synthesized via Procedure B, 0.99 g (71% yield). ¹H NMR (CDCl₃): 9.51 (s, 1H), 7.48 (d, 1H), 6.07 (d, |
| | | | 1H), 3.35 (t, 4H), |
| | | | 1.62-1.75 (m, 6H). |
| 163 | 5-(dimethylamino)-3- methylthiophene-2- carbaldehyde | 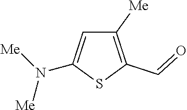 [SMILES: Cc1cc(N(C)C)sc1C=O] | Synthesized via Procedure A using 5-bromo-3- methylthiophene- 2-carbaldehyde, 0.72 g (23% yield). ¹H NMR |
| | | | (CDCl₃): 9.62 (s, |
| | | | 1H), 5.73 (s, 1H), |
| | | | 3.06 (s, 6H), 2.41 |
| | | | (s, 3H). |
| 164 | 5-(pyrrolidin-1-yl)thiophene-2- carbaldehyde | 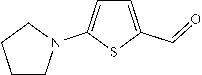 [SMILES: O=Cc1ccc(N2CCCC2)s1] | Synthesized via Procedure B with DMSO as co- solvent, 0.40 g (43% yield). ¹H |
| | | | NMR (CDCl₃): |
| | | | 9.47 (s, 1H), 7.48 |
| | | | (d, 1H), 5.85 (d, |
| | | | 1H), 3.35-3.41 (m, |
| | | | 4H), 2.06-2.12 (m, |
| | | | 4H). |
| 165 | 5-(bis(2- methoxyethyl)amino)thiophene- 2-carbaldehyde | 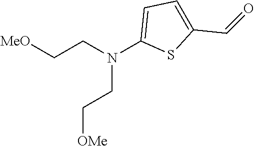 [SMILES: COCCN(CCOC)c1ccc(C=O)s1] | Synthesized via Procedure B, 0.24 g (25% yield). MS (APCI+): 244.0 m/z. |
| 166 | 5-(azepan-1-yl)thiophene-2- carbaldehyde | 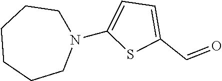 [SMILES: O=Cc1ccc(N2CCCCCC2)s1] | Synthesized via Procedure B, 1.51 g (quant. yield). 1H NMR (CDCl3): 9.45 (s, |
| | | | 1H), 7.45 (d, 1H), |
| | | | 5.92 (d, 1H), 3.48 |
| | | | (m, 4H), 1.80 (m, |
| | | | 4H), 1.51 (m, 4H). |
| 167 | 5-(azetidin-1-yl)thiophene-2- carbaldehyde | 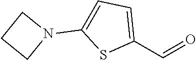 [SMILES: O=Cc1ccc(N2CCC2)s1] | Synthesized via Procedure B from 5- chlorothiophene- |
| | | | 2-carbaldehyde, |
| | | | 71 mg (29% |
| | | | yield). 1H NMR |
| | | | (CDCl3): 9.49 (s, |
| | | | 1H), 7.43 (d, 1H), |
| | | | 5.80 (d, 1H), 4.06 |
| | | | (t, 4H), 2.49 (m, |
| | | | 2H). |
| 168 | 5-(3-hydroxypyrrolidin-1- yl)thiophene-2-carbaldehyde | 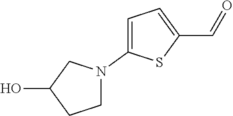 [SMILES: O=Cc1ccc(N2CCC(O)C2)s1] | Synthesized via Procedure B, 61 mg (6% yield, first crop). 1H NMR (CDCl3): 9.39 (s, 1H), 7.68 (d, 1H), 6.01 (d, 1H), 5.15 (d, 1H), 4.44 (m, |
| | | | 1H), 3.35-3.55 (m, |
| | | | 3H), 3.19 (d, 1H), |
| | | | 2.05-2.17 (m, 1H), |
| | | | 1.89-1.99 (m, 1H) |
| 169 | 7-(dimethylamino)-2,3- dihydrothieno[3,4- b][1,4]dioxine-5-carbaldehyde | 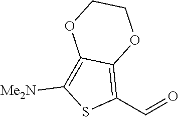 [SMILES: CN(C)c1sc(C=O)c2c1OCCO2] | Synthesized via Procedure B from 7-bromo-2,3- dihydrothieno[3,4- b][1,4] dioxine-5- carbaldehyde, 81 mg (63% yield). 1H NMR |
| | | | (CDCl3): 9.58 (s, |
| | | | 1H), 4.28-4.33 (m, |
| | | | 2H), 4.17-4.22 (m, |
| | | | 2H), 3.08 (s, 6H). |

### General Synthesis of Compounds of Formula III and IIIa

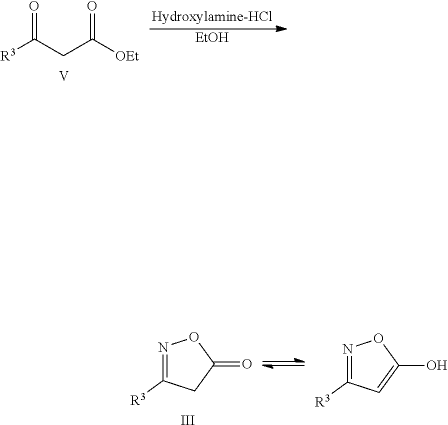

The compounds of Formula III can be prepared as described by the general procedure below or purchased from a commercial vendor. 3-phenylisoxazol-5(4H)-one and 3-(pyridin-4-yl)isoxazol-5(4H)-one were purchased from commercial vendors and used as received.

In a round-bottom flask, β-keto ester of Formula IV (1 equivalent), EtOH (1.3 M), and hydroxylamine hydrochloride (2.1 equivalent) were combined. The mixture was heated to reflux. The consumption of β-Keto ester was monitored by gas chromatography. Upon completion, the reaction was cooled to room temperature and diluted with CH₂Cl₂. The mixture was filtered to remove insoluble salts. The product mixture was transferred to a separatory funnel and extracted with CH₂O₂ and H₂. The pH was adjusted with NaOH (1.9 M aqueous solution) until pH was between 4 and 5. The organic layer was collected and dried with Na₂SO₄. The salts were filtered and the organic layer was concentrated by rotary evaporation to obtain product, i.e. the desired 3-substituted-isoxazol-5(4H)-one. Yields ranged from 85-95% with purity >9500 by GC analysis. The products were used without further purification. In some cases, typically in the absence of basic workup, uncyclized oxime intermediates IIIa were obtained and were used in the final step to provide compounds according to Formula I. In some cases, β-keto esters were dissolved in CHCl₃ and treated with hydroxylamine hydrochloride and sodium acetate to form isoxazolone III or oxime IIa intermediates, which were treated with an aminothiophene-substituted aldehyde and catalyst to yield final dyes without workup or isolation of the intermediates.

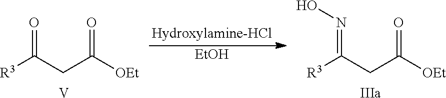

| Compound | Chemical Name | Structure | NMR Data |
|---|---|---|---|
| 170 | ethyl 4,4,4-trifluoro-3- (hydroxyimino)butanoate | 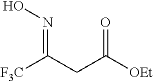 [SMILES: CCOC(=O)C/C(=N/O)C(F)(F)F] | ¹H NMR (CDCl₃): 9.13 (br s, 1H), 4.20 (quart, 2H), 3.52 (s, 2H), 1.27 (t, 3H) ¹⁹F NMR (CDCl₃): −69.97 |
| 171 | ethyl 4,4,5,5,5-pentafluoro-3- (hydroxyimino)pentanoate | 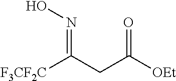 | ¹H NMR (CDCl₃): 9.97 (br s, 1H), 4.19 (quart, 2H), 3.50 (s, 2H), 1.25 (t, 3H) ¹⁹F NMR (CDCl₃): −82.20, −115.92 |
| 172 | ethyl 4,4-difluoro-3- (hydroxyimino)butanoate | 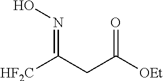 | ¹H NMR (CDCl₃): 8.76 (br s, 1H), 6.14 (t, 1H), 4.19 (quart, 2H), 3.47 (s, 2), 1.26 (t, 3H) ¹⁹F NMR (CDCl₃): −119.47, −119.62 |
| 173 | 3-methylisoxazol-5(4H)-one | 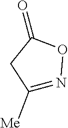 | ¹H NMR (CDCl₃): 3.40 (s, 2H), 2.16 (s, 3H) Reaction performed with NaOAc as base. |
| 174 | 3-(tert-butyl)isoxazol-5(4H)-one | 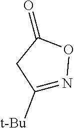 | ¹H NMR (CDCl₃): 3.41 (s, 2H), 1.25 (s, 9H) Reaction performed with NaOAc as base. |
| 175 | 3-(4- (trifluoromethyl)phenyl)isoxazol- 5(4H)-one | 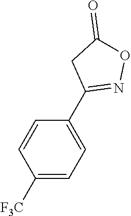 [SMILES: O=C1CC(c2ccc(C(F)(F)F)cc2)=NO1] | ¹H NMR (CDCl₃): 7.82 (d, 2H), 7.75 (d, 2H), 3.84 (s, 2H) ¹⁹F NMR (CDCl₃): −63.17 Reaction performed with NaOAc as base. |
| 176 | 3-(4-methoxyphenyl)isoxazol- 5(4H)-one | 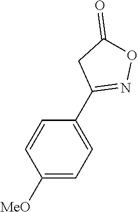 [SMILES: COc1ccc(C2=NOC(=O)C2)cc1] | ¹H NMR (CDCl₃): 7.61 (d, 2H), 6.97 (d, 2H), 3.87 (s, 3H), 3.77 (s, 2H) Reaction performed with NaOAc as base. |

### General Procedure C: Synthesis of Compounds of FormulaI

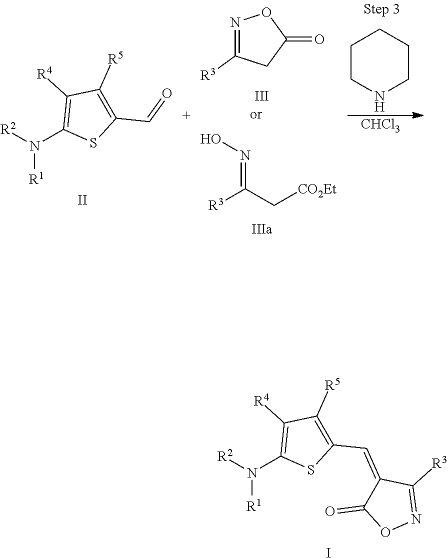

Aminothiophene aldehyde (Formula 11) (1.0 equivalent), was dissolved in CHCl₃ (1.0 M) in a flask or vial, followed by the addition of a solution of 3-substituted-5-isoxazolone or β-oxime ester (Formula III or IIa) (1.3 to 1.5 equivalents if R³═CF₃, CF₂H, CF₂CF₃, CH₃ and 1.0 to 1.1 equivalents if R³=Ph or Ar), in CHCl₃. A catalytic amount of piperidine (0.05 equivalents) was then added. The reaction was heated at reflux, monitoring consumption of aminothiophene aldehyde (Formula I) and formation of product by UV-vis spectroscopy. Upon completion (typically, 48-72 h if R³═CF₃, CF₂H, CF₂CF₃, CH₃ or 24 h if R³=Ph or Ar), the reaction was concentrated via rotary evaporation. If possible, the crude product was precipitated from a mixture of EtOH/hexanes, recovered by vacuum filtration, and washed with EtOH and hexanes. Otherwise, the crude solid was dissolved in CH₂Cl₂ and filtered through a short silica column yielding the desired dyes in yields ranging from 15-80%. Characterization data for the representative compounds are reported in Table 4.

TABLE 4
Representative compounds of Formula I prepared using General Procedure C.

| | | | ¹H NMR | |
|---|---|---|---|---|
| | | | (CDCl₃) | Melting |
| Cmpd | | UV-vis | or MS | Point |
| No. | Structure | (CH₂Cl₂) | (APCI+) | (° C.) |
| 1 | 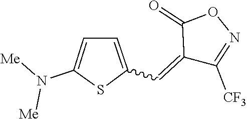 [SMILES: CN(C)c1ccc(C=C2C(=O)ON=C2C(F)(F)F)s1] | λₘₐₓ = 497.8 nm, FWHM = 25.7 nm ε = 97,800 L · mol⁻¹ · cm⁻¹ ε_{g}: 337 L.g⁻¹ · cm⁻¹ | 7.57 (d, 1H), 7.47 (s, 1H), 6.35 (d, 1H), 3.33 (s, 6H) | 205 |
| 2 | 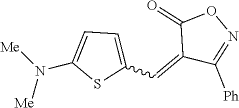 [SMILES: CN(C)c1ccc(C=C2C(=O)ON=C2c2ccccc2)s1] | λₘₐₓ: 498.9 nm FWHM: 33.3 nm ε: 81,500 L · mol⁻¹ · cm⁻¹ ε_{g}: 273 | 7.55-7.62 (m, 2H), 7.49-7.52 (m, 3H), 7.38-7.45 (m, 2H), 6.20 (d, 1H), 3.26 | 142 |
| | | L · g⁻¹ · cm⁻¹ | (s, 6H) | |
| 3 | 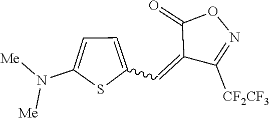 | λₘₐₓ: 498.0 nm FWHM: 26.0 nm | MS (APCI+): 340.9 m/z | — |
| 4 | 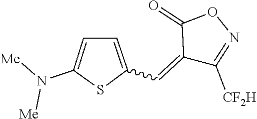 [SMILES: CN(C)c1ccc(C=C2C(=O)ON=C2C(F)F)s1] | λₘₐₓ: 497.1 nm FWHM: 26.8 nm ε: 94,900 L · mol⁻¹ · cm⁻¹ ε_{g}: 349 L · g⁻¹ · cm⁻¹ | 7.66 (s, 1H), 7.56 (d, 1H), 6.50 (t, CF₂H, 1H), 6.30 (d, 1H), 3.31 (s, 6H) MS (APCI+): 273.0 m/z | 207 |
| 5 | 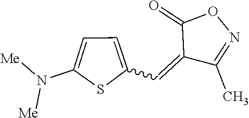 [SMILES: CC1=NOC(=O)C1=Cc1ccc(N(C)C)s1] | λₘₐₓ: 494.4 nm FWHM: 36.6 nm ε: 73,791 L.mol⁻¹ · cm⁻¹ ε_{g}: 312.3 L.g⁻¹ · cm⁻¹ | 7.48 (m, 1H), 7.30 (m, 1H), 6.19 (m, 1H), 3.25 (s, 6H), 2.20 (s, 3H)MS (APCI+): 237.1 m/z | — |
| 6 | 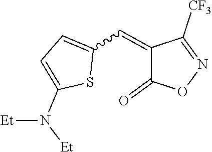 [SMILES: CCN(CC)c1ccc(C=C2C(=O)ON=C2C(F)(F)F)s1] | λₘₐₓ: 500.1 nm FWHM: 25.3 nm ε: 103,800 L · mol⁻¹ · cm⁻¹ ε_{g}: 326 L · g⁻¹ · cm⁻¹ | 7.57 (d, 1H), 7.47 (s, 1H), 6.36 (d, 1H), 3.62 (quart, 4H), 1.36 (t, 6H) | 128 |
| 7 | 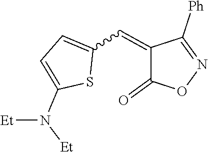 [SMILES: CCN(CC)c1ccc(C=C2C(=O)ON=C2c2ccccc2)s1] | λₘₐₓ: 502.2 nm FWHM: 30.8 nm | 7.54-7.61 (m, 2H), 7.36-7.52 (m, 5H), 6.20 (d, 1H), 3.56 (quart, 4H), 1.31 (t, 6H) | — |
| 8 | 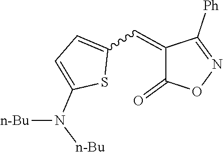 | λₘₐₓ: 504.2 nm FWHM: 30.8 nm | MS (APCI+): 383.1 m/z | — |
| 9 | 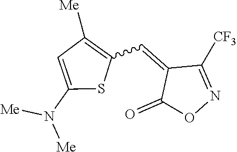 [SMILES: Cc1cc(N(C)C)sc1C=C1C(=O)ON=C1C(F)(F)F] | λₘₐₓ: 498.0 nm FWHM: 26.4 nm | 7.52 (s, 1H), 6.24 (s, 1H), 3.32 (s, 6H), 2.42 (s, 3H) | — |
| 10 | 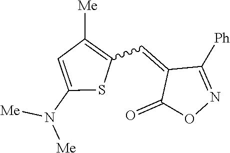 [SMILES: Cc1cc(N(C)C)sc1C=C1C(=O)ON=C1c1ccccc1] | λₘₐₓ: 502.7 nm FWHM: 32.6 nm ε: 83,300 L · mol⁻¹ · cm⁻¹ ε_{g}: 267 L · g⁻¹ · cm⁻¹ | 7.57-7.63 (m, 2H), 7.54 (s, 1H), 7.48-7.52 (m, 3H), 6.09 (s, 1H), 3.25 (s, 6H), 2.29 (s, 3H) | 217 |
| 11 | 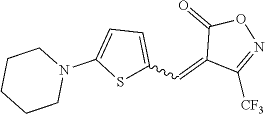 [SMILES: O=C1ON=C(C(F)(F)F)C1=Cc1ccc(N2CCCCC2)s1] | λₘₐₓ: 502.3 nm FWHM: 25.4 nm ε: 101,000 L · mol⁻¹ · cm⁻¹ ε_{g}: 307 L · g⁻¹ · cm⁻¹ | 7.56 (d, 1H), 7.45 (s, 1H), 6.42 (d, 1H), 3.66 (m, 4H), 1.78 (m, 6H) | 202 |
| 12 | 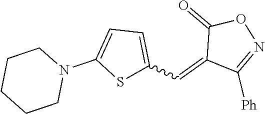 [SMILES: O=C1ON=C(c2ccccc2)C1=Cc1ccc(N2CCCCC2)s1] | λₘₐₓ: 504.2 nm FWHM: 32.8 nm ε: 86,500 L · mol⁻¹ · cm⁻¹ ε_{g}: 256 L · g⁻¹ · cm⁻¹ | 7.46-7.62 (m, 5H), 7.42 (m, 2H), 6.28 (d, 1H), 3.58 (m, 4H), 1.75 (m, 6H) | 169 |
| 13 | 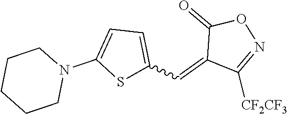 | λₘₐₓ: 502.6 nm FWHM: 25.3 nm ε: 101,600 L · mol⁻¹ · cm⁻¹ ε_{g}: 267 L · g⁻¹ · cm⁻¹ | 7.57 (d, 1H), 7.51 (s, 1H), 6.43 (d, 1H), 3.63-3.70 (m, 4H), 1.75-1.84 (m, 6H) | 179 |
| 14 | 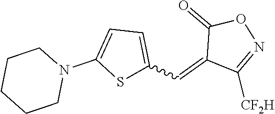 [SMILES: O=C1ON=C(C(F)F)C1=Cc1ccc(N2CCCCC2)s1] | λₘₐₓ: 502.1 nm FWHM: 26.4 nm ε: 108,100 L · mol⁻¹ · cm⁻¹ ε_{g}: 346 L · g⁻¹ · cm⁻¹ | 7.65 (s, 1H), 7.56 (d, 1H), 6.49 (t, 1H), 6.37 (s, 1H), 3.63 (m, 4H), 1.77 (m, 6H) | 158 |
| 15 | 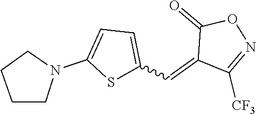 [SMILES: O=C1ON=C(C(F)(F)F)C1=Cc1ccc(N2CCCC2)s1] | λₘₐₓ: 499.9 nm FWHM: 25.1 nm | 7.55 (d, 1H), 7.46 (s, 1H), 6.29 (d, 1H), 3.62 (m, 4H), 2.20 (m, 4H) MS (APCI+): 317.2 m/z | — |
| 16 | 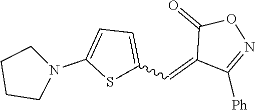 [SMILES: O=C1ON=C(c2ccccc2)C1=Cc1ccc(N2CCCC2)s1] | λₘₐₓ: 502.0 nm FWHM: 29.7 nm | 7.38-7.61 (m, 7H), 6.14 (d, 1H), 3.56 (m, 4H), 2.16 (m, 4H) MS (APCI+): 325.2 m/z | — |
| 17 | 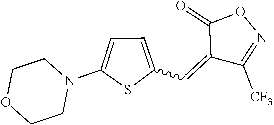 [SMILES: O=C1ON=C(C(F)(F)F)C1=Cc1ccc(N2CCOCC2)s1] | λₘₐₓ: 501.0 nm FWHM: 27.5 nm ε: 99,300 L · mol⁻¹ · cm⁻¹ ε_{g}: 299 L · g⁻¹ · cm⁻¹ | 7.62 (d, 1H), 7.54 (s, 1H), 6.43 (d, 1H), 3.89 (t, 4H), 3.65 (t, 4H) | 226 |
| 18 | 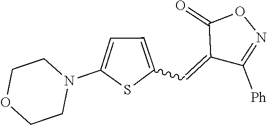 [SMILES: O=C1ON=C(c2ccccc2)C1=Cc1ccc(N2CCOCC2)s1] | λₘₐₓ: 499.3 nm FWHM: 43.4 nm ε: 60,300 L · mol⁻¹ · cm⁻¹ ε_{g}: 177 L · g⁻¹ · cm⁻¹ | 7.37-7.63 (m, 7H), 6.31 (d, 1H), 3.86 (t, 4H), 3.55 (m, 4H) | 179 |
| 19 | 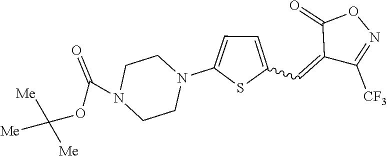 [SMILES: CC(C)(C)OC(=O)N1CCN(c2ccc(C=C3C(=O)ON=C3C(F)(F)F)s2)CC1] | λₘₐₓ: 501.8 nm FWHM: 27.5 nm ε: 99,300 L · mol⁻¹ · cm⁻¹ ε_{g}: 230 L · g⁻¹ · cm⁻¹ | 7.62 (d, 1H), 7.54 (s, 1H), 6.43 (d, 1H), 3.66 (s, 8H), 1.50 (s, 9H) | 200 |
| 20 | 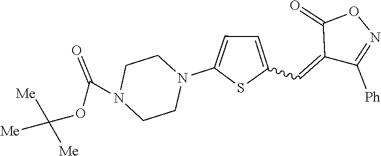 [SMILES: CC(C)(C)OC(=O)N1CCN(c2ccc(C=C3C(=O)ON=C3c3ccccc3)s2)CC1] | λₘₐₓ: 500.2 nm FWHM: 42.3 nm ε: 67,900 L · mol⁻¹ · cm⁻¹ ε_{g}: 155 L · g⁻¹ ^{·} cm⁻¹ | 7.44-7.62 (m, 7H), 6.30 (m, 1H), 3.52-3.66 (m, 8H), 1.49 (s, 9H) | 212 |
| 21 | 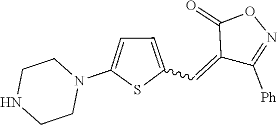 [SMILES: O=C1ON=C(c2ccccc2)C1=Cc1ccc(N2CCNCC2)s1] | λₘₐₓ: 503.0 nm FWHM: 39.6 nm ε: 62,400 L · mol⁻¹ · cm⁻¹ ε_{g}: 184 L · g⁻¹ · cm⁻¹ | 7.39-7.62 (m, 7H), 6.30 (d, 1H) 3.53-3.60 (m, 4H), 3.05 (t, 4H) | 100 |
| 22 | 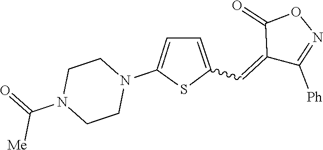 [SMILES: CC(=O)N1CCN(c2ccc(C=C3C(=O)ON=C3c3ccccc3)s2)CC1] | λₘₐₓ: 498.7 nm FWHM: 43.9 nm ε: 61,600 L · mol⁻¹ · cm⁻¹ ε_{g}: 162 L · g⁻¹ · cm⁻¹ | 7.44-7.61 (m, 7H), 6.32 (d, 1H), 3.82 (t, 2H), 3.60-3.71 (m, 4H), 3.56 (t, 2H), 2.18 (s, 3H) | 223 |
| 23 | 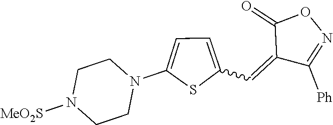 [SMILES: CS(=O)(=O)N1CCN(c2ccc(C=C3C(=O)ON=C3c3ccccc3)s2)CC1] | λₘₐₓ: 496.4 nm FWHM: 47.1 nm ε: 66,200 L · mol⁻¹ · cm⁻¹ ε_{g}: 159 L · g⁻¹ · cm⁻¹ | 7.45-7.61 (m, 7H), 6.32 (d, 1H), 3.69 (t, 4H), 3.43 (t, 4H), 2.86 (s, 3H) | 134 |
| 24 | 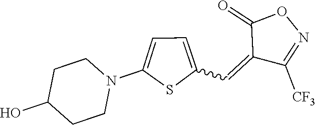 [SMILES: O=C1ON=C(C(F)(F)F)C1=Cc1ccc(N2CCC(O)CC2)s1] | λₘₐₓ: 501.9 nm FWHM: 25.5 nm ε: 104,000 L · mol⁻¹ · cm⁻¹ ε_{g}: 302 L · g⁻¹ · cm⁻¹ | 7.60 (d, 1H), 7.50 (s, 1H), 6.44 (d, 1H), 4.13-4.20 (m, 1H), 3.86-3.93 (m, 2H), 3.58-3.65 (m, 2H), 2.00-2.08 (m, 2H), 1.77-1.87 (m, 2H) | 229 |
| 25 | 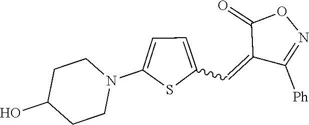 [SMILES: O=C1ON=C(c2ccccc2)C1=Cc1ccc(N2CCC(O)CC2)s1] | λₘₐₓ: 502.8 nm FWHM: 35.4 nm ε: 85,900 L · mol⁻¹ · cm⁻¹ ε_{g}: 242 L · g⁻¹ · cm⁻¹ | 7.38-7.62 (m, 7H), 6.30 (d, 1H) 4.07-4.14 (m, 1H), 3.79-3.89 (m, 2H), 3.43-3.55 (m, 2H), 1.98-2.08 (m, 2H), 1.72-1.83 (m, 2H) | 213 |
| 26 | 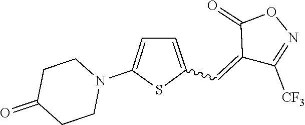 [SMILES: O=C1CCN(c2ccc(C=C3C(=O)ON=C3C(F)(F)F)s2)CC1] | λₘₐₓ: 501.7 nm FWHM: 28.1 nm | MS (APCI+): 344.9 m/z | — |
| 27 | 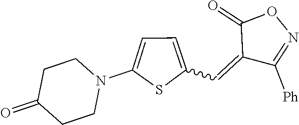 [SMILES: O=C1CCN(c2ccc(C=C3C(=O)ON=C3c3ccccc3)s2)CC1] | λₘₐₓ: 497.5 nm FWHM: 42.4 nm ε: 60,600 L · mol⁻¹ · cm⁻¹ ε_{g}: 172 L · g⁻¹ · cm⁻¹ | 7.47-7.68 (m, 7H), 6.38 (d, 1H), 3.97 (t, 4H), 2.75 (t, 4H) | 155 |
| 28 | 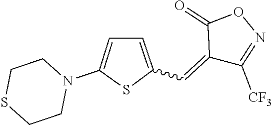 [SMILES: O=C1ON=C(C(F)(F)F)C1=Cc1ccc(N2CCSCC2)s1] | λₘₐₓ: 502.7 nm FWHM: 26.5 nm ε: 101,900 L · mol⁻¹ · cm⁻¹ ε_{g}: 292 L · g⁻¹ · cm⁻¹ | 7.62 (d, 1H), 7.53 (s, 1H), 6.42 (d, 1H), 3.99 (t, 4H), 2.83 (t, 4H) | 249 |
| 29 | 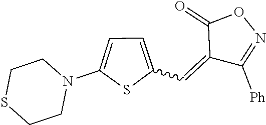 [SMILES: O=C1ON=C(c2ccccc2)C1=Cc1ccc(N2CCSCC2)s1] | λₘₐₓ: 501.9 nm FWHM: 39.3 nm ε: 78,500 L · mol⁻¹ · cm⁻¹ ε_{g}: 220 L · g⁻¹ · cm⁻¹ | 7.42-7.62 (m, 7H), 6.28 (d, 1H), 3.89-3.95 (m, 4H), 2.79 (t, 4H) | 204 |
| 30 | 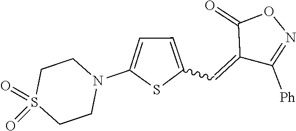 [SMILES: O=C1ON=C(c2ccccc2)C1=Cc1ccc(N2CCS(=O)(=O)CC2)s1] | λₘₐₓ: 488.5 nm FWHM: 53.0 nm | MS (APCI+): 389.1 m/z | — |
| 31 | 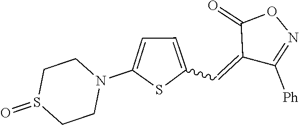 [SMILES: O=C1ON=C(c2ccccc2)C1=Cc1ccc(N2CCS(=O)CC2)s1] | λₘₐₓ: 498.6 nm FWHM: 41.8 nm | MS (APCI+): 373.0 m/z | — |
| 32 | 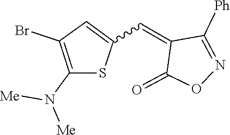 [SMILES: CN(C)c1sc(C=C2C(=O)ON=C2c2ccccc2)cc1Br] | λₘₐₓ: 509.6 nm FWHM: 48.1 nm | — | — |
| 33 | 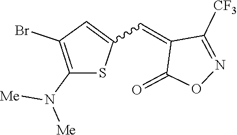 [SMILES: CN(C)c1sc(C=C2C(=O)ON=C2C(F)(F)F)cc1Br] | λₘₐₓ: 515.3 nm FWHM: 30.5 nm | — | — |
| 34 | 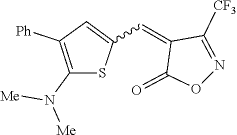 [SMILES: CN(C)c1sc(C=C2C(=O)ON=C2C(F)(F)F)cc1-c1ccccc1] | λₘₐₓ: 509.9 nm FWHM: 32.8 nm | 7.50 (d, 2H), 7.28-7.44 (m, 5H), 3.12 (s, 6H) | — |
| 35 | 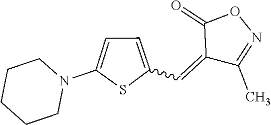 [SMILES: CC1=NOC(=O)C1=Cc1ccc(N2CCCCC2)s1] | λₘₐₓ: 500.0 nm FWHM: 36.8 nm | ¹H NMR (CD₂Cl₂): 7.55 (1H), 7.28 (1H), 6.32 (1H), 3.56 (m, 4H), 2.17 (s, 3H), 1.73 (m, 6H) MS (APCI+): 277.1 m/z | — |
| 36 | 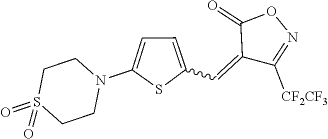 | λₘₐₓ: 496.9 nm FWHM: 32.4 nm | MS (APCI+): 430.9 m/z | — |
| 37 | 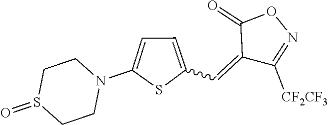 | λₘₐₓ: 498.7 nm FWHM: 27.9 nm | MS (APCI+): 415.0 m/z | — |
| 38 | 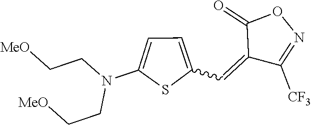 [SMILES: COCCN(CCOC)c1ccc(C=C2C(=O)ON=C2C(F)(F)F)s1] | λₘₐₓ: 500.8 nm FWHM: 26.0 nm | MS (APCI+): 379.0 m/z | — |
| 39 | 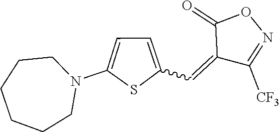 [SMILES: O=C1ON=C(C(F)(F)F)C1=Cc1ccc(N2CCCCCC2)s1] | λₘₐₓ: 501.9 nm FWHM: 26.1 nm | 7.57 (d, 1H), 7.47 (s, 1H), 6.39 (d, 1H), 3.71 (m, 4H), 1.92 (m, 4H), 1.66 (m, 4H) | — |
| 40 | 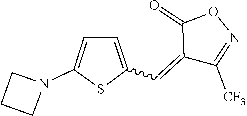 [SMILES: O=C1ON=C(C(F)(F)F)C1=Cc1ccc(N2CCC2)s1] | λₘₐₓ: 500.0 nm FWHM: 24.7 nm | MS (APCI+): 303.1 m/z | — |
| 41 | 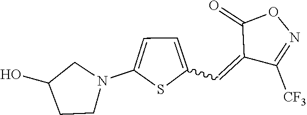 [SMILES: O=C1ON=C(C(F)(F)F)C1=Cc1ccc(N2CCC(O)C2)s1] | λₘₐₓ: 499.4 nm FWHM: 24.9 nm | MS (APCI+): 333.3 m/z | — |
| 42 | 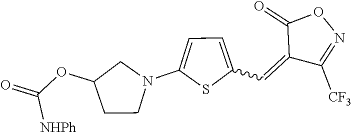 | λₘₐₓ: 499.4 nm FWHM: 25.0 nm | MS (APCI+): 452.5 m/z | — |
| 43 | 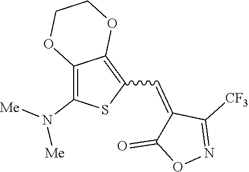 [SMILES: CN(C)c1sc(C=C2C(=O)ON=C2C(F)(F)F)c2c1OCCO2] | λₘₐₓ: 496.6 nm FWHM: 35.4 nm | 7.52 (s, 1H), 4.40-4.45 (m, 2H), 4.25-4.29 (m, 2H), 3.41 (s, 6H) | — |
| 44 | 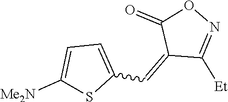 [SMILES: CCC1=NOC(=O)C1=Cc1ccc(N(C)C)s1] | λₘₐₓ: 494.7 nm FWHM: 37.3 nm ε: 73,300 L · mol⁻¹ · cm⁻¹ ε_{g}: 293 L · g⁻¹ · cm⁻¹ | 7.46 (br s, 1H), 7.29 (br s, 1H), 6.18 (br s, 1H), 3.25 (s, 6H), 2.60 (m, 2H), 1.31 (m, 3H) | 199 |
| 45 | 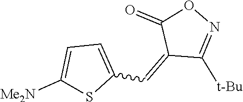 [SMILES: CN(C)c1ccc(C=C2C(=O)ON=C2C(C)(C)C)s1] | λₘₐₓ: 494.0 nm FWHM: 36.3 nm ε: 61,500 L · mol⁻¹ · cm⁻¹ ε_{g}: 221 L · g⁻¹ · cm⁻¹ | 7.66 (s, 1H), 7.46 (br s, 1H), 6.19 (d, 1H), 3.24 (s, 6H), 1.40 (s, 9H) MS (APCI+): 279.2 m/z | 161 |
| 46 | 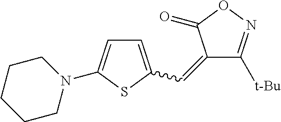 [SMILES: CC(C)(C)C1=NOC(=O)C1=Cc1ccc(N2CCCCC2)s1] | λₘₐₓ: 499.3 nm FWHM: 37.4 nm | 7.64 (s, 1H), 7.27 (m, 1H), 6.28 (d, 1H), 3.56 (m, 4H), 1.73 (m, 6H), 1.40 (s, 9H) MS (APCI+): 319.2 m/z | — |
| 47 | 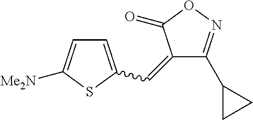 [SMILES: CN(C)c1ccc(C=C2C(=O)ON=C2C2CC2)s1] | λₘₐₓ: 495.0 nm FWHM: 37.7 nm | 7.51 (m, 2H), 6.20 (d, 1H), 3.25 (s, 6H), 1.71 (m, 1H), 1.04 (m, 2H), 0.95 (m, 2H) MS (APCI+): 263.4 m/z | — |
| 48 | 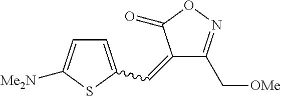 [SMILES: COCC1=NOC(=O)C1=Cc1ccc(N(C)C)s1] | λₘₐₓ: 495.7 nm FWHM: 31.7 nm ε: 83,500 L · mol⁻¹ · cm⁻¹ ε_{g}: 314 L · g⁻¹ · cm⁻¹ | 7.66 (s, 1H), 7.51 (d, 1H), 6.22 (d, 1H), 4.46 (s, 2H), 3.38 (s, 3H), 3.27 (s, 6H) | 153 |
| 49 | 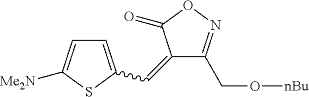 | λₘₐₓ: 495.4 nm FWHM: 32.2 nm | 7.72 (s, 1H), 7.49 (d, 1H), 6.21 (d, 1H), 4.50 (s, 2H), 3.48 (t, 2H), 3.26 (s, 6H), 1.52-1.89 (m, 2H), 1.36 (sextet, 2H), 0.89 | — |
| | | | (t, 3H) | |
| 50 | 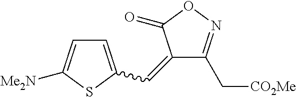 [SMILES: COC(=O)CC1=NOC(=O)C1=Cc1ccc(N(C)C)s1] | λₘₐₓ: 497.1 nm FWHM: 31.6 nm ε: 78,800 L · mol⁻¹ · cm⁻¹ ε_{g}: 268 L · g⁻¹ · cm⁻¹ | 7.50 (m, 1H), 7.40 (s, 1H), 6.23 (d, 1H), 3.74 (s, 3H), 3.64 (s, 2H), 3.25 (s, 6H) | 193 |
| 51 | 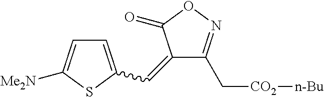 | λₘₐₓ: 497.1 nm FWHM: 32.1 nm | 7.48 (d, 1H), 7.41 (s, 1H), 6.22 (d, 1H), 4.13 (t, 2H), 3.62 (s, 2H), 3.26 (s, 6H), 1.60 (quint, 2H), 1.35 (sextet, 2H), 0.89 (t, 3H) | — |
| 52 | 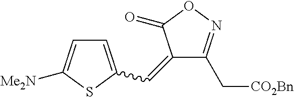 [SMILES: CN(C)c1ccc(C=C2C(=O)ON=C2CC(=O)OCc2ccccc2)s1] | λₘₐₓ: 497.2 nm FWHM: 32.1 nm | 7.28-7.37 (m, 7H), 6.19 (d, 1H), 5.17 (s, 2H), 3.67 (s, 2H), 3.26 (s, 6H) | — |
| 53 | 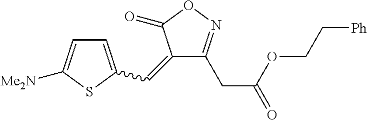 [SMILES: CN(C)c1ccc(C=C2C(=O)ON=C2CC(=O)OCCc2ccccc2)s1] | λₘₐₓ: 497.0 nm FWHM: 31.5 nm | 7.40 (s, 1H), 7.35 (d, 1H), 7.27 (t, 2H), 6.96 (t, 1H), 6.86 (d, 2H), 6.11 (d, 1H), 4.50 (t, 2H), 4.17 (t, 2H), 3.69 (s, 2H), 3.25 (s, 6H) | — |
| 54 | 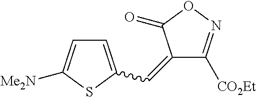 [SMILES: CCOC(=O)C1=NOC(=O)C1=Cc1ccc(N(C)C)s1] | λₘₐₓ: 503.4 nm FWHM: 25.7 nm ε: 101,700 L · mol⁻¹ · cm⁻¹ ε_{g}: 346 L · g⁻¹ · cm⁻¹ | 8.33 (s, 1H), 7.60 (d, 1H), 6.31 (d, 1H), 4.43 (quart, 2H), 3.30 (s, 6H), 1.43 (t, 3H) MS (APCI+): 295.2 m/z | 186 |
| 55 | 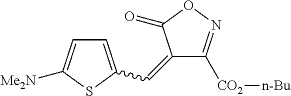 | λₘₐₓ: 503.4 nm FWHM: 26.6 nm | MS (ACPI+): 323.6 m/z | — |
| 56 | 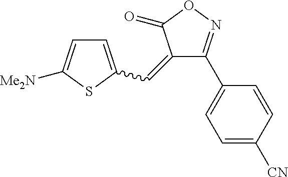 [SMILES: CN(C)c1ccc(C=C2C(=O)ON=C2c2ccc(C#N)cc2)s1] | λₘₐₓ: 500.5 nm FWHM: 31.7 nm | — | — |
| 57 | 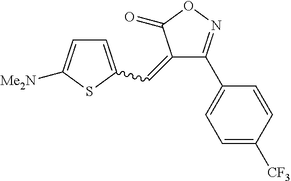 [SMILES: CN(C)c1ccc(C=C2C(=O)ON=C2c2ccc(C(F)(F)F)cc2)s1] | λₘₐₓ: 500.2 nm FWHM: 30.9 nm | 7.78 (d, 2H), 7.72 (d, 2H), 7.44 (d, 1H), 7.37 (s, 1H), 6.24 (d, 1H), 3.24 (s, 6H) MS (APCI+): 367.5 m/z | — |
| 58 | 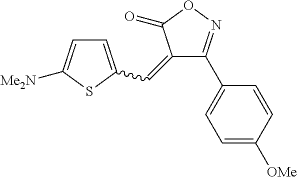 [SMILES: COc1ccc(C2=NOC(=O)C2=Cc2ccc(N(C)C)s2)cc1] | λₘₐₓ: 498.9 nm FWHM: 35.5 nm | 7.53 (d, 2H), 7.42 (m, 2H), 7.03 (d, 2H), 6.19 (d, 1H), 3.87 (s, 3H), 3.26 (s, 6H) MS (APCI+): 329.4 m/z | — |
| 59 | 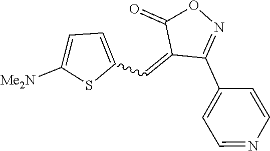 [SMILES: CN(C)c1ccc(C=C2C(=O)ON=C2c2ccncc2)s1] | λₘₐₓ: 500.8 nm FWHM: 30.1 nm | 8.79 (d, 2H), 7.53 (d, 2H), 7.47 (d, 1H), 7.40 (s, 1H), 6.26 (d, 1H), 3.30 (s, 6H) | — |
| 60 | 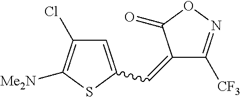 [SMILES: CN(C)c1sc(C=C2C(=O)ON=C2C(F)(F)F)cc1Cl] | λₘₐₓ: 514.8 nm FWHM: 32.3 nm | MS (APCI+): 325.1 m/z | — |

### General Procedure D: Process for Preparing Lenses Comprising Compounds of Formula I

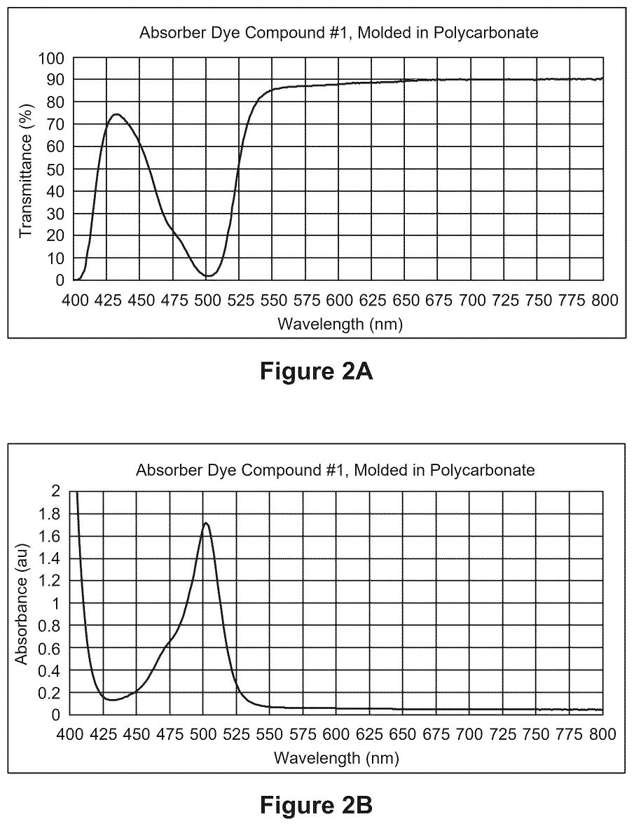

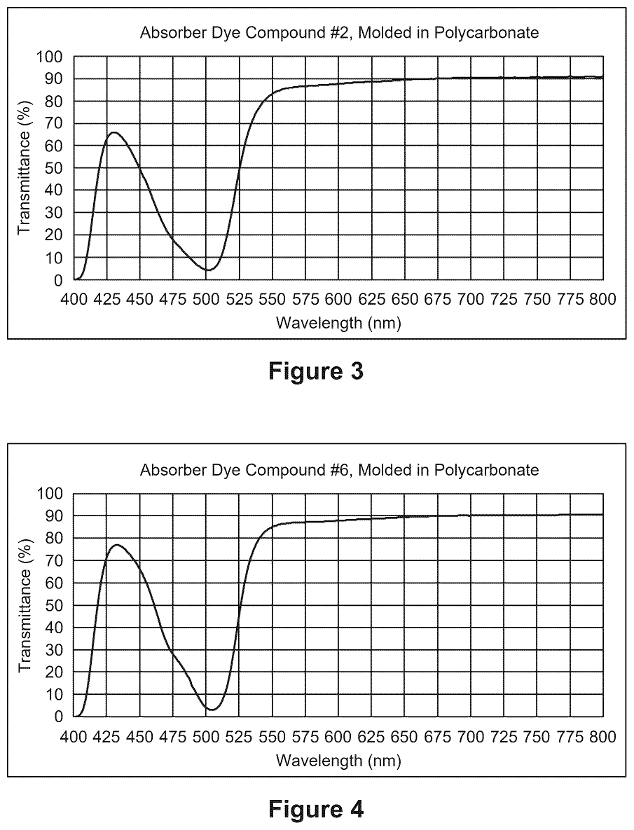

4-((5-(dimethylamino)thiophen-2-yl)methylene)-3-(trifluoromethyl)isoxazol-5(4H)-one (Compound 1) was formulated into resin lenses for the purposes of chroma-enhancement. For example, a blend of Compound 1 (34 mg) and polycarbonate resin (3 pounds) were blended and injected molded at 500° F., producing a 2 mm thick lens with an optical density between about 1.7 and 1.8. The resulting lens containing Compound 1 was analyzed by UV-vis spectroscopy, producing the non-normalized transmission and absorption spectra shown in FIGS. 2A and 2B, respectively. Additional transmission spectra for molded polycarbonate lenses containing dyes Compound 2 (42 mg per 3 pounds of resin) and Compound 6 (35 mg dye per 3 pounds of resin) are shown in FIGS. 3 and 4, respectively.

### Example 2: Synthesis of 4-((5-(4-oxopiperidin-1-yl)thiophen-2-yl)methylene)-3-phenylisoxazol-5(4H)-one (Compound 27)

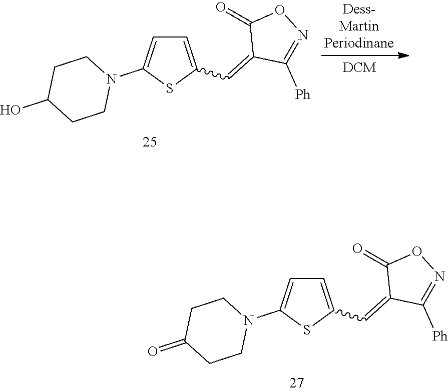 [SMILES: O=C1ON=C(c2ccccc2)C1=Cc1ccc(N2CCC(O)CC2)s1 ; O=C1CCN(c2ccc(C=C3C(=O)ON=C3c3ccccc3)s2)CC1]

4-((5-(4-hydroxypiperidin-1-yl)thiophen-2-yl)methylene)-3-phenylisoxazol-5(4H)-one (40 mg, 1.0 equivalents) was added to wet CH₂Cl₂. The resulting suspension was treated with Dess-Martin Periodinane (70 mg, 1.5 equivalents). The mixture was stirred at room temperature for 30 min. Upon full consumption of 4-((5-(4-hydroxypiperidin-1-yl)thiophen-2-yl)methylene)-3-phenylisoxazol-5(4H)-one as monitored by TLC, the reaction was diluted with CH₂Cl₂ and washed with H₂O. The organic layer was dried with Na₂SO₄, filtered, and concentrated by rotary evaporation. The crude residue was purified by silica gel chromatography (100% CH₂Cl₂ to 10% EtOAc in CH₂Cl₂). The fractions containing the product where concentrated to provide 4-((5-(4-oxopiperidin-1-yl)thiophen-2-yl)methylene)-3-phenylisoxazol-5(4H)-one (25 mg, 63% yield). ¹H NMR (CDCl₃): 7.47-7.68 (m, 7H), 6.38 (d, 1H), 3.97 (t, 4H), 2.75 (t, 4H).

### Example 3: Synthesis of 5-(4-oxopiperidin-1-yl)thiophene-2-carbaldehyde (Compound 178)

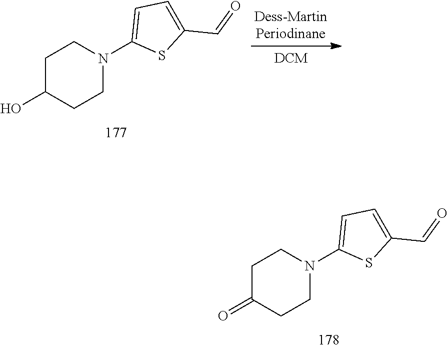 [SMILES: O=Cc1ccc(N2CCC(O)CC2)s1 ; O=Cc1ccc(N2CCC(=O)CC2)s1]

5-(4-hydroxypiperidin-1-yl)thiophene-2-carbaldehyde (150 mg, 1.0 equiv) was added to wet CH₂Cl₂. The resulting suspension was treated with Dess-Martin Periodinane (320 mg, 1.1 equiv). The mixture was stirred at room temperature overnight. After 20 hours, full conversion of 5-(4-hydroxypiperidin-1-yl)thiophene-2-carbaldehyde was observed by TLC. The reaction was diluted with CH₂C₂ and washed with H₂O. The organic layer was dried with Na₂SO₄, filtered, and concentrated by rotary evaporation. The crude residue was purified by silica gel chromatography (100% CH₂C₂ to 10% EtOAc in CH₂Cl₂). The fractions containing the product were concentrated to provide 5-(4-oxopiperidin-1-yl)thiophene-2-carbaldehyde (105 mg, 71% yield). ¹H NMR (CDCl₃): 9.59 (s, 1H), 7.55 (d, 1H), 6.18 (d, 1H), 3.76 (t, 4H), 2.65 (t, 4H).

### Example 4: Synthesis of 5-(4-acetylpiperazin-1-yl)thiophene-2-carbaldehyde (Compound 179)

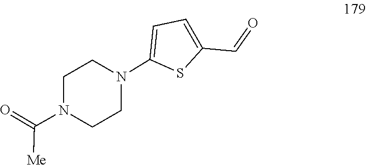 [SMILES: CC(=O)N1CCN(c2ccc(C=O)s2)CC1]

5-(piperazin-1-yl)thiophene-2-carbaldehyde (100 mg, 1.0 equiv) was added to CH₂C₂ (2.0 mL). The resulting suspension was stirred at room temperature followed by the addition of acetyl chloride (50 μL, 1.4 equiv) and triethylamine (169 μL, 2.4 equiv). After 90 min, full conversion of 5-(piperazin-1-yl)thiophene-2-carbaldehyde was observed by TLC. The reaction was diluted in CH₂Cl₂ and washed with Na₂CO₃ (aq) and H₂O. Silica and Na₂CO₃ were added to the organic layer. The resulting slurry was filtered and washed with CH₂Cl₂. The filtrate was concentrated by rotary evaporation, providing 5-(4-acetylpiperazin-1-yl)thiophene-2-carbaldehyde (69 mg, 57% yield). ¹H NMR (CDCl₃): 9.58 (s, 1H), 7.52 (d, 2H), 6.16 (d, 1H), 3.79 (t, 2H) 3.65 (t, 2H), 3.37 (dt, 4H), 2.15 (s, 3H).

### Example 5: Synthesis of 5-(4-(methylsulfonyl)piperazin-1-yl)thiophene-2-carbaldehyde) (Compound 180)

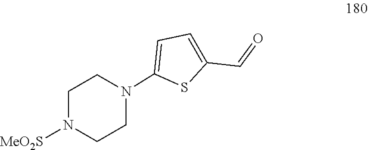 [SMILES: CS(=O)(=O)N1CCN(c2ccc(C=O)s2)CC1]

5-(piperazin-1-yl)thiophene-2-carbaldehyde (100 mg, 1.0 equiv) was added to CH₂Cl₂ (2.0 mL). The resulting suspension was stirred at room temperature followed by the addition of methanesulfonyl chloride (55 μL, 1.4 equiv) and triethylamine (169 μL, 2.4 equiv). After 90 min, full conversion of 5-(piperazin-1-yl)thiophene-2-carbaldehyde was observed by TLC. The reaction was diluted in CH₂Cl₂ and washed with Na₂CO₃ (aq) and H₂O. Silica and Na₂CO₃ were added to the organic layer. The resulting slurry was filtered and washed with CH₂Cl₂. The filtrate was concentrated by rotary evaporation, providing 5-(4-(methylsulfonyl)piperazin-1-yl)thiophene-2-carbaldehyde (44 mg, 31% yield). 1H NMR (CDCl₃): 9.60 (s, 1H), 7.52 (d, 1H), 6.17 (d, 1H), 3.36-3.52 (m, 8H), 2.84 (s, 3H).

### Example 6: Synthesis of 5-(1-oxidothiomorpholino)thiophene-2-carbaldehyde and 5-(1,1-dioxidothiomorpholino)thiophene-2-carbaldehyde (Compound 182 and Compound 183)

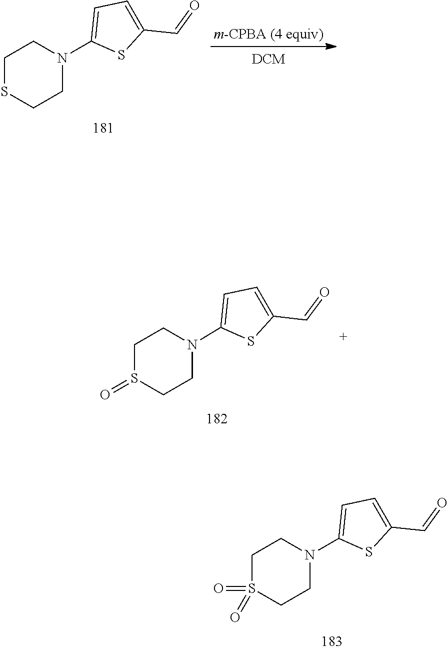 [SMILES: O=Cc1ccc(N2CCSCC2)s1 ; O=Cc1ccc(N2CCS(=O)CC2)s1 ; O=Cc1ccc(N2CCS(=O)(=O)CC2)s1]

A mixture of 5-thiomorpholinothiophene-2-carbaldehyde (150 mg, 1 equiv) in CH₂Cl₂ (3.0 mL) was stirred at room temperature followed by the addition of m-CPBA (485 mg, 4 equiv) The reaction was stirred for 20 h at room temperature. The reaction was diluted with CH₂Cl₂ and washed with Na₂CO₃(aq). The organic layer was concentrated by rotary evaporation. The resulting crude residue was diluted in CH₂Cl₂ and filtered through a silica plug using CH₂Cl₂/EtOH. The filtrate was concentrated by rotary evaporation, resulting in a 1:1 mixture of 5-(1-oxidothiomorpholino)thiophene-2-carbaldehyde and 5-(1,1-dioxidothiomorpholino)thiophene-2-carbaldehyde by 1H NMR (CDCl₃). The mixture was used without further purification. ¹H NMR (CDCl₃) of mixture: 9.63 (s, 1H), 9.60 (s, 1H), 7.52 (m, 2H), 6.19 (m, 2H), 4.24 (t, 2H), 3.98 (m, 4H), 3.69 (m, 2H), 3.17 (m, 4H), 2.88 (m, 4H).

### Example 7: Synthesis of 4-bromo-5-(dimethylamino)thiophene-2-carbaldehyde (Compound 184)

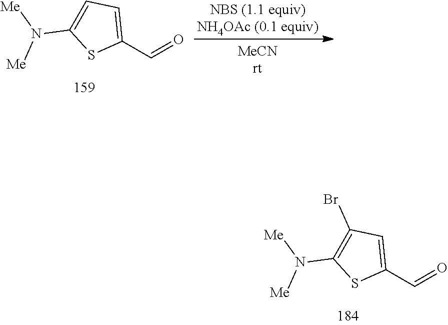 [SMILES: CN(C)c1ccc(C=O)s1 ; CN(C)c1sc(C=O)cc1Br]

In a vial, 5-(dimethylamino)thiophene-2-carbaldehyde (1.0 g, 1 equiv) was dissolved in MeCN (26 mL, 0.25M). Solid NH₄OAc (50 mg, 0.1 equiv) and N-bromosuccinimide (1.26 g, 1.1 equiv) were then added to the reaction. The mixture was stirred at room temperature for 1.5 h. The reaction was monitored by TLC. Upon completion, the reaction was quenched with a solution of sodium thiosulfate solution in H₂O (added dropwise). The product was extracted with CH₂Cl₂. The organic layer washed with H₂O. The organic layer was concentrated to dryness by rotary evaporation. The crude residue was dissolved in CH₂Cl₂ and slurried with silica. The slurry was filtered and the silica was washed with CH₂Cl₂. The filtrate was concentrated to provide 4-bromo-5-(dimethylamino)thiophene-2-carbaldehyde as an amber oil (1.38 g, 91% yield). ¹H NMR (CDCl₃): 9.54 (s, 1H), 7.49 (s, 1H), 3.13 (s, 6H). Replacing N-bromosuccinimide with N-chlorosuccinimide, the procedure provided 4-chloro-5-(dimethylamino)thiophene-2-carbaldehyde (Compound 186) (104 mg, 55% yield). ¹H NMR (CDCl₃): 9.52 (s, 1H), 7.42 (s, 1H), 3.14 (s, 6H).

### Example 8: Synthesis of 5-(dimethylamino)-4-phenylthiophene-2-carbaldehyde (Compound 154)

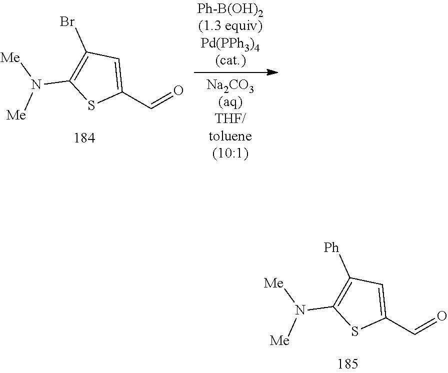 [SMILES: CN(C)c1sc(C=O)cc1Br ; CN(C)c1sc(C=O)cc1-c1ccccc1]

In a vial a solution of THF/toluene mixture (10:1, 1.9 mL) and Na₂CO₃ (0.5 mL, 2.0 M aqueous solution, 2.2 equiv) was degassed with N₂. Then solid 4-bromo-5-(dimethylamino)thiophene-2-carbaldehyde (100 mg, 1.0 equiv), phenylboronic acid (68 mg, 1.3 equiv), and Pd(PPh₃)₄ (5 mg) were added to the reaction. The reaction was placed under N₂ and heated at 80° C. The reaction was monitored by TLC. After 26 h, the reaction mixture was extracted with CH₂Cl₂. The organic layer was dried with sodium sulfate, filtered, and concentrated by rotary evaporation. The crude product was purified by silica gel chromatography (40 g column, 15 μm silica, 0-20% EtOAc in hexanes). The fractions containing product were concentrated by rotary evaporation to provide 5-(dimethylamino)-4-phenylthiophene-2-carbaldehyde as an oil (15 mg, 15% yield). ¹H NMR (CDCl₃): 9.58 (s, 1H), 7.56 (s, 1H), 7.28-7.46 (m, 5H), 2.86 (s, 6H).

It is to be appreciated that the Detailed Description section, and not the Summary and Abstract sections, is intended to be used to interpret the claims. The Summary and Abstract sections may set forth one or more but not all exemplary aspects of the present invention as contemplated by the inventor(s), and thus, are not intended to limit the present invention and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the invention that others can, by applying knowledge within the skill of the art, readily modify and/or adapt for various applications such specific aspects, without undue experimentation, without departing from the general concept of the present invention. For example, the compounds described herein can be incorporated into a translucent visor brim which can be positioned above the wearer's eyes and wearer's nose to render chroma-enhancement properties, such visor application being described in U.S. Provisional Application No. 63/386,486. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary aspects, but should be defined only in accordance with the following claims and their equivalents.

What is claimed is:

1. A compound of Formula I 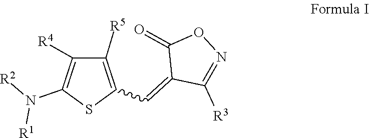
wherein:
R¹ and R² are each, independently, C₁-C₆ alkyl, C₁-C₆ alkylene-OH, alkylene C(O)OR⁶, alkylene nitrile, C₁-C₆ haloalkyl, SO₂-haloalkyl, C₁-C₆ alkylene-O-alkyl, alkylene OC(O)NHR⁷, or C₃-C₆ cycloalkyl;
or R¹ and R² are taken together with the nitrogen to which they are bound to form an optionally substituted 4- to 7-membered heterocycle;
R³ is C₁-C₆alkyl, C₁-C₆ haloalkyl, C₁-C₆ alkenyl, C₁-C₆ alkynyl, C₁-C₆ alkoxy, SO₂-alkyl, SO₂-haloalkyl, SO₂-aryl, C₁-C₆ cycloalkyl, C₁-C₆ alkylene-O-alkyl, nitrile, C(O)OR⁶, optionally substituted C₆-C₁₀ aryl, or optionally substituted C₃-C₉ heteroaryl;
R⁴ and R⁵ are each, independently, H, D, halo, C₁-C₆ alkyl, C₁-C₆ alkoxy, C₁-C₆ alkenyl, C₁-C₆ alkynyl, C₁-C₆ cycloalkyl, or C₆-C₁₀ aryl; or R⁴ and R⁵ can be taken together with the ring to which they are bound form an optionally substituted C₆-C₁₀ cycloalkyl optionally containing 1-3 heteroatoms;
R⁶ is C₁-C₆ alkyl; and
R⁷ can be H, alkyl, haloalkyl, optionally substituted phenyl, benzyl or phenylalkyl;
provided that:
when R¹ and R² are both ethyl, R³ is not phenyl.

2. The compound of claim 1, wherein
R¹ and R² are each, independently, methyl, ethyl, n-propyl, n-butyl, (CH₂)ₘCO₂R⁶, (CH₂)ₘCN, (CH₂)ₘCF₃, or C₃-C₆ cycloalkyl;
or R¹ and R² are taken together with the nitrogen to which they are bound to form a 4- to 7-membered heterocycle, wherein the 4- to 7-membered heterocycle is 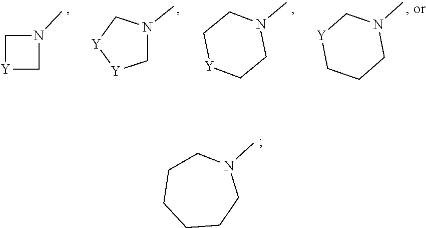
R³ is methyl, ethyl, t-butyl, methoxy, ethoxy, CF₃, CF₂H, CH₂F, CF₂CF₃, CH₂Cl, CCl₃, CF₂Cl, CFCl₂, phenyl, pyridin-4-yl, pyridin-3-yl, pyridine-2-yl, or 4-methoxyphenyl;
R⁴ and R⁵ are each, independently, selected from H, D, methyl, ethyl, i-propyl, t-butyl, n-butyl, halo, methoxy, ethoxy, cycloalkyl, or phenyl;
R⁶ is C₁-C₆ alkyl;
each m is, independently, 1, 2, 3, or 4; and
Y is, independently, CH₂, O, S, SO, SO₂, CF₂, NR⁷, N—C(O)R⁷, N—SO₂—R⁷, N—C(O)OR⁷, N—C(O)N(R⁷)₂, CO, C(H)OH, C(H)OR⁷, C(H)CN, C(H)OC(O)NHR⁷, C(H)CO₂R⁷, C(H)CF₃, C(H)-alkyl, or C(H)-aryl; and
R⁷ can be H, alkyl, haloalkyl, phenyl, benzyl or phenylalkyl.

3. The compound of claim 2, wherein
R¹ and R² are each, independently, methyl, ethyl, or n-butyl;
R³ is methyl, ethyl, CF₃, CF₂H, CF₂CF₃, or phenyl; and
R⁴ and R⁵ are each, independently, H, methyl, halo, or phenyl or R⁴ and R⁵ can be taken together with the ring to which they are bound to form an optionally substituted C₆-C₁₀ cycloalkyl optionally containing 1-3 heteroatoms.

4. The compound of claim 2, wherein
R¹ and R² are taken together with the nitrogen to which they are bound to form the 6-member heterocycle or; 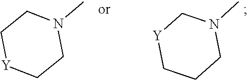
R³ is CF₃ or phenyl;
R⁴ and R⁵ are H; and
Y is C(H)OH or CO.

5. The compound of claim 2, wherein
R¹ and R² are taken together with the nitrogen to which they are bound to form the 6-member heterocycle 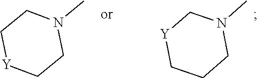
R³ is CF₃ or phenyl;
R⁴ and R⁵ are H;
Y is N—C(O)R⁷, N—C(O)OR⁷ or N—SO₂—R⁷; and
R⁷ is methyl or t-butyl.

6. The compound of claim 2, wherein
R¹ and R² are taken together with the nitrogen to which they are bound to form the 6-member heterocycle 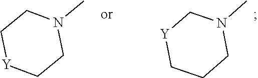
R³ is CF₃ or phenyl;
R⁴ and R⁵ are H; and
Y is O, SO, or SO₂.

7. The compound of claim 2, wherein
R¹ and R² are taken together with the nitrogen to which they are attached to form the 5-member or 6-member heterocycle 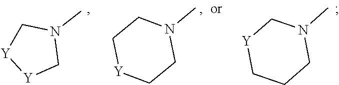
R³ is methyl, CF₃ or phenyl; and
R⁴ and R⁵ are H;
Y is CH₂.

8. The compound of claim 2, wherein:
R¹ and R² are each, independently, methyl, ethyl, or n-butyl;
or R¹ and R² are taken together with the nitrogen to which they are bound to form the 4- to 7-membered heterocycle 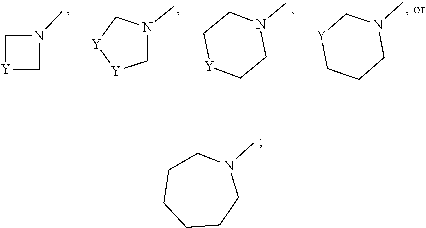
R³ is methyl, CF₃, CF₂H, CF₂CF₃, or phenyl; and
R⁴ and R⁵ are each, independently, H, methyl, bromo, or phenyl;
Y in each instance is, independently, CH₂, O, S, SO, SO₂, C(H)OH, CO, N—C(O)CH₃, N—C(O)O-t-butyl, N—C(O)OCH₃ or N—SO₂—CH₃.

9. The compound of claim 1 wherein the compound is
4-((5-(dimethylamino)thiophen-2-yl)methylene)-3-(trifluoromethyl)isoxazol-5(4H)-one;
4-((5-(dimethylamino)thiophen-2-yl)methylene)-3-phenylisoxazol-5(4H)-one;
4-((5-(dimethylamino)thiophen-2-yl)methylene)-3-(perfluoroethyl)isoxazol-5(4H)-one;
3-(difluoromethyl)-4-((5-(dimethylamino)thiophen-2-yl)methylene)isoxazol-5(4H)-one;
4-((5-(dimethylamino)thiophen-2-yl)methylene)-3-methylisoxazol-5(4H)-one;
4-((5-(diethylamino)thiophen-2-yl)methylene)-3-(trifluoromethyl)isoxazol-5(4H)-one;
4-((5-(diethylamino)thiophen-2-yl)methylene)-3-phenylisoxazol-5(4H)-one;
4-((5-(dibutylamino)thiophen-2-yl)methylene)-3-phenylisoxazol-5(4H)-one;
4-((5-(dimethylamino)-3-methylthiophen-2-yl)methylene)-3-(trifluoromethyl)isoxazol-5(4H)-one;
4-((5-(dimethylamino)-3-methylthiophen-2-yl)methylene)-3-phenylisoxazol-5(4H)-one;
4-((5-(piperidin-1-yl)thiophen-2-yl)methylene)-3-(trifluoromethyl)isoxazol-5(4H)-one;
3-phenyl-4-((5-(piperidin-1-yl)thiophen-2-yl)methylene)isoxazol-5(4H)-one;
3-(perfluoroethyl)-4-((5-(piperidin-1-yl)thiophen-2-yl)methylene)isoxazol-5(4H)-one;
3-(difluoromethyl)-4-((5-(piperidin-1-yl)thiophen-2-yl)methylene)isoxazol-5(4H)-one;
4-((5-(pyrrolidin-1-yl)thiophen-2-yl)methylene)-3-(trifluoromethyl)isoxazol-5(4H)-one;
3-phenyl-4-((5-(pyrrolidin-1-yl)thiophen-2-yl)methylene)isoxazol-5(4H)-one;
4-((5-morpholinothiophen-2-yl)methylene)-3-(trifluoromethyl)isoxazol-5(4H)-one;
4-((5-morpholinothiophen-2-yl)methylene)-3-phenylisoxazol-5(4H)-one;
tert-butyl-4-(5-((5-oxo-3-(trifluoromethyl)isoxazol-4(5H)-ylidene)methyl)thiophen-2-yl)piperazine-1-carboxylate;
tert-butyl-4-(5-((5-oxo-3-phenylisoxazol-4(5H)-ylidene)methyl)thiophen-2-yl)piperazine-1-carboxylate;
3-phenyl-4-((5-(piperazin-1-yl)thiophen-2-yl)methylene)isoxazol-5(4H)-one;
4-((5-(4-acetylpiperazin-1-yl)thiophen-2-yl)methylene)-3-phenylisoxazol-5(4H)-one;
4-((5-(4-(methylsulfonyl)piperazin-1-yl)thiophen-2-yl)methylene)-3-phenylisoxazol-5(4H)-one;
4-((5-(4-hydroxypiperidin-1-yl)thiophen-2-yl)methylene)-3-(trifluoromethyl)isoxazol-5(4H)-one;
4-((5-(4-hydroxypiperidin-1-yl)thiophen-2-yl)methylene)-3-phenylisoxazol-5(4H)-one;
4-((5-(4-oxopiperidin-1-yl)thiophen-2-yl)methylene)-3-(trifluoromethyl)isoxazol-5(4H)-one;
4-((5-(4-oxopiperidin-1-yl)thiophen-2-yl)methylene)-3-phenylisoxazol-5(4H)-one;
4-((5-thiomorpholinothiophen-2-yl)methylene)-3-(trifluoromethyl)isoxazol-5(4H)-one;
3-phenyl-4-((5-thiomorpholinothiophen-2-yl)methylene)isoxazol-5(4H)-one;
4-((5-(1,1-dioxidothiomorpholino)thiophen-2-yl)methylene)-3-phenylisoxazol-5(4H)-one;
4-((5-(1-oxidothiomorpholino)thiophen-2-yl)methylene)-3-phenylisoxazol-5(4H)-one;
4-((4-bromo-5-(dimethylamino)thiophen-2-yl)methylene)-3-(trifluoromethyl)isoxazol-5(4H)-one;
4-((4-bromo-5-(dimethylamino)thiophen-2-yl)methylene)-3-phenylisoxazol-5(4H)-one;
4-((5-(dimethylamino)-4-phenylthiophen-2-yl)methylene)-3-(trifluoromethyl)isoxazol-5(4H)-one;
3-methyl-4-((5-(piperidin-1-yl)thiophen-2-yl)methylene)isoxazol-5(4H)-one;
4-((5-(1,1-dioxidothiomorpholino)thiophen-2-yl)methylene)-3-(perfluoroethyl)isoxazol-5(4H)-one;
4-((5-(1-oxidothiomorpholino)thiophen-2-yl)methylene)-3-(perfluoroethyl)isoxazol-5(4H)-one;
4-((5-(bis(2-methoxyethyl)amino)thiophen-2-yl)methylene)-3-(trifluoromethyl)isoxazol-5(4H)-one;
4-((5-(azepan-1-yl)thiophen-2-yl)methylene)-3-(trifluoromethyl)isoxazol-5(4H)-one;
4-((5-(azetidin-1-yl)thiophen-2-yl)methylene)-3-(trifluoromethyl)isoxazol-5(4H)-one;
4-((5-(3-hydroxypyrrolidin-1-yl)thiophen-2-yl)methylene)-3-(trifluoromethyl)isoxazol-5(4H)-one;
1-(5-((5-oxo-3-(trifluoromethyl)isoxazol-4(5H)-ylidene)methyl)thiophen-2-yl)pyrrolidin-3-yl phenylcarbamate;
4-((7-(dimethylamino)-2,3-dihydrothieno[3,4-b][1,4]dioxin-5-yl)methylene)-3-(trifluoromethyl)isoxazol-5(4H)-one;
4-((5-(dimethylamino)thiophen-2-yl)methylene)-3-ethylisoxazol-5(4H)-one;
3-(tert-butyl)-4-((5-(dimethylamino)thiophen-2-yl)methylene)isoxazol-5(4H)-one;
3-(tert-butyl)-4-((5-(piperidin-1-yl)thiophen-2-yl)methylene)isoxazol-5(4H)-one;
3-cyclopropyl-4-((5-(dimethylamino)thiophen-2-yl)methylene)isoxazol-5(4H)-one;
4-((5-(dimethylamino)thiophen-2-yl)methylene)-3-(methoxymethyl)isoxazol-5(4H)-one;
3-(butoxymethyl)-4-((5-(dimethylamino)thiophen-2-yl)methylene)isoxazol-5(4H)-one;
methyl 2-(4-((5-(dimethylamino)thiophen-2-yl)methylene)-5-oxo-4,5-dihydroisoxazol-3-yl)acetate;
butyl 2-(4-((5-(dimethylamino)thiophen-2-yl)methylene)-5-oxo-4,5-dihydroisoxazol-3-yl)acetate;
benzyl 2-(4-((5-(dimethylamino)thiophen-2-yl)methylene)-5-oxo-4,5-dihydroisoxazol-3-yl)acetate;
phenethyl 2-(4-((5-(dimethylamino)thiophen-2-yl)methylene)-5-oxo-4,5-dihydroisoxazol-3-yl)acetate;
ethyl 4-((5-(dimethylamino)thiophen-2-yl)methylene)-5-oxo-4,5-dihydroisoxazole-3-carboxylate;
butyl 4-((5-(dimethylamino)thiophen-2-yl)methylene)-5-oxo-4,5-dihydroisoxazole-3-carboxylate;
4-(4-((5-(dimethylamino)thiophen-2-yl)methylene)-5-oxo-4,5-dihydroisoxazol-3-yl)benzonitrile;
4-((5-(dimethylamino)thiophen-2-yl)methylene)-3-(4-(trifluoromethyl)phenyl)isoxazol-5(4H)-one;
4-((5-(dimethylamino)thiophen-2-yl)methylene)-3-(4-methoxyphenyl)isoxazol-5(4H)-one;
4-((5-(dimethylamino)thiophen-2-yl)methylene)-3-(pyridin-4-yl)isoxazol-5(4H)-one; or
4-((4-chloro-5-(dimethylamino)thiophen-2-yl)methylene)-3-(trifluoromethyl)isoxazol-5(4H)-one.

10. The compound of claim 1 wherein the compound is
(Z)-4-((5-(dimethylamino)thiophen-2-yl)methylene)-3-(trifluoromethyl)isoxazol-5(4H)-one;
(Z)-4-((5-(dimethylamino)thiophen-2-yl)methylene)-3-phenylisoxazol-5(4H)-one;
(Z)-4-((5-(dimethylamino)thiophen-2-yl)methylene)-3-(perfluoroethyl)isoxazol-5(4H)-one;
(Z)-3-(difluoromethyl)-4-((5-(dimethylamino)thiophen-2-yl)methylene)isoxazol-5(4H)-one;
(Z)-4-((5-(dimethylamino)thiophen-2-yl)methylene)-3-methylisoxazol-5(4H)-one;
(Z)-4-((5-(diethylamino)thiophen-2-yl)methylene)-3-(trifluoromethyl)isoxazol-5(4H)-one;
(Z)-4-((5-(diethylamino)thiophen-2-yl)methylene)-3-phenylisoxazol-5(4H)-one;
(Z)-4-((5-(dibutylamino)thiophen-2-yl)methylene)-3-phenylisoxazol-5(4H)-one;
(Z)-4-((5-(dimethylamino)-3-methylthiophen-2-yl)methylene)-3-(trifluoromethyl)isoxazol-5(4H)-one;
(Z)-4-((5-(dimethylamino)-3-methylthiophen-2-yl)methylene)-3-phenylisoxazol-5(4H)-one;
(Z)-4-((5-(piperidin-1-yl)thiophen-2-yl)methylene)-3-(trifluoromethyl)isoxazol-5(4H)-one;
(Z)-3-phenyl-4-((5-(piperidin-1-yl)thiophen-2-yl)methylene)isoxazol-5(4H)-one;
(Z)-3-(perfluoroethyl)-4-((5-(piperidin-1-yl)thiophen-2-yl)methylene)isoxazol-5(4H)-one;
(Z)-3-(difluoromethyl)-4-((5-(piperidin-1-yl)thiophen-2-yl)methylene)isoxazol-5(4H)-one;
(Z)-4-((5-(pyrrolidin-1-yl)thiophen-2-yl)methylene)-3-(trifluoromethyl)isoxazol-5(4H)-one;
(Z)-3-phenyl-4-((5-(pyrrolidin-1-yl)thiophen-2-yl)methylene)isoxazol-5(4H)-one;
(Z)-4-((5-morpholinothiophen-2-yl)methylene)-3-(trifluoromethyl)isoxazol-5(4H)-one;
(Z)-4-((5-morpholinothiophen-2-yl)methylene)-3-phenylisoxazol-5(4H)-one;
tert-butyl (Z)-4-(5-((5-oxo-3-(trifluoromethyl)isoxazol-4(5H)-ylidene)methyl)thiophen-2-yl)piperazine-1-carboxylate;
tert-butyl (Z)-4-(5-((5-oxo-3-phenylisoxazol-4(5H)-ylidene)methyl)thiophen-2-yl)piperazine-1-carboxylate;
(Z)-3-phenyl-4-((5-(piperazin-1-yl)thiophen-2-yl)methylene)isoxazol-5(4H)-one;
(Z)-4-((5-(4-acetylpiperazin-1-yl)thiophen-2-yl)methylene)-3-phenylisoxazol-5(4H)-one;
(Z)-4-((5-(4-(methylsulfonyl)piperazin-1-yl)thiophen-2-yl)methylene)-3-phenylisoxazol-5(4H)-one;
(Z)-4-((5-(4-hydroxypiperidin-1-yl)thiophen-2-yl)methylene)-3-(trifluoromethyl)isoxazol-5(4H)-one;
(Z)-4-((5-(4-hydroxypiperidin-1-yl)thiophen-2-yl)methylene)-3-phenylisoxazol-5(4H)-one;
(Z)-4-((5-(4-oxopiperidin-1-yl)thiophen-2-yl)methylene)-3-(trifluoromethyl)isoxazol-5(4H)-one;
(Z)-4-((5-(4-oxopiperidin-1-yl)thiophen-2-yl)methylene)-3-phenylisoxazol-5(4H)-one;
(Z)-4-((5-thiomorpholinothiophen-2-yl)methylene)-3-(trifluoromethyl)isoxazol-5(4H)-one;
(Z)-3-phenyl-4-((5-thiomorpholinothiophen-2-yl)methylene)isoxazol-5(4H)-one;
(Z)-4-((5-(1,1-dioxidothiomorpholino)thiophen-2-yl)methylene)-3-phenylisoxazol-5(4H)-one;
(Z)-4-((5-(1-oxidothiomorpholino)thiophen-2-yl)methylene)-3-phenylisoxazol-5(4H)-one;
(Z)-4-((4-bromo-5-(dimethylamino)thiophen-2-yl)methylene)-3-(trifluoromethyl)isoxazol-5(4H)-one;
(Z)-4-((4-bromo-5-(dimethylamino)thiophen-2-yl)methylene)-3-phenylisoxazol-5(4H)-one;
(Z)-4-((5-(dimethylamino)-4-phenylthiophen-2-yl)methylene)-3-(trifluoromethyl)isoxazol-5(4H)-one;
(Z)-3-methyl-4-((5-(piperidin-1-yl)thiophen-2-yl)methylene)isoxazol-5(4H)-one;
(Z)-4-((5-(1,1-dioxidothiomorpholino)thiophen-2-yl)methylene)-3-(perfluoroethyl)isoxazol-5(4H)-one;
(Z)-4-((5-(1-oxidothiomorpholino)thiophen-2-yl)methylene)-3-(perfluoroethyl)isoxazol-5(4H)-one;
(Z)-4-((5-(bis(2-methoxyethyl)amino)thiophen-2-yl)methylene)-3-(trifluoromethyl)isoxazol-5(4H)-one;
(Z)-4-((5-(azepan-1-yl)thiophen-2-yl)methylene)-3-(trifluoromethyl)isoxazol-5(4H)-one;
(Z)-4-((5-(azetidin-1-yl)thiophen-2-yl)methylene)-3-(trifluoromethyl)isoxazol-5(4H)-one;
(Z)-4-((5-(3-hydroxypyrrolidin-1-yl)thiophen-2-yl)methylene)-3-(trifluoromethyl)isoxazol-5(4H)-one;
(Z)-1-(5-((5-oxo-3-(trifluoromethyl)isoxazol-4(5H)-ylidene)methyl)thiophen-2-yl)pyrrolidin-3-yl phenylcarbamate;
(Z)-4-((7-(dimethylamino)-2,3-dihydrothieno[3,4-b][1,4]dioxin-5-yl)methylene)-3-(trifluoromethyl)isoxazol-5(4H)-one;
(Z)-4-((5-(dimethylamino)thiophen-2-yl)methylene)-3-ethylisoxazol-5(4H)-one;
(Z)-3-(tert-butyl)-4-((5-(dimethylamino)thiophen-2-yl)methylene)isoxazol-5(4H)-one;
(Z)-3-(tert-butyl)-4-((5-(piperidin-1-yl)thiophen-2-yl)methylene)isoxazol-5(4H)-one;
(Z)-3-cyclopropyl-4-((5-(dimethylamino)thiophen-2-yl)methylene)isoxazol-5(4H)-one;
(Z)-4-((5-(dimethylamino)thiophen-2-yl)methylene)-3-(methoxymethyl)isoxazol-5(4H)-one;
(Z)-3-(butoxymethyl)-4-((5-(dimethylamino)thiophen-2-yl)methylene)isoxazol-5(4H)-one;
methyl (Z)-2-(4-((5-(dimethylamino)thiophen-2-yl)methylene)-5-oxo-4,5-dihydroisoxazol-3-yl)acetate;
butyl (Z)-2-(4-((5-(dimethylamino)thiophen-2-yl)methylene)-5-oxo-4,5-dihydroisoxazol-3-yl)acetate;
benzyl (Z)-2-(4-((5-(dimethylamino)thiophen-2-yl)methylene)-5-oxo-4,5-dihydroisoxazol-3-yl)acetate;
phenethyl (Z)-2-(4-((5-(dimethylamino)thiophen-2-yl)methylene)-5-oxo-4,5-dihydroisoxazol-3-yl)acetate;
ethyl (Z)-4-((5-(dimethylamino)thiophen-2-yl)methylene)-5-oxo-4,5-dihydroisoxazole-3-carboxylate;
butyl (Z)-4-((5-(dimethylamino)thiophen-2-yl)methylene)-5-oxo-4,5-dihydroisoxazole-3-carboxylate;
(Z)-4-(4-((5-(dimethylamino)thiophen-2-yl)methylene)-5-oxo-4,5-dihydroisoxazol-3-yl)benzonitrile;
(Z)-4-((5-(dimethylamino)thiophen-2-yl)methylene)-3-(4-(trifluoromethyl)phenyl)isoxazol-5(4H)-one;
(Z)-4-((5-(dimethylamino)thiophen-2-yl)methylene)-3-(4-methoxyphenyl)isoxazol-5(4H)-one;
(Z)-4-((5-(dimethylamino)thiophen-2-yl)methylene)-3-(pyridin-4-yl)isoxazol-5(4H)-one; or
(Z)-4-((4-chloro-5-(dimethylamino)thiophen-2-yl)methylene)-3-(trifluoromethyl)isoxazol-5(4H)-one.

11. The compound of claim 1 wherein the compound is (E)-4-((5-(dimethylamino)thiophen-2-yl)methylene)-3-(trifluoromethyl)isoxazol-5(4H)-one;
(E)-4-((5-(dimethylamino)thiophen-2-yl)methylene)-3-phenylisoxazol-5(4H)-one;
(E)-4-((5-(dimethylamino)thiophen-2-yl)methylene)-3-(perfluoroethyl)isoxazol-5(4H)-one;
(E)-3-(difluoromethyl)-4-((5-(dimethylamino)thiophen-2-yl)methylene)isoxazol-5(4H)-one;
(E)-4-((5-(dimethylamino)thiophen-2-yl)methylene)-3-methylisoxazol-5(4H)-one;
(E)-4-((5-(diethylamino)thiophen-2-yl)methylene)-3-(trifluoromethyl)isoxazol-5(4H)-one;
(E)-4-((5-(diethylamino)thiophen-2-yl)methylene)-3-phenylisoxazol-5(4H)-one;
(E)-4-((5-(dibutylamino)thiophen-2-yl)methylene)-3-phenylisoxazol-5(4H)-one;
(E)-4-((5-(dimethylamino)-3-methylthiophen-2-yl)methylene)-3-(trifluoromethyl)isoxazol-5(4H)-one;
(E)-4-((5-(dimethylamino)-3-methylthiophen-2-yl)methylene)-3-phenylisoxazol-5(4H)-one;
(E)-4-((5-(piperidin-1-yl)thiophen-2-yl)methylene)-3-(trifluoromethyl)isoxazol-5(4H)-one;
(E)-3-phenyl-4-((5-(piperidin-1-yl)thiophen-2-yl)methylene)isoxazol-5(4H)-one;
(E)-3-(perfluoroethyl)-4-((5-(piperidin-1-yl)thiophen-2-yl)methylene)isoxazol-5(4H)-one;
(E)-3-(difluoromethyl)-4-((5-(piperidin-1-yl)thiophen-2-yl)methylene)isoxazol-5(4H)-one;
(E)-4-((5-(pyrrolidin-1-yl)thiophen-2-yl)methylene)-3-(trifluoromethyl)isoxazol-5(4H)-one;
(E)-3-phenyl-4-((5-(pyrrolidin-1-yl)thiophen-2-yl)methylene)isoxazol-5(4H)-one;
(E)-4-((5-morpholinothiophen-2-yl)methylene)-3-(trifluoromethyl)isoxazol-5(4H)-one;
(E)-4-((5-morpholinothiophen-2-yl)methylene)-3-phenylisoxazol-5(4H)-one;
tert-butyl (E)-4-(5-((5-oxo-3-(trifluoromethyl)isoxazol-4(5H)-ylidene)methyl)thiophen-2-yl)piperazine-1-carboxylate;
tert-butyl (E)-4-(5-((5-oxo-3-phenylisoxazol-4(5H)-ylidene)methyl)thiophen-2-yl)piperazine-1-carboxylate;
(E)-3-phenyl-4-((5-(piperazin-1-yl)thiophen-2-yl)methylene)isoxazol-5(4H)-one;
(E)-4-((5-(4-acetylpiperazin-1-yl)thiophen-2-yl)methylene)-3-phenylisoxazol-5(4H)-one;
(E)-4-((5-(4-(methylsulfonyl)piperazin-1-yl)thiophen-2-yl)methylene)-3-phenylisoxazol-5(4H)-one;
(E)-4-((5-(4-hydroxypiperidin-1-yl)thiophen-2-yl)methylene)-3-(trifluoromethyl)isoxazol-5(4H)-one;
(E)-4-((5-(4-hydroxypiperidin-1-yl)thiophen-2-yl)methylene)-3-phenylisoxazol-5(4H)-one;
(E)-4-((5-(4-oxopiperidin-1-yl)thiophen-2-yl)methylene)-3-(trifluoromethyl)isoxazol-5(4H)-one;
(E)-4-((5-(4-oxopiperidin-1-yl)thiophen-2-yl)methylene)-3-phenylisoxazol-5(4H)-one;
(E)-4-((5-thiomorpholinothiophen-2-yl)methylene)-3-(trifluoromethyl)isoxazol-5(4H)-one;
(E)-3-phenyl-4-((5-thiomorpholinothiophen-2-yl)methylene)isoxazol-5(4H)-one;
(E)-4-((5-(1,1-dioxidothiomorpholino)thiophen-2-yl)methylene)-3-phenylisoxazol-5(4H)-one;
(E)-4-((5-(1-oxidothiomorpholino)thiophen-2-yl)methylene)-3-phenylisoxazol-5(4H)-one;
(E)-4-((4-bromo-5-(dimethylamino)thiophen-2-yl)methylene)-3-(trifluoromethyl)isoxazol-5(4H)-one;
(E)-4-((4-bromo-5-(dimethylamino)thiophen-2-yl)methylene)-3-phenylisoxazol-5(4H)-one;
(E)-4-((5-(dimethylamino)-4-phenylthiophen-2-yl)methylene)-3-(trifluoromethyl)isoxazol-5(4H)-one;
(E)-3-methyl-4-((5-(piperidin-1-yl)thiophen-2-yl)methylene)isoxazol-5(4H)-one;
(E)-4-((5-(1,1-dioxidothiomorpholino)thiophen-2-yl)methylene)-3-(perfluoroethyl)isoxazol-5(4H)-one;
(E)-4-((5-(1-oxidothiomorpholino)thiophen-2-yl)methylene)-3-(perfluoroethyl)isoxazol-5(4H)-one;
(E)-4-((5-(bis(2-methoxyethyl)amino)thiophen-2-yl)methylene)-3-(trifluoromethyl)isoxazol-5(4H)-one;
(E)-4-((5-(azepan-1-yl)thiophen-2-yl)methylene)-3-(trifluoromethyl)isoxazol-5(4H)-one;
(E)-4-((5-(azetidin-1-yl)thiophen-2-yl)methylene)-3-(trifluoromethyl)isoxazol-5(4H)-one;
(E)-4-((5-(3-hydroxypyrrolidin-1-yl)thiophen-2-yl)methylene)-3-(trifluoromethyl)isoxazol-5(4H)-one;
(E)-1-(5-((5-oxo-3-(trifluoromethyl)isoxazol-4(5H)-ylidene)methyl)thiophen-2-yl)pyrrolidin-3-yl phenylcarbamate;
(E)-4-((7-(dimethylamino)-2,3-dihydrothieno[3,4-b][1,4]dioxin-5-yl)methylene)-3-(trifluoromethyl)isoxazol-5(4H)-one;
(E)-4-((5-(dimethylamino)thiophen-2-yl)methylene)-3-ethylisoxazol-5(4H)-one;
(E)-3-(tert-butyl)-4-((5-(dimethylamino)thiophen-2-yl)methylene)isoxazol-5(4H)-one;
(E)-3-(tert-butyl)-4-((5-(piperidin-1-yl)thiophen-2-yl)methylene)isoxazol-5(4H)-one;
(E)-3-cyclopropyl-4-((5-(dimethylamino)thiophen-2-yl)methylene)isoxazol-5(4H)-one;
(E)-4-((5-(dimethylamino)thiophen-2-yl)methylene)-3-(methoxymethyl)isoxazol-5(4H)-one;
(E)-3-(butoxymethyl)-4-((5-(dimethylamino)thiophen-2-yl)methylene)isoxazol-5(4H)-one;
methyl (E)-2-(4-((5-(dimethylamino)thiophen-2-yl)methylene)-5-oxo-4,5-dihydroisoxazol-3-yl)acetate;
butyl (E)-2-(4-((5-(dimethylamino)thiophen-2-yl)methylene)-5-oxo-4,5-dihydroisoxazol-3-yl)acetate;
benzyl (E)-2-(4-((5-(dimethylamino)thiophen-2-yl)methylene)-5-oxo-4,5-dihydroisoxazol-3-yl)acetate;
phenethyl (E)-2-(4-((5-(dimethylamino)thiophen-2-yl)methylene)-5-oxo-4,5-dihydroisoxazol-3-yl)acetate;
ethyl (E)-4-((5-(dimethylamino)thiophen-2-yl)methylene)-5-oxo-4,5-dihydroisoxazole-3-carboxylate;
butyl (E)-4-((5-(dimethylamino)thiophen-2-yl)methylene)-5-oxo-4,5-dihydroisoxazole-3-carboxylate;
(E)-4-(4-((5-(dimethylamino)thiophen-2-yl)methylene)-5-oxo-4,5-dihydroisoxazol-3-yl)benzonitrile;
(E)-4-((5-(dimethylamino)thiophen-2-yl)methylene)-3-(4-(trifluoromethyl)phenyl)isoxazol-5(4H)-one;
(E)-4-((5-(dimethylamino)thiophen-2-yl)methylene)-3-(4-methoxyphenyl)isoxazol-5(4H)-one;
(E)-4-((5-(dimethylamino)thiophen-2-yl)methylene)-3-(pyridin-4-yl)isoxazol-5(4H)-one; or
(E)-4-((4-chloro-5-(dimethylamino)thiophen-2-yl)methylene)-3-(trifluoromethyl)isoxazol-5(4H)-one.

12. An optical filter comprising one or more compounds according to claim 1.

13. The optical filter according to claim 12, further comprising at least one chroma-enhancing compound that is not a compound of Formula I.

14. The optical filter according to claim 12, wherein the optical filter has a peak optical density of between about 0.05 and about 6.00 OD.

15. The optical filter according to claim 12, wherein the one or more compounds is present in the optical filter in a range of about 0.0002% weight to about 6% weight.

16. A lens for eyewear comprising a lens body and an optical filter, wherein the optical filter comprises one or more compounds according to claim 1.

17. The lens for eyewear according to claim 16, wherein the optical filter selectively blocks visible light within a spectral band of wavelengths ranging from about 475 nm to about 510 nm

18. The lens for eyewear according to claim 16, wherein the optical filter further comprises at least one chroma-enhancing compound that is not a compound of Formula I.

19. The lens for eyewear according to claim 16, wherein the optical filter has a peak optical density of between about 0.05 and about 6.00 OD.

20. The lens for eyewear according to claim 16, wherein the at least one or more compounds present in the lens in a range of about 0.0002% weight to about 6% weight.

21. Eyewear comprising a lens wherein the lens comprises a lens body; and an optical filter, wherein the optical filter comprises one or more compounds according to claim 1.

22. The eyewear according to claim 21, wherein the optical filter selectively blocks visible light within a spectral band of wavelengths ranging from about 475 nm to about 510 nm

23. The eyewear according to claim 21, wherein the optical filter further comprises at least one chroma-enhancing compound that is not a compound of Formula I.
